(19) United States
(12) Patent Application Publication
(10) Pub. No.: US 2015/0330984 A1
(43) Pub. Date: Nov. 19, 2015

(54) METABOLOMIC PROFILING DEFINES ONCOGENES DRIVING PROSTATE TUMORS

(71) Applicant: Dana-Farber Cancer Institute, Inc., Boston, MA (US)

(72) Inventors: Massimo Loda, Belmont, MA (US); Carmen Priolo, Brookline, MA (US); Saumyadipta Pyne, Hyderabad (IN)

(73) Assignee: Dana-Farber Cancer Institute, Inc., Boston, MA (US)

(21) Appl. No.: 14/649,045

(22) PCT Filed: Dec. 6, 2013
(86) PCT No.: PCT/US13/73569

§ 371 Date: Jun. 2, 2015

Related U.S. Application Data
(60) Provisional application No. 61/779,446, filed on Mar. 13, 2013.
(60) Provisional application No. 61/734,040, filed on Dec. 6, 2012.

(51) Int. Cl. G01N 33/574; C12Q 1/68

(52) U.S. Cl. CPC G01N 33/57434; C12Q 1/6886; C12Q 2600/156

(57) ABSTRACT

The invention provides methods and products to identify metabolic status of Akt1 and Myc in tumors, and to treat cancer. The method comprises performing an assay to measure a profile of metabolites in a prostate tumor sample obtained from a subject, wherein the metabolites are differentially produced in prostate tumors with high Akt1 expression versus prostate tumors with high Myc expression; and comparing, with at least one processor, the profile of metabolites with an appropriate reference profile of the metabolites to assign an Akt1 and Myc metabolic status to the sample based on results of the comparison.

### RELATED APPLICATIONS

This application claims the benefit under 35 U.S.C. §119(e) of U.S. Provisional Application Nos. 61/734,040, filed Dec. 6, 2012, and 61/779,446, filed Mar. 13, 2013, the entire contents of which are hereby incorporated by reference.

### FEDERALLY SPONSORED RESEARCH

This invention was made with Government support under National Institute of Health (NIH) Grant R01 CA131945. Accordingly, the Government has certain rights in this invention.

### BACKGROUND OF THE INVENTION

Prostate cancer is the most common cause of death from cancer in men over age 75. Many factors, including genetics and diet, have been implicated in the development of prostate cancer. Proliferation in normal cells occurs when nutrients are taken up from the environment as a result of stimulation by growth factors. Cancer cells overcome this growth factor dependence either by acquiring genetic mutations that result in altered metabolic pathways or by affecting metabolic pathways de novo with targeted mutations in critical metabolic enzymes. Altered metabolic pathways, in turn, stimulate cell growth by either providing fuel for energy or by efficiently incorporating nutrients into biomass.

Metabolic alterations may occur as a result of altered pathways, in turn a consequence of genetic events. Alternatively, metabolic alterations may be primary events in cancer but require genetic alterations in critical pathways for oncogenesis. A fundamental unanswered question is whether all oncogenic drivers (such as Myc or Akt) harness a similar metabolic response or whether each oncogenic event results in its own specific metabolic program. This is important because if the latter is true, targeting selected metabolic enzymes/pathways together with the putative driving oncogenes could become a powerful and targeted approach in cancer therapeutics.

### SUMMARY OF THE INVENTION

It has been discovered, surprisingly, that metabolic profiles are specific to oncogenes driving human tumors, specifically prostate tumor. Accordingly, in some aspects, the invention involves identifying Akt1 and Myc status in a prostate tumor by performing an assay to measure a profile of metabolites in a prostate tumor sample obtained from a subject, wherein the metabolites are differentially produced in prostate tumors with high Akt1 expression versus prostate tumors with high Myc expression, and comparing, with at least one processor, the profile of metabolites with an appropriate reference profile of the metabolites to assign an Akt1 and Myc status to the sample based on results of the comparison.

According to some aspects of the invention, a method to identify Akt1 and Myc status in a prostate tumor is provided. The method comprises analyzing, with at least one processor, a profile of a set of metabolites in a prostate tumor sample obtained from a subject to assign an Akt1 and Myc status to the sample, wherein the metabolites are differentially produced in prostate tumors with high Akt1 expression versus prostate tumors with high Myc expression, and the profile of metabolites is compared to an appropriate reference profile of the metabolites.

In some embodiments, the appropriate reference profile of the metabolites comprises profiles of the metabolites in prostate tumor with high Akt1 expression, in prostate tumor with low Akt1 expression, in prostate tumor with high Myc expression, and in prostate tumor with low Myc expression. In some embodiments, the metabolic profile comprises at least 5, at least 10, at least 25, at least 50, at least 75, at least 100, at least 125, at least 150, at least 175, at least 200, at least 225, at least 250, at least 275, at least 300, at least 350, at least 375, at least 400 metabolites, at least 450 metabolites, at least 500 metabolites, at least 1000 metabolites, or at least 1500 metabolites. In some embodiments, the metabolic profile of the tumor sample is measured using one or more of mass spectroscopy, nuclear magnetic resonance or chromatography. In some embodiments, the metabolites are selected from Table 1. In some embodiments, the computer assigns a status of high Akt1/high Myc, high Akt1/low Myc, low Akt1/high Myc, or low Akt1/low Myc to the sample. In some embodiments, the profile of metabolites of the tumor sample is compared using cluster analysis. In some embodiments, the cluster analysis is selected from the group consisting of: hierarchical clustering, k-mean clustering, distribution-based clustering, and density-based clustering. In some embodiments, the differentially produced metabolites are selected using a threshold of p value <0.05. In some embodiments, the methods described herein further comprise determining a confidence value for the Akt1 and Myc status assigned to the sample and providing an indication of the confidence value and the Akt1 and Myc status assigned to the sample to a user.

According to some aspects of the invention, a method to treat prostate tumor is provided. The method comprises obtaining a prostate tumor sample from a subject, measuring a metabolic profile of the tumor sample, wherein the metabolites are differentially produced in prostate tumors with high Akt1 expression versus prostate tumors with high Myc expression, comparing the metabolic profile to an appropriate reference profile of the metabolites, and treating the subject with an Akt1 inhibitor when results of the comparison of the metabolic profile indicate high Akt1 expression in the tumor sample and/or treating the subject with a Myc inhibitor when results of the comparison of the metabolic profile indicate high Myc in the tumor sample.

In some embodiments, the Akt1 inhibitor is selected from the group consisting of (a) a low molecular weight compound or high molecular weight compound which inhibits the phosphorylation of Akt1, (b) a low molecular weight compound or high molecular weight compound which inhibits the expression of Akt1, (c) an antibody which inhibits the phosphorylation of Akt1, (d) an antibody which inhibits the expression of Akt1, (e) a siRNA or shRNA against a polynucleotide encoding Akt1, (f) an antisense polynucleotide comprising a nucleotide sequence complementary or substantially complementary to the nucleotide sequence of a polynucleotide encoding Akt1, or comprising a part of said nucleotide sequence, (g) a ribozyme directed to a polynucleotide encoding Akt1, (h) a mutant of Akt1 which dominant-negatively acts on Akt1 or a polynucleotide encoding said mutant, and (i) an aptamer against Akt1. In some embodiments, the Akt1 inhibitor is Perifosine, Miltefosine MK02206, GSK690693, GDC-0068, or AZD5363.

In some embodiments, the Myc inhibitor is selected from the group consisting of (a) a low molecular weight compound or high molecular weight compound which inhibits the expression of Myc, (b) an antibody which inhibits the expression of Myc, (e) a siRNA or shRNA against a polynucleotide encoding Myc, (f) an antisense polynucleotide comprising a nucleotide sequence complementary or substantially complementary to the nucleotide sequence of a polynucleotide encoding Myc, or comprising a part of said nucleotide sequence, (g) a ribozyme directed to a polynucleotide encoding Myc, (h) a mutant of Myc which dominant-negatively acts on Myc or a polynucleotide encoding said mutant, and (i) an aptamer against Myc. In some embodiments, the Myc inhibitor is selected from the group consisting of 10058-F4, JQ1 and Omomyc.

In some embodiments, the metabolic profile of the tumor sample is measured using one or more of mass spectroscopy, nuclear magnetic resonance, or chromatography. In some embodiments, the metabolites are selected from Table 1. In some embodiments, the metabolic profile of the tumor sample is compared using cluster analysis. In some embodiments, the cluster analysis is selected from the group consisting of: hierarchical clustering, k-mean clustering, distribution-based clustering, and density-based clustering. In some embodiments, the appropriate reference profile of the metabolites comprises profiles of the metabolites in prostate tumor with high Akt1 expression, in prostate tumor with low Akt1 expression, in prostate tumor with high Myc expression, and in prostate tumor with low Myc expression. In some embodiments, the metabolic profile comprises at least 5, at least 10, at least 25, at least 50, at least 75, at least 100, at least 125, at least 150, at least 175, at least 200, at least 225, at least 250, at least 275, at least 300, at least 350, at least 375, at least 400 metabolites, at least 450 metabolites, at least 500 metabolites, at least 1000 metabolites, or at least 1500 metabolites. In some embodiments, the differentially produced metabolites are selected using a threshold of p value <0.05.

According to some aspects of the invention, a method to treat prostate tumor is provided. The method comprises obtaining a biological sample from a subject, measuring a level of sarcosine in the sample, comparing the level of sarcosine in the sample to a control sarcosine level, and treating the subject with a Myc inhibitor when the measured level of sarcosine in the sample is increased relative to the control level.

In some embodiments, the Myc inhibitor is selected from the group consisting of (a) a low molecular weight compound or high molecular weight compound which inhibits the expression of Myc, (b) an antibody which inhibits the expression of Myc, (e) a siRNA or shRNA against a polynucleotide encoding Myc, (f) an antisense polynucleotide comprising a nucleotide sequence complementary or substantially complementary to the nucleotide sequence of a polynucleotide encoding Myc, or comprising a part of said nucleotide sequence, (g) a ribozyme directed to a polynucleotide encoding Myc, (h) a mutant of Myc which dominant-negatively acts on Myc or a polynucleotide encoding said mutant, and (i) an aptamer against Myc. In some embodiments, the Myc inhibitor is selected from the group consisting of 10058-F4, JQ1 and Omomyc. In some embodiments, the level of sarcosine in the sample is measured using one or more of mass spectroscopy, nuclear magnetic resonance or chromatography. In some embodiments, the biological sample is selected from the group consisting of a urine, blood, serum, plasma, and tissue sample.

According to some aspects of the invention, a method to identify Akt1 and Myc status in a prostate tumor is provided. The method comprises performing an assay to measure a profile of metabolites in a prostate tumor sample obtained from a subject, and comparing, with at least one processor, the profile of metabolites with a reference profile of the metabolites, the reference profile of the metabolites being profiles of the metabolites from prostate tumors with high Akt1 expression and from prostate tumors with high Myc expression, to assign an Akt1 and Myc status to the sample based on results of the comparison.

According to some aspects of the invention, a method to identify Akt1 and Myc status in a prostate tumor is provided. The method comprises performing an assay to measure a profile of metabolites in a prostate tumor sample obtained from a subject, and comparing the profile of metabolites with reference profiles of the metabolites with at least one processor programmed to recognize profiles of high Akt1 versus low Akt1 expressing tumors and high Myc versus low Myc expressing tumors, and assigning, with at least one processor, an Akt1 and Myc status to the sample based on results of the comparison.

In some embodiments, the methods described herein further comprise determining a confidence value for the Akt1 and Myc status assigned to the sample, and providing an indication of the confidence value and the Akt1 and Myc status assigned to the sample to a user. In some embodiments, the methods described herein further comprise determining whether the confidence value is below a threshold value, and providing an indication that the confidence value is below the threshold value.

According to some aspects of the invention, a computer-readable storage medium is provided. The storage medium is encoded with a plurality of instructions that, when executed by at least one processor, performs a method comprising comparing the profile of metabolites with reference profiles of the metabolites with at least one processor programmed to recognize profiles of high Akt1 versus low Akt1 expressing tumors and high Myc versus low Myc expressing tumors, and assigning, with at least one processor, an Akt1 and Myc status to the sample based on results of the comparison.

In some embodiments, the method further comprises determining a confidence value for the Akt1 and Myc status assigned to the sample, and providing an indication of the confidence value and the Akt1 and Myc status assigned to the sample to a user.

In some embodiments, the method further comprises determining whether the confidence value is below a threshold value, and providing an indication that the confidence value is below the threshold value.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1. Classification of prostate tumors by genomics and protein expression levels. The Venn diagram in (A) shows the number of tumors characterized by both copy number change at the PTEN or MYC locus and high phosphoAKT1 or MYC expression levels, and the number of those with either one alteration. Twelve and eleven tumors harbor 10q23.31 (PTEN locus) loss and 8q24.3 (MYC locus) gain, respectively, representing only 26% (7/27) of phosphoAKT1-high and 13% (2/15) of MYC-high tumors. K-means clustering was used to segregate 4 prostate tumor subgroups, i.e. phosphoAKT1-high/MYC-high (black dots), phosphoAKT1-high/MYC-low (red dots), phosphoAKT1-low/MYC-high (green dots) and phosphoAKT1-low/MYC-low (grey dots) (B).

FIG. 2. Enrichment of metabolic pathways across classes and systems. In heatmaps (A) through (C) the normalized enrichment scores of the most significantly enriched pathways within each of the 3 systems—cells, mice and human tumors are shown. Each row represents a KEGG pathway and each column an individual sample. Brown/green colors are used to denote high/low enrichment. Hierarchical clustering is used for unsupervised identification of the higher-level enrichment classes, which are well preserved across all 3 systems. The phenotypic labels of the samples are indicated as by a colored band on top of the heatmap, while the dendrogram represents the distances among them. In plot (D), we summarize the overall differential enrichments across the two classes of samples, Akt versus Myc, with simultaneous metabolic set enrichment analysis (akin to gene set enrichment analysis) measurements in all 3 systems. This information is depicted as points in 3-dimensional space, where each point represents a particular pathway, and each dimension a system. Enrichment of a pathway in Akt versus Myc overexpressed classes are given by positive and negative scores respectively. The top 5 positively enriched pathways (i.e. in high Akt samples) in all 3 systems, and the top 2 negatively enriched pathways (i.e. in high Myc samples) in all 3 systems, as chosen with an enrichment p-value threshold of 0.05, are highlighted as red and green points respectively.

FIG. 3. Relative mRNA expression of metabolic genes in RWPE-1 engineered cells. (A) Glucose metabolism; (B) Lipid metabolism; (C) Glutamine metabolism. (D) Diagram showing metabolic enzymes up-regulated in RWPE-AKT (red), RWPE-MYC (green) cells relative to control (blue) or to each other. (E) For each pathway, its normalized enrichment scores in each system and their average are shown. The top 5 most enriched pathways in the high-Akt samples across all 3 systems are shown in red. The top 5 most enriched pathways in the high-Myc samples across all 3 systems are shown in green. Also shown in light green that some pathways which have high enrichments in Akt-high both mice and human tumors have low enrichments in cells. (F) Relative mRNA levels of GLUT-1 in human prostate tumors.

FIG. 4 is an illustrative implementation of a computer system.

### DETAILED DESCRIPTION OF THE INVENTION

A fundamental unanswered question in cancer biology has been whether metabolic changes are similar in cancers driven by different oncogenes or whether each genetic alteration induces a specific metabolic profile. This invention is based, at least in part, on the surprising discovery that metabolic profiles are specific to oncogenes driving human tumors, specifically prostate cancer. Thus, prostate tumors exhibit metabolic fingerprints of their molecular phenotypes, which impacts metabolic diagnostics and targeted therapeutics. Accordingly, aspects of the invention relate to methods aim at indirectly identifying Akt1 and Myc-driven tumors, and methods to treat cancer. The metabolic profiles of the tumors are compared to appropriate reference metabolic profiles to determine if the tumor is “driven” by either Akt1 or Myc oncogenes. This methodology can also be applied to other oncogenes (or tumor suppressor genes), combination of these and to any other type of cancer.

According to some aspects of the invention, a method to identify Akt1 and Myc status in a prostate tumor is provided. The method comprises performing an assay to measure a profile of metabolites in a prostate tumor sample obtained from a subject, wherein the metabolites are differentially produced in prostate tumors with high Akt1 expression versus prostate tumors with high Myc expression; and comparing, with at least one processor, the profile of metabolites with an appropriate reference profile of the metabolites to assign an Akt1 and Myc status to the sample based on results of the comparison.

The AKT1 (v-akt murine thymoma viral oncogene homolog 1, also called AKT) gene encodes a serine/threonine-protein kinase that is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt1 was originally identified as the oncogene in the transforming retrovirus, AKT8 (Staal S P et al. (July 1977) “Isolation of transforming murine leukemia viruses from mice with a high incidence of spontaneous lymphoma”. Proc. Natl. Acad. Sci. U.S.A. 74 (7): 3065-7).

Akt possesses a protein domain known as Pleckstrin Homology (PH) domain, which binds either PIP3 (phosphatidylinositol (3,4,5)-trisphosphate, PtdIns(3,4,5)P3) or PIP2 (phosphatidylinositol (3,4)-bisphosphate, PtdIns(3,4)P2). PI 3-kinases (phosphoinositide 3-kinase or PI3-K) are activated on receipt of chemical messengers which tell the cell to begin the growth process. For example, PI 3-kinases may be activated by a G protein coupled receptor or receptor tyrosine kinase such as the insulin receptor. Once activated, PI 3-kinase phosphorylates PIP2 to form PIP3. PI3K-generated PIP3 and PIP2 recruit Akt1 to the plasma membrane where it becomes phosphorylated by its activating kinases, such as, phosphoinositide dependent kinase 1 (PDK1). This phosphorylation leads to activation of Akt1.

As used herein “Myc” refers to a family of genes and corresponding polypeptides. The Myc family encompasses Myc proteins having Myc transcriptional activity, including but not limited to, c-Myc (GenBank Accession No P01106), N-Myc (GenBank Accession No P04198), L-Myc (GenBank Accession No. CAA30249), S-Myc (GenBank Accession No. BAA37155) and B-Myc (GenBank Accession No. NP_{—}075815).

Myc is a regulator gene that encodes a transcription factor. Myc proteins are most closely homologous at the MB1 and MB2 regions in the N-terminal region and at the basic helix-loop-helix leucine zipper (bHLHLZ) motif in the C-terminal region (Osier et al. (2002) Adv Cancer Res 84:81-154; Grandori et al. (2000) Annu Rev Cell Dev Biol 16:653-699). In the human genome, Myc is located on chromosome 8 and is believed to regulate expression of 15% of all genes through binding Enhancer Box sequences (E-boxes) and recruiting histone acetyltransferases (HATs). By modifying the expression of its target genes, Myc activation results in numerous biological effects. The first to be discovered was its capability to drive cell proliferation (upregulates cyclins, downregulates p21), but it also plays a very important role in regulating cell growth (upregulates ribosomal RNA and proteins), apoptosis (downregulates Bcl-2), differentiation and stem cell self-renewal. Myc is a very strong proto-oncogene and it is very often found to be upregulated in many types of cancers.

Between 30 and 70% of prostate tumors have genomic loss of phosphatase and tensin homolog (PTEN), leading to constitutively active phosphatidylinositol 3-kinase/protein Kinase B (PI3K/AKT) pathway, while 8q amplification including the MYC gene occurs in ˜30% of prostate tumors. Thus, these are recognized as the most frequent genetic alterations in prostate tumors. Both activated Akt and especially Myc overexpression faithfully reproduce the stages of human prostate carcinogenesis in genetically engineered mice (GEMMs). Recent literature shows that MYC promotes glutaminolysis, whereas AKT activation is associated with enhanced aerobic glycolysis and/or increased expression of glycolytic enzymes in different cell types, including prostate. However, the impact of these oncogenes or the genomic alterations causing their activation on the metabolome of human prostate tumors had not been fully elucidated.

“Assign an Akt1 status” means identifying, with at least one processor, the sample as having a metabolite profile that is similar to or characteristic of a prostate tumor with high Akt1 expression or with low Akt1 expression. “Assign a Myc status” means identifying, with at least one processor, the sample as having a metabolite profile that is similar to or characteristic of a prostate tumor with high Myc expression or with low Myc expression. In some embodiments, the sample is assigned by the processor a metabolic status of high Akt1/high Myc, high Akt1/low Myc, low Akt1/high Myc, or low Akt1/low Myc.

As used herein, a “high Akt1” or a “high Myc” metabolic status indicates that the expression level of Akt1 or Myc in the sample is similar to or characteristic of prostate tumors having constitutively activated (phosphorylated) Ak1 or prostate tumors overexpressing Myc. In some embodiments, a “high Akt1” or a “high Myc” status indicates that the expression level of Akt1 or Myc in the sample is similar to or characteristic of prostate cells having constitutively activated (phosphorylated) Akt1 or overexpressing Myc. In some embodiments, a “high Akt1” status indicates that the expression level of Akt1 in the sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher than that in prostate tumors or prostate cells in which Akt1 is not constitutively activated. In some embodiments, a “high Myc” status indicates that the expression level of Myc in the sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher than that in prostate tumors or prostate cells in which Myc is not overexpressed.

Conversely, a “low Akt1” status indicates that the expression level of Akt1 in the sample is similar to or characteristic of prostate tumors or prostate cells in which Akt1 is not constitutively activated. A “low Myc” status indicates that the expression level of Myc in the sample is similar to or characteristic of prostate tumors or prostate cells in which Myc is not overexpressed. In some embodiments, a “low Akt1” or a “low Myc” status indicates that the expression level of Akt1 or Myc in the sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more lower than that in prostate tumors or prostate cells in which Akt1 is not constitutively activated or Myc is not overexpressed.

As used herein, “metabolites” are small molecule compounds, such as substrates for enzymes of metabolic pathways, intermediates of such pathways or the products produced by a metabolic pathway. Metabolic pathways are well known in the art, and include, for example, citric acid cycle, respiratory chain, glycolysis, gluconeogenesis, hexose monophosphate pathway, oxidative pentose phosphate pathway, production and β-oxidation of fatty acids, urea cycle, amino acid biosynthesis pathways, protein degradation pathways, amino acid degrading pathways, and biosynthesis or degradation of lipids, proteins, and nucleic acids. Accordingly, small molecule compound metabolites may be composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds.

Preferably, a metabolite has a molecular weight of 50 Da (Dalton) to 30,000 Da, most preferably less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. Preferably, a metabolite has, however, a molecular weight of at least 50 Da. Most preferably, a metabolite in accordance with the present invention has a molecular weight of 50 Da up to 1,500 Da.

In some embodiments, at least some of the metabolites used in the methods described herein are differentially produced in prostate tumors with high Akt1 expression versus prostate tumors with high Myc expression. In some embodiments, the metabolites that are differentially produced in prostate tumors with high Akt1 expression versus prostate tumors with high Myc expression are used in the methods described herein. By “differentially produced” it means that the average level of a metabolite in subjects with prostate tumors having high Akt1 expression has a statistically significant difference from that in subjects with prostate tumors having high Myc expression. For example, a significant difference that indicates differentially produced metabolite may be detected when the metabolite is present in prostate tumor with high Akt1 expression and absent in a prostate tumor with high Myc expression or vice versa. A significant difference that indicates differentially produced metabolite may be detected when the level of the metabolite in a prostate tumor sample of a subject with high Akt1 expression is at least 1%, at least 5%, at least 10%, at least 25%, at least 50%, at least 100%, at least 250%, at least 500%, or at least 1000% higher, or lower, than that of a subject with high Myc expression. Similarly, a significant difference may be detected when the level of a metabolite in a prostate tumor sample of a subject with high Akt1 expression is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than that of a subject with high Myc expression. Significant differences may be identified by using an appropriate statistical test. Tests for statistical significance are well known in the art and are exemplified in Applied Statistics for Engineers and Scientists by Petruccelli, Chen and Nandram 1999 Reprint Ed. In some embodiments, the differentially produced metabolites are selected using a criteria of false discovery rate <0.2. In some embodiments, the differentially produced metabolites are selected using a criteria of p value <0.05. In some embodiments, the metabolites used in the methods described herein are selected from Table 1 or Table 2. In some embodiments, the metabolites used in the methods described herein comprise at least 5, at least 10, at least 20, at least 30, at least 40, at least 50, at least 75, at least 100, at least 200, at least 300 of the metabolites described in Table 1 or Table 2.

As used herein, a “subject” refers to mammal, including humans and non-humans, such as primates. Typically the subject is a male human, and has been diagnosed as having a prostate tumor. In some embodiments, the subject may be diagnosed as having prostate tumor using one or more of the following tests: digital rectal exam (DRE), prostate imaging, biopsy with Gleason grading evaluation, presence of tumor markers such as prostate-specific antigen (PSA) and prostate cancer staging (Lumen et al. Screening and early diagnosis of prostate cancer: an update. Acta Clin Belg. 2012 July-August; 67(4):270-5). In some embodiments, the subject has one or more clinical symptoms of prostate tumor. A variety of clinical symptoms of prostate cancer are known in the art. Examples of such symptoms include, but are not limited to, frequent urination, nocturia (increased urination at night), difficulty starting and maintaining a steady stream of urine, hematuria (blood in the urine), dysuria (painful urination) and bone pain.

Cancer or neoplasia is characterized by deregulated cell growth and division. A tumor arising in a tissue originating from endoderm or exoderm is called a carcinoma, and one arising in tissue originating from mesoderm is known as a sarcoma (Darnell, J. (1990) Molecular Cell Biology, Third Ed., W.H. Freeman, NY). Cancers may originate due to a mutation in an oncogene, or by inactivation of a tumor-suppressing genes (Weinberg, R. A. (September 1988) Scientific Amer. 44-51). Examples of cancers include, but are not limited to cancers of the nervous system, breast, retina, lung, skin, kidney, liver, pancreas, genito-urinary tract, gastrointestinal tract, cancers of bone, and cancers of hematopoietic origin such as leukemias and lymphomas. In one embodiment of the present invention, the cancer is prostate cancer.

In some embodiments, the methods described herein are performed using a biological sample obtained from a subject. The term “biological sample” refers to a sample derived from a subject, e.g., a patient. Non-limiting examples of the biological sample include blood, serum, urine, and tissue. In some embodiments, the biological sample is a prostate tumor sample. Obtaining a prostate tumor sample from a subject means taking possession of a prostate tumor sample of the subject. In some embodiments, the person obtaining a prostate tumor sample from a subject and performing an assay to measure a profile of metabolites in the sample does not necessarily obtain the sample from the subject. In some embodiments, the sample may be removed from the subject by a medical practitioner (e.g., a doctor, nurse, or a clinical laboratory practitioner), and then provided to the person performing the assay to measure a profile of metabolites. The sample may be provided to the person performing an assay to measure the profile of metabolites by the subject or by a medical practitioner (e.g., a doctor, nurse, or a clinical laboratory practitioner). In some embodiments, the person performing an assay to measure the profile of metabolites obtains a prostate tumor sample from the subject by removing the sample from the subject.

It is to be understood that a prostate tumor sample may be processed in any appropriate manner to facilitate measuring profiles of metabolites. For example, biochemical, mechanical and/or thermal processing methods may be appropriately used to isolate a biomolecule of interest from a prostate tumor sample. The levels of the metabolites may also be determined in a prostate tumor sample directly. The levels of the metabolites may be measured by performing an assay, such as but not limited to, mass spectroscopy, positron emission tomography, gas chromatography (GC-MS) or HPLC liquid chromatography (LC-MS), [(18)F]-fluorodeoxyglucose positron emission tomography (FDG-PET), and magnetic resonance spectroscopic imaging (MRSI). Other appropriate methods for determining levels of metabolites will be apparent to the skilled artisan.

The methods disclosed herein typically comprise performing an assay to measure a profile of metabolites and comparing, with at least one processor, the profile of the metabolites to an appropriate reference profile. In some embodiments, the levels of at least 5, at least 10, at least 25, at least 50, at least 75, at least 100, at least 125, at least 150, at least 175, at least 200, at least 225, at least 250, at least 500, at least 750, at least 1000 or at least 1500 metabolites are measured and compared to assign an Akt1 and Myc status to the sample based on results of the comparison.

The assigned Akt1 and Myc status along with additional information such as the results of a PSA test and prostate imaging, can be used to determine the therapeutic options available to the subject. A report summarizing the results of the analysis, i.e. the assigned Akt1 and Myc status of the sample and any other information pertaining to the analysis could optionally be generated as part of the analysis (which may be interchangeably referred to herein as “providing” a report, “producing” a report, or “generating” a report). Examples of reports may include, but are not limited to, reports in paper (such as computer-generated printouts of test results) or equivalent formats and reports stored on computer readable medium (such as a CD, computer hard drive, or computer network server, etc.). Reports, particularly those stored on computer readable medium, can be part of a database (such as a database of patient records, which may be a “secure database” that has security features that limit access to the report, such as to allow only the patient and the patient's medical practitioners to view the report, for example). In addition to, or as an alternative to, generating a tangible report, reports can also be displayed on a computer screen (or the display of another electronic device or instrument).

A report can further be transmitted, communicated or reported (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party intended to view or possess the report. The act of ‘transmitting’ or ‘communicating’ a report can be by any means known in the art, based on the form of the report, and includes both oral and non-oral transmission. Furthermore, “transmitting” or “communicating” a report can include delivering a report (“pushing”) and/or retrieving (“pulling”) a report. For example, non-oral reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

The Akt1 and Myc status of the sample isolated from a subject is assigned by comparing the profile of metabolites of the sample to an appropriate reference profile of the metabolites. An appropriate reference profile of the metabolites can be determined or can be a pre-existing reference profile. An appropriate reference profile includes profiles of the metabolites in prostate tumor with high Akt1 expression (i.e. prostate tumor or prostate cells having constitutively activated (phosphorylated) Ak1), in prostate tumor with low Akt1 expression (i.e. prostate tumor or prostate cells not having constitutively activated Ak1), in prostate tumor with high Myc expression (i.e. prostate tumor or prostate cells overexpressing Myc), and in prostate tumor with low Myc expression (i.e. prostate tumor or prostate cells not overexpressing Myc). A lack of a significant difference between the metabolic profile determined from the subject and the appropriate reference profile is indicative of the Akt1 and Myc status of the sample.

In some embodiments, the methods described herein involve using at least one processor programmed to recognize profiles of high Akt1 versus low Akt1 expressing tumors and high Myc versus low Myc expressing tumors to assign an Akt1 and Myc status to the sample. The at least one processor assigns an Akt1 and Myc status to the sample isolated from the subject based on the profile of the metabolites of the sample. Typically the at least one processor is programmed using samples for which the Akt1 and Myc status has already been ascertained. Once the at least one processor is programmed, it may be applied to metabolic profiles obtained from a prostate tumor sample in order to assign an Akt1 and Myc status to the sample isolated from the subject. Thus, the methods may involve analyzing the metabolic profiles using one or more programmed processors to assign an Akt1 and Myc status to the sample based on the levels of the metabolites. The subject may be further diagnosed, e.g., by a health care provider, based on the assigned status.

The at least one processor may be programmed to assign a Akt1 and Myc status to a sample using one or more of a variety of techniques known in the art. For example, the at least one processor may be programmed to assign a Akt1 and Myc status using techniques including, but not limited to, logistic regression, partial least squares, linear discriminant analysis, regularized regression, quadratic discriminant analysis, neural network, naïve Bayes, C4.5 decision tree, k-nearest neighbor, random forest, and support vector machine. The at least one processor may be programmed to assign a Akt1 and Myc status to a sample using a data set comprising profiles of the metabolites that are produced in high Akt1 prostate tumors, low Akt1 prostate tumors, high Myc prostate tumors and low Myc prostate tumors. The data set may also comprise metabolic profiles of control individuals identified as not having prostate tumor.

In some embodiments, the at least one processor is programmed to assign a Akt1 and Myc status to a sample using cluster analysis. Cluster analysis or clustering refers to assigning a objects in a set of objects into groups (called clusters) so that the objects in the same cluster are more similar (in some sense or another) to each other than to those in other clusters. Cluster analysis itself is not embodied in a single algorithm, but describes a general task to be solved. Cluster analysis may be performed using various algorithms that differ significantly in their notion of what constitutes a cluster and how to efficiently find them. Popular notions of clusters include groups with small distances among the cluster members, dense areas of the data space, intervals or particular statistical distributions. In some embodiments, one or more particular algorithms used to perform cluster analysis are selected from the group consisting of: hierarchical clustering, k-mean clustering, distribution-based clustering, and density-based clustering.

A confidence value can also be determined to specify the degree of confidence with which the at least one programmed processor has classified a biological sample. There may be instances in which a sample is tested, but does not belong, or cannot be reliably assigned a particular classification with sufficient confidence. This evaluation may be performed by utilizing a threshold in which a sample having a confidence value below the determined threshold is a sample that cannot be classified with sufficient confidence (e.g., a “no call”). In such instances, the classifier may provide an indication that the confidence value is below the threshold value. In some embodiments, the sample is then manually classified to assign an Akt1 and Myc status to the sample.

As will be appreciated by the skilled artisan, the strength of the status assigned to a sample by the at least one programmed processor may be assessed by a variety of parameters including, but not limited to, the accuracy, sensitivity, specificity and area under the receiver operation characteristic curve. Methods for computing accuracy, sensitivity and specificity are known in the art. The at least one programmed processor may have an accuracy of at least 60%, at least 65%, at least 70%, at least 75%, at least 80%, at least 85%, at least 90%, at least 95%, at least 99%, or more. The at least one programmed processor may have an accuracy score in a range of about 60% to 70%, 70% to 80%, 80% to 90%, or 90% to 100%. The at least one programmed processor may have a sensitivity score of at least 60%, at least 65%, at least 70%, at least 75%, at least 80%, at least 85%, at least 90%, at least 95%, at least 99%, or more. The at least one programmed processor may have a sensitivity score in a range of about 60% to 70%, 70% to 80%, 80% to 90%, or 90% to 100%. The at least one programmed processor may have a specificity score of at least 60%, at least 65%, at least 70%, at least 75%, at least 80%, at least 85%, at least 90%, at least 95%, at least 99%, or more. The at least one programmed processor may have a specificity score in a range of about 60% to 70%, 70% to 80%, 80% to 90%, or 90% to 100%.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one non-transitory computer-readable storage medium (e.g., a computer memory, a USB drive, a flash memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

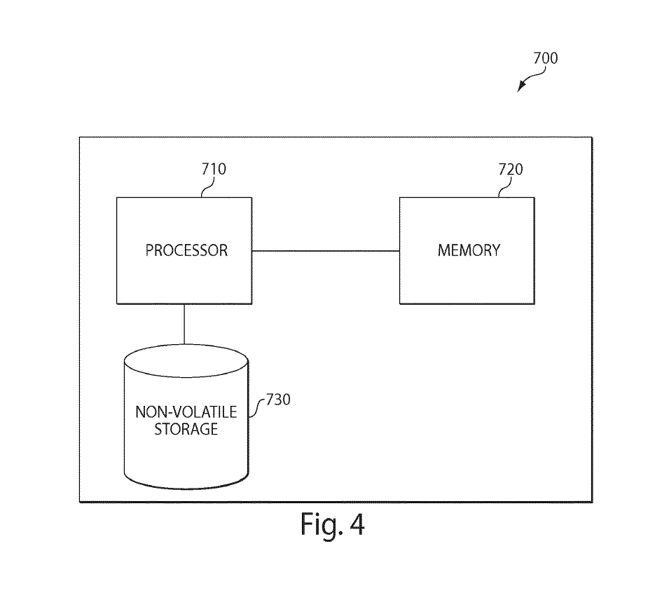

An illustrative implementation of a computer system 700 that may be used in connection with any of the embodiments of the invention described herein is shown in FIG. 4. The computer system 700 may include one or more processors 710 and one or more computer-readable tangible non-transitory storage media (e.g., memory 720, one or more non-volatile storage media 730, or any other suitable storage device). The processor 710 may control writing data to and reading data from the memory 720 and the non-volatile storage device 730 in any suitable manner, as the aspects of the present invention described herein are not limited in this respect. To perform any of the functionality described herein, the processor 710 may execute one or more instructions stored in one or more computer-readable storage media (e.g., the memory 720), which may serve as tangible non-transitory computer-readable storage media storing instructions for execution by the processor 710.

According to some aspects of the invention, methods to treat prostate tumor are provided. In some embodiments, the methods comprise obtaining a prostate tumor sample from a subject; measuring a metabolic profile of the tumor sample, wherein the metabolites are differentially produced in prostate tumors with high Akt1 expression versus prostate tumors with high Myc expression; comparing the metabolic profile to an appropriate reference profile of the metabolites; and treating the subject with an Akt1 inhibitor when results of the comparison of the metabolic profile indicate high Akt1 expression in the tumor sample and/or treating the subject with a Myc inhibitor when results of the comparison of the metabolic profile indicate high Myc in the tumor sample.

In some embodiments, the method to treat prostate tumor comprises obtaining a biological sample from a subject; measuring a level of sarcosine in the sample; comparing the level of sarcosine in the sample to a control sarcosine level; and treating the subject with a Myc inhibitor when the measured level of sarcosine in the sample is increased relative to the control level.

Sarcosine, also known as N-methylglycine, is an intermediate and byproduct in glycine synthesis and degradation. Sarcosine is metabolized to glycine by the enzyme sarcosine dehydrogenase, while glycine-N-methyl transferase generates sarcosine from glycine. In some embodiments, the level of sarcosine in the sample is measured using one or more of mass spectroscopy, nuclear magnetic resonance or chromatography. As described herein, the biological sample includes, but is not limited to urine, blood, serum, plasma, and tissue.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An Akt1 inhibitor includes, but is not limited to (a) a low molecular weight compound or high molecular weight compound which inhibits the phosphorylation of Akt1, (b) a low molecular weight compound or high molecular weight compound which inhibits the expression of Akt1, (c) an antibody which inhibits the phosphorylation of Akt1, (d) an antibody which inhibits the expression of Akt1, (e) a siRNA or shRNA against a polynucleotide encoding Akt1, (f) an antisense polynucleotide comprising a nucleotide sequence complementary or substantially complementary to the nucleotide sequence of a polynucleotide encoding Akt1, or comprising a part of said nucleotide sequence, (g) a ribozyme directed to a polynucleotide encoding Akt1, (h) a mutant of Akt1 which dominant-negatively acts on Akt1 or a polynucleotide encoding said mutant, and (i) an aptamer against Akt1. In some embodiments, the Akt1 inhibitor is Perifosine, Miltefosine, MK2206 (Hirai et al. Mol Cancer Ther. 2010 July; 9(7):1956-67), GSK690693 (Rhodes et al. Cancer Res Apr. 1, 2008 68; 2366), GDC-0068 (Saura et al. J Clin Oncol 30, 2012 (suppl; abstr 3021), or AZD5363 (Davies et al. (Mol Cancer Ther. 2012 April; 11(4):873-87).

A Myc inhibitor includes, but is not limited to (a) a low molecular weight compound or high molecular weight compound which inhibits the expression of Myc, (b) an antibody which inhibits the expression of Myc, (e) a siRNA or shRNA against a polynucleotide encoding Myc, (f) an antisense polynucleotide comprising a nucleotide sequence complementary or substantially complementary to the nucleotide sequence of a polynucleotide encoding Myc, or comprising a part of said nucleotide sequence, (g) a ribozyme directed to a polynucleotide encoding Myc, (h) a mutant of Myc which dominant-negatively acts on Myc or a polynucleotide encoding said mutant, and (i) an aptamer against Myc. In some embodiments, the Myc inhibitor is selected from the group consisting of 10058-F4 (Huang et al. Exp Hematol. 2006 November; 34(11):1480-9.), JQ1 (Delmore et al. Cell. 2011 Sep. 16; 146(6):904-17) and Omomyc (Soucek et al. Cancer Res Jun. 15, 2002 62; 3507).

The inhibitors described herein are administered in effective amounts. An effective amount is a dose sufficient to provide a medically desirable result and can be determined by one of skill in the art using routine methods. In some embodiments, an effective amount is an amount which results in any improvement in the condition being treated. In some embodiments, an effective amount may depend on the type and extent of cancer being treated and/or use of one or more additional therapeutic agents. However, one of skill in the art can determine appropriate doses and ranges of inhibitors to use, for example based on in vitro and/or in vivo testing and/or other knowledge of compound dosages. When administered to a subject, effective amounts will depend, of course, on the particular tumor being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

In the treatment of prostate tumor, an effective amount will be that amount which shrinks cancerous tissue (e.g., tumor), produces a remission, prevents further growth of the tumor and/or reduces the likelihood that the cancer in its early stages (in situ or invasive) does not progress further to metastatic prostate cancer. An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several days (depending of course of the mode of administration and the factors discussed above).

Actual dosage levels can be varied to obtain an amount that is effective to achieve the desired therapeutic response for a particular patient, compositions, and mode of administration. The selected dosage level depends upon the activity of the particular compound, the route of administration, the severity of the tumor, the tissue being treated, and prior medical history of the patient being treated. However, it is within the skill of the art to start doses of the compound at levels lower than required to achieve the desired therapeutic effort and to gradually increase the dosage until the desired effect is achieved.

The Akt1 and/or Myc inhibitors and pharmaceutical compositions containing these compounds are administered to a subject by any suitable route. For example, the inhibitors can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the prostate. In some embodiments, the compositions may be administered systemically.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Methods

### Generation of AKT1- and MYC-Overexpressing RWPE-1

Immortalized human prostate epithelial RWPE-1 cells were infected with pBABE retroviral constructs of myristoylated AKT1 (RW-AKT1) or MYC (RW-MYC), containing a puromycin resistance gene. Infection of pBABE vector alone (RW-EV) was used as a control. Cells were transduced through infection in the presence of polybrene (8 μg/mL), and retroviral supernatants were replaced with fresh media after 4 hours of incubation. Twenty-four hours later, Puromycin selection (1 μg/mL) was started. Cells were grown in phenol red-free Minimum Essential Media (MEM) supplemented with 10% Fetal Bovine Serum (FBS), 0.1 mM non-essential amino acids, 1 mM sodium pyruvate and penicillin-streptomycin (Gibco, Life Technologies).

### Transgenic Mice

Ventral prostate lobes were isolated from 13 week-old MPAKT (4) and Lo-Myc (5) transgenic mice and from age-matched wild-type mice (FVB strain) within 10 minutes after CO₂ euthanasia. Tissues were snap-frozen in isopropanol cooled with dry ice immediately following harvest and stored at −80° C. until metabolite extraction.

### Human Prostate Tissues

Fresh-frozen, optimal cutting temperature (OCT) compound-embedded radical prostatectomy samples were obtained from the Institutional tissue repository at the Dana-Farber Cancer Institute/Brigham and Women's Hospital (40 tumors and 21 normals) and from an independent collection of archival tissues (21 tumors and 4 normals; Dana-Farber Cancer Institute). All samples were collected with informed consent approved by the Institutional Review Board.

The presence and percentage of tumor was assessed in each tissue sample on frozen sections. One case was excluded from the study because of no tumor evidence. DNA, RNA and proteins were purified from serial 8 μm sections of each OCT-embedded tissue block. The remaining tissue was processed for metabolite extraction.

### Metabolite Profiling

Metabolite profiling analysis was performed by Metabolon Inc. (Durham, N. C.) as previously described (Evans, A. M., DeHaven, C. D., Barrett, T., Mitchell, M. & Milgram, E. Integrated, nontargeted ultrahigh performance liquid chromatography/electrospray ionization tandem mass spectrometry platform for the identification and relative quantification of the small-molecule complement of biological systems. Anal Chem 81, 6656-6667 (2009); Sha, W., et al. Metabolomic profiling can predict which humans will develop liver dysfunction when deprived of dietary choline. FASEB J 24, 2962-2975 (2010)).

### Sample Accessioning.

Each sample received was accessioned into the Metabolon LIMS system and was assigned by the LIMS a unique identifier that was associated with the original source identifier only. This identifier was used to track all sample handling, tasks, results etc. The samples (and all derived aliquots) were tracked by the LIMS system. All portions of any sample were automatically assigned their own unique identifiers by the LIMS when a new task is created; the relationship of these samples is also tracked. All samples were maintained at −80° C. until processed.

### Sample Preparation.

Samples were prepared using the automated MicroLab STAR® system (Hamilton Robotics, Inc., NV). A recovery standard was added prior to the first step in the extraction process for QC purposes. Sample preparation was conducted using aqueous methanol extraction process to remove the protein fraction while allowing maximum recovery of small molecules. The resulting extract was divided into four fractions: one for analysis by UPLC/MS/MS (positive mode), one for UPLC/MS/MS (negative mode), one for GC/MS, and one for backup. Samples were placed briefly on a TurboVap® (Zymark) to remove the organic solvent. Each sample was then frozen and dried under vacuum. Samples were then prepared for the appropriate instrument, either UPLC/MS/MS or GC/MS.

### Ultrahigh Performance Liquid Chromatography/Mass Spectroscopy (UPLC/MS/MS).

The LC/MS portion of the platform was based on a Waters ACQUITY ultra-performance liquid chromatography (UPLC) and a Thermo-Finnigan linear trap quadrupole (LTQ) mass spectrometer, which consisted of an electrospray ionization (ESI) source and linear ion-trap (LIT) mass analyzer. The sample extract was dried then reconstituted in acidic or basic LC-compatible solvents, each of which contained 8 or more injection standards at fixed concentrations to ensure injection and chromatographic consistency. One aliquot was analyzed using acidic positive ion optimized conditions and the other using basic negative ion optimized conditions in two independent injections using separate dedicated columns. Extracts reconstituted in acidic conditions were gradient eluted using water and methanol containing 0.1% formic acid, while the basic extracts, which also used water/methanol, contained 6.5 mM Ammonium Bicarbonate. The MS analysis alternated between MS and data-dependent MS² scans using dynamic exclusion. Raw data files are archived and extracted as described below.

### Gas Chromatography/Mass Spectroscopy (GC/MS).

The samples destined for GC/MS analysis were re-dried under vacuum desiccation for a minimum of 24 hours prior to being derivatized under dried nitrogen using bistrimethyl-silyl-triflouroacetamide (BSTFA). The GC column was 5% phenyl and the temperature ramp was from 40° to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis. The information output from the raw data files was automatically extracted as discussed below.

### Quality Assurance/QC.

For QA/QC purposes, additional samples were included with each day's analysis. These samples included extracts of a pool of well-characterized human plasma, extracts of a pool created from a small aliquot of the experimental samples, and process blanks. QC samples were spaced evenly among the injections and all experimental samples were randomly distributed throughout the run. A selection of QC compounds was added to every sample for chromatographic alignment, including those under test. These compounds were carefully chosen so as not to interfere with the measurement of the endogenous compounds.

### Data Extraction and Compound Identification.

Raw data was extracted, peak-identified and QC processed using Metabolon's hardware and software. These systems are built on a web-service platform utilizing Microsoft's .NET technologies, which run on high-performance application servers and fiber-channel storage arrays in clusters to provide active failover and load-balancing (Dehaven, C. D., Evans, A. M., Dai, H. & Lawton, K. A. Organization of GC/MS and LC/MS metabolomics data into chemical libraries. J Cheminform 2, 9 (2010)). Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, nominal mass match to the library +/−0.2 amu (atomic mass units), and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 2400 commercially available purified standard compounds have been acquired and registered into LIMS for distribution to both the LC and GC platforms for determination of their analytical characteristics.

### Data Analysis:

For studies spanning multiple days, a data normalization step is performed to correct variation resulting from instrument inter-day tuning differences. Essentially, each compound is corrected in run-day blocks by registering the medians to equal one (1.00) and normalizing each data point proportionately (termed the “block correction”). For studies that do not require more than one day of analysis, no normalization is necessary, other than for purposes of data visualization. Second, for each sample, metabolite values are normalized by cell count (cell lines) or tissue weight (mouse or human prostate tissue). Third, median scaling of each metabolite across all samples and imputation of each metabolite by the minimum observed value of that compound were performed. Finally, quantile normalization of every sample was applied to ensure statistically comparable distributions. To obtain differential metabolites across 3 classes, MYC-high, phosphoAKT-high and control, we used the one class-versus-all permutation based t test, as implemented in GenePattern (Reich, M., et al. GenePattern 2.0. Nat Genet 38, 500-501 (2006)) to identify compounds associated with MYC or AKT overexpression. A p-value threshold of 0.05 was used to determine the significant compounds. GeneSet Enrichment Analysis (GSEA) (Subramanian, A., et al. Gene set enrichment analysis: a knowledge-based approach for interpreting genome-wide expression profiles. Proc Natl Acad Sci USA 102, 15545-15550 (2005)) was used to measure the enrichment of KEGG defined pathways²³ both within (i) individual samples and (ii) across MYC-high and AKT-high samples, as previously described (Subramanian, A., et al. Gene set enrichment analysis: a knowledge-based approach for interpreting genome-wide expression profiles. Proc Natl Acad Sci USA 102, 15545-15550 (2005); Barbie, D. A., et al. Systematic RNA interference reveals that oncogenic KRAS-driven cancers require TBK1. Nature 462, 108-112 (2009). Gene set-size-normalized enrichment scores (NES) from GSEA were used to determine the extent and direction of enrichment for each pathway in different systems that were represented by at least 2 metabolites. The mean NES of the 3 systems was computed for each pathway and the pathways that are consistently enriched across all systems were detected as outliers using box-and-whisker plots (with 75% or more times the inter-quartile range from the box).

### Single Nucleotide Polymorphisms (SNP) Arrays

Two-hundred-fifty ng of DNA extracted from 60 prostate tumors and 6 matched normal tissue samples were labeled and hybridized to the Affymetrix 250K Sty I array to obtain signal intensities and genotype calls (Microarray core facility, Dana-Farber Cancer Institute). Signal intensities were normalized against data from normal samples. Copy-number profiles were inferred and the significance of somatic copy-number alterations was determined using the GISTIC module in GenePattern. The heat map was generated using DChip 2010.01 (http://biosunl.harvard.edu/complab/dchip/download.htm).

mRNA Expression Analysis

Total RNA was isolated from RWPE-EV, RWPE-AKT1 and RWPE-MYC cells (RNeasy Micro Kit, Qiagen Inc., CA) and from the prostate tumors and matched normal controls (AllPrep DNA/RNA Micro Kit, Qiagen Inc.). Two micrograms of RNA from each isogenic cell line were retro-transcribed with the SuperScript™ First-Strand Synthesis System (Invitrogen, Life Technologies Corporation, NY), and 5 ng of cDNA were used per each gene expression reaction with the specific TaqMan probe (Applied Biosystems). For the human prostate tissues, 300-400 ng of purified RNA were retro-transcribed using High Capacity cDNA Reverse transcription kit (Applied Biosystems). One hundred ng of cDNA was used to perform relative real time PCR using custom micro fluidic cards (Taqman Custom Arrays, Applied Biosystems) and Applied Biosystems 7900 HT Fast Real-Time System, as described by the manufacturer. All samples were run in duplicate and normalized to the average of actin, gus and 18S rRNA, which have stable expression in our experimental conditions. Data were analyzed using the ΔΔCt method and obtained values were expressed as n-fold the calibrator (RWPE-1 cells or the average of 8 normal prostate tissues) set as 1. Probes and primers included in the fluidic card were purchased from Applied Biosystems. One-sample T-Test was applied and significance was defined with p<0.05.

### Results:

To profile the metabolic heterogeneity of prostate cancer in an oncogene-specific context, phosphorylated AKT1- or MYC-associated metabolomic signatures from prostate epithelial cells in monolayer culture, transgenic mouse prostate and primary nonmetastatic prostate tumors were integrated. The aim was to identify patterns of metabolomic changes that were different for the two oncogenes but common for the three biological systems.

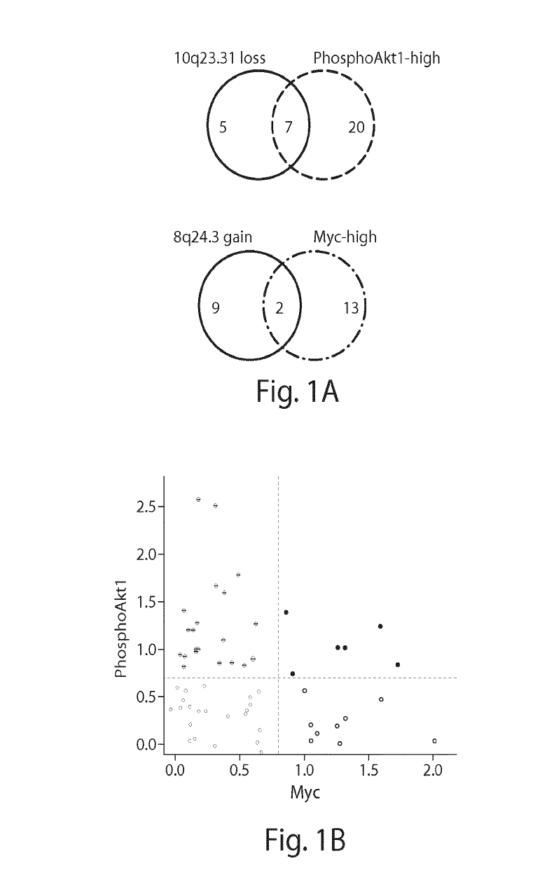

First, it was determined whether genomic alterations at the PTEN or MYC loci would be predictive of active AKT1 or MYC overexpression in a cohort of 60 prostate tumors obtained from the Institutional Tissue Repository. These tumors were pathological stage T2, 22 high Gleason (4+3 or 4+4) and 38 low Gleason (3+3 or 3+4). Genomic DNA and proteins extracted from sections of each tumor or nontumoral matched control sample were assayed by Single Nucleotide Polymorphisms (SNP) arrays and western blotting (phosphorylated AKT1 and MYC). SNP arrays revealed that 20% of these tumors harbored 10q loss and 18% harbored 8q gain. K-means clustering of phosphorylated AKT1 and MYC western blot densitometric values was conducted in parallel to segregate 4 prostate tumor subgroups, i.e. phosphoAKT1-high/MYC-high, phosphoAKT1-high/MYC-low, phosphoAKT1-low/MYC-high and phosphoAKT1-low/MYC-low (FIG. 1B). Importantly, the genomic alterations only counted for 7/27 (26%) of phosphoAKT1-high tumors and for 2/15 (13%) of MYC-high tumors, suggesting the protein signature to be the most accurate to assess activation of AKT1 or MYC (FIG. 1A). In addition, levels of phosphoAKT1 and MYC were not associated with the Gleason grade of the tumors.

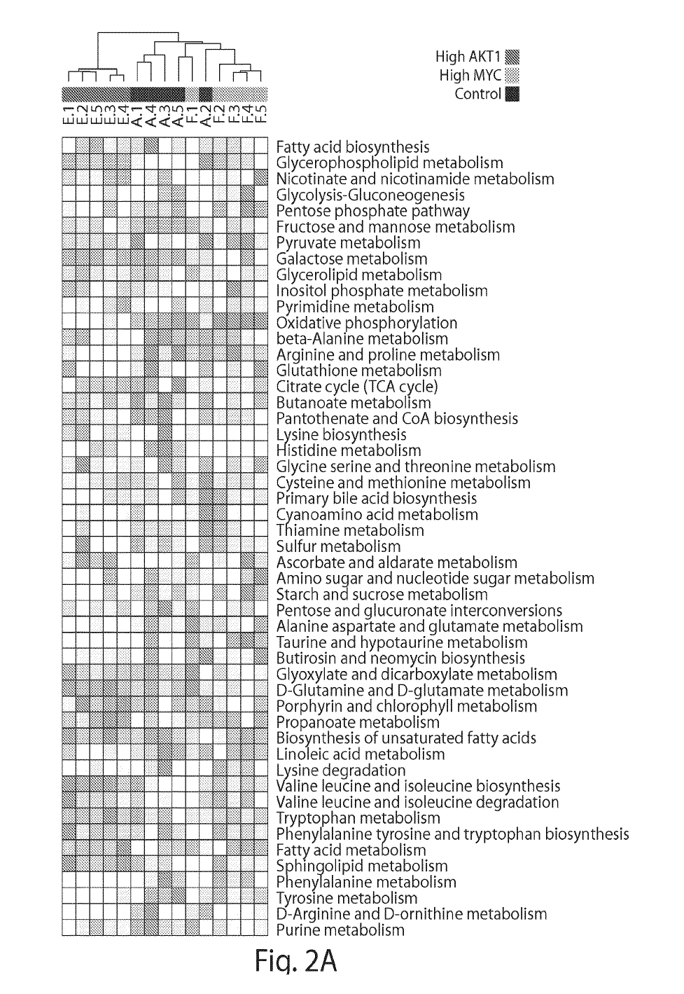

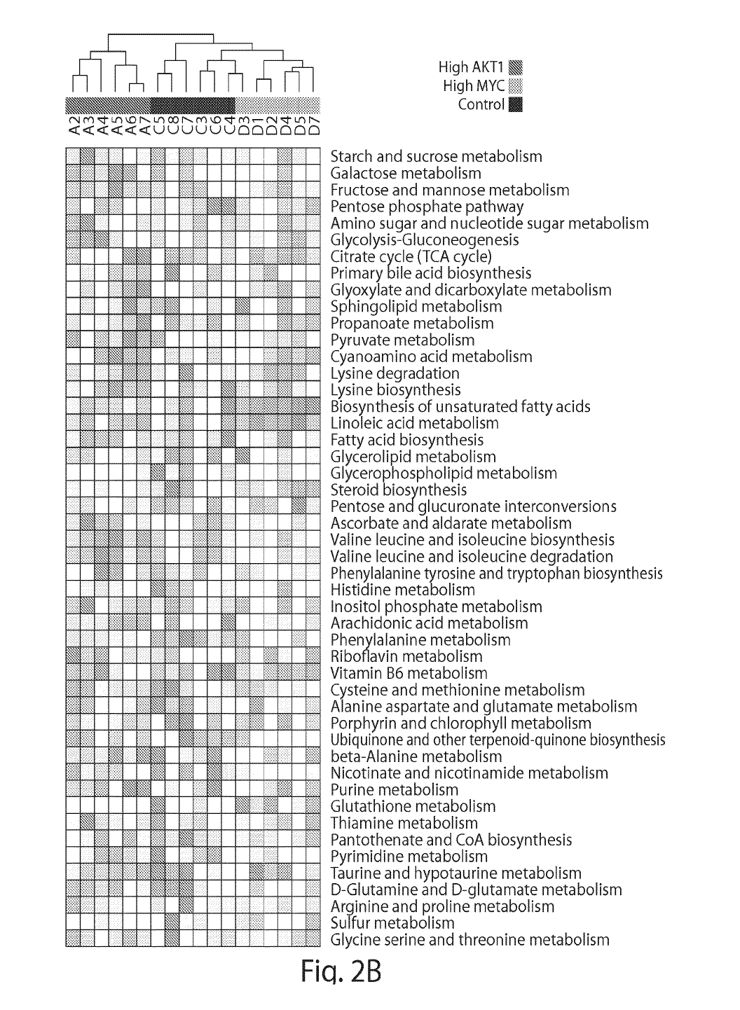

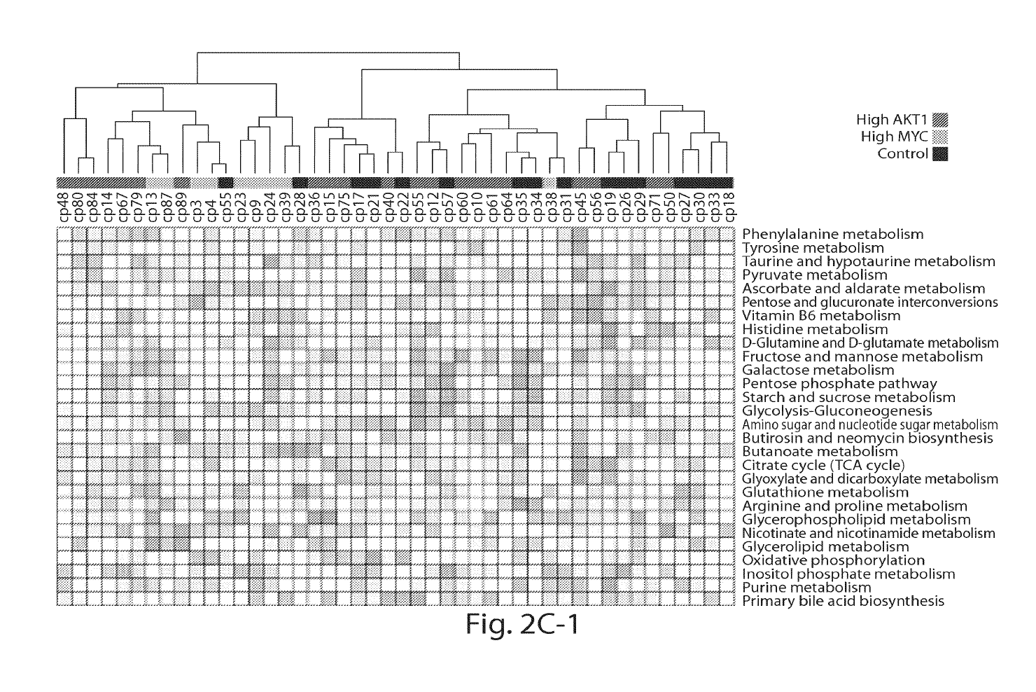

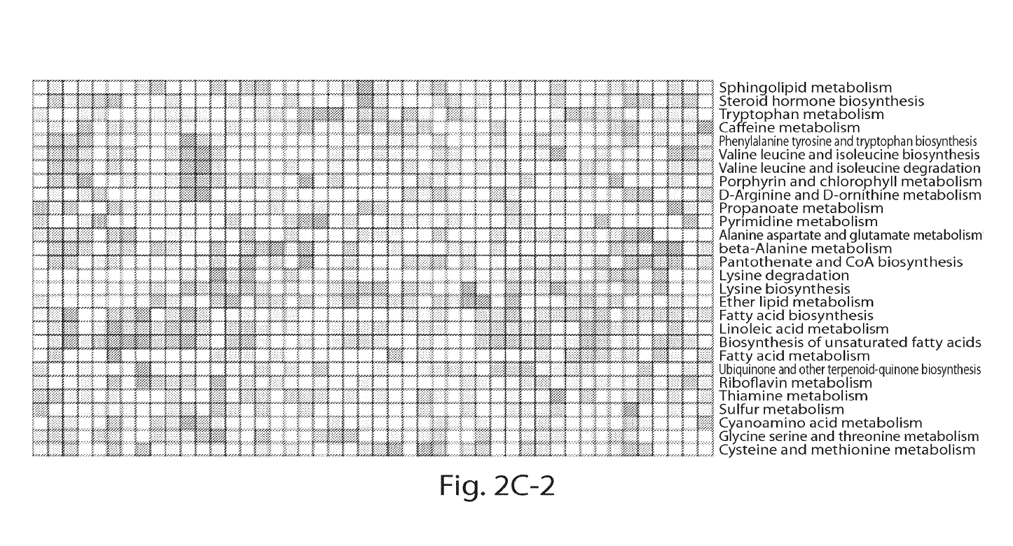

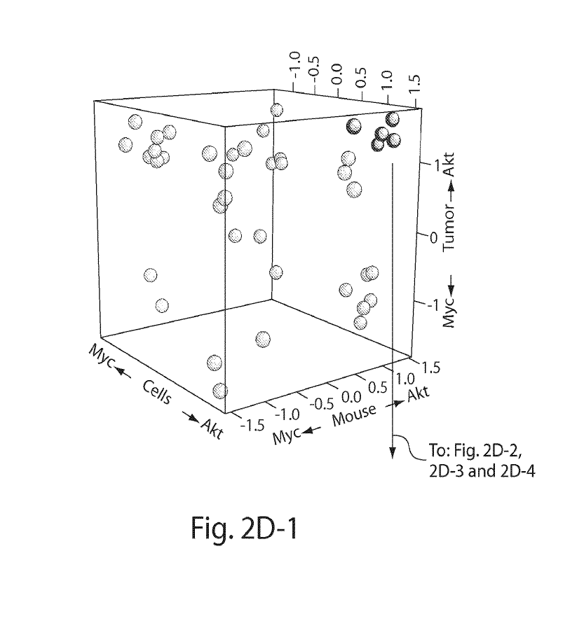

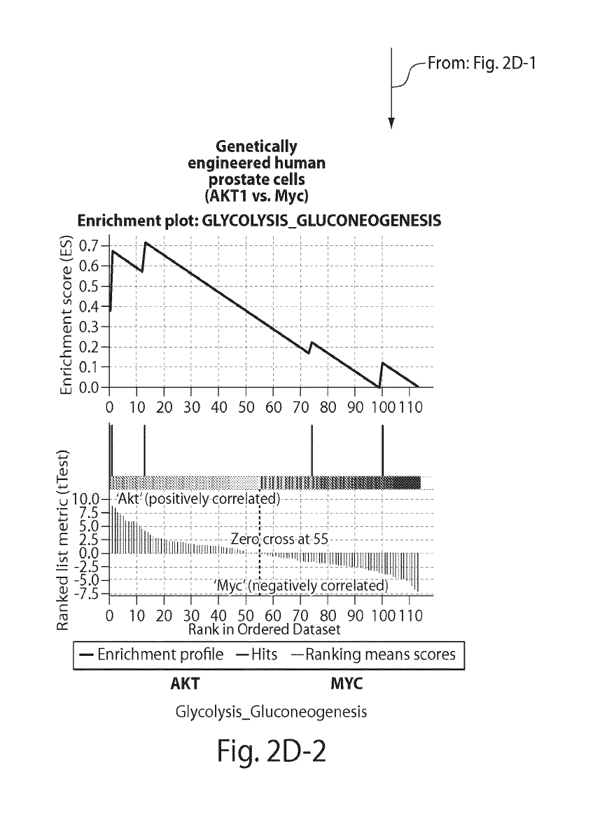

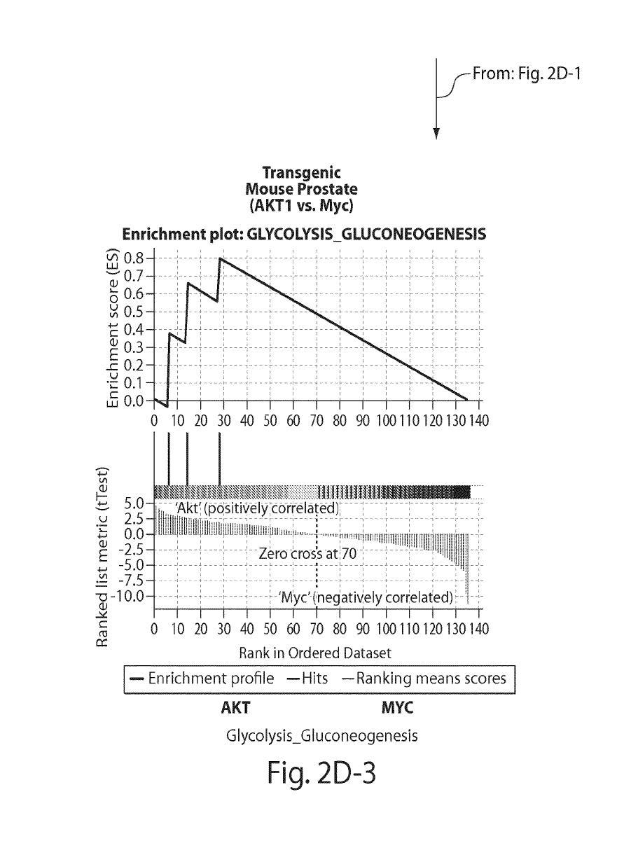

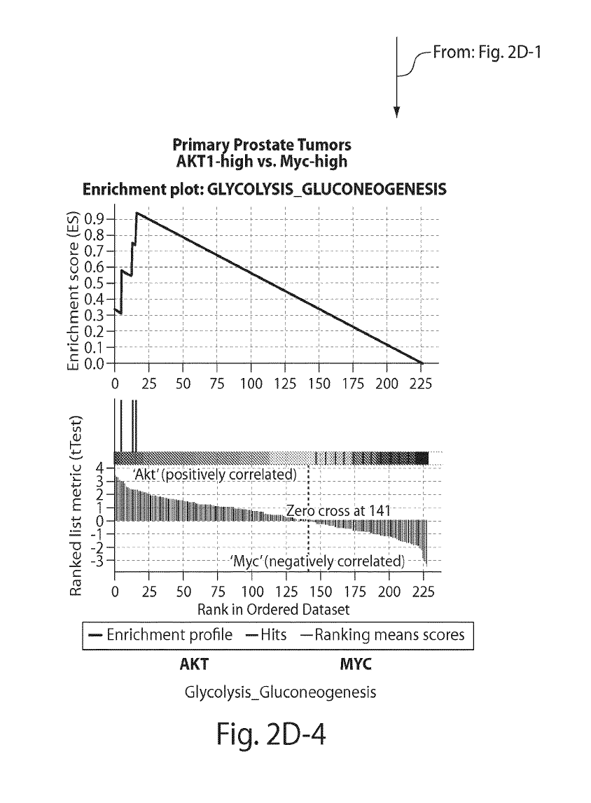

Next, to define differential metabolic reprogramming induced by sole activation of AKT1 or overexpression of MYC in non-transformed prostate, mass-spectrometry based metabolomics of prostate epithelial RWPE-1 cells genetically engineered with constructs encoding myristoylated AKT1 or MYC, and transgenic mice expressing human myristoylated AKT1 or MYC in the prostate was performed. Interestingly, while both RW-AKT1 and RW-MYC cells showed significant changes in intermediates of glycolysis, only RW-AKT1 cells exhibited the aerobic glycolytic phenotype (FIG. 2A). These results were even more pronounced in vivo (FIG. 2B and FIG. 2C), with exclusively the MPAKT transgenic mouse prostate being characterized by both very high levels of glucose metabolism intermediates and lactate (FIG. 2B). In turn, MYC overexpression was associated with a distinctive signature of lipids, including enrichment of metabolites sets of unsaturated fatty acids both in transgenic mouse prostate and in human tumors. When applied to primary non-metastatic prostate tumors stratified by the expression levels of phospho-AKT1 and MYC, the pathway enrichment analysis revealed that MYC-high tumors rather show a negative enrichment of glycolysis compared to phosphoAKT1-high and nontumoral prostate tissue (FIG. 2C).

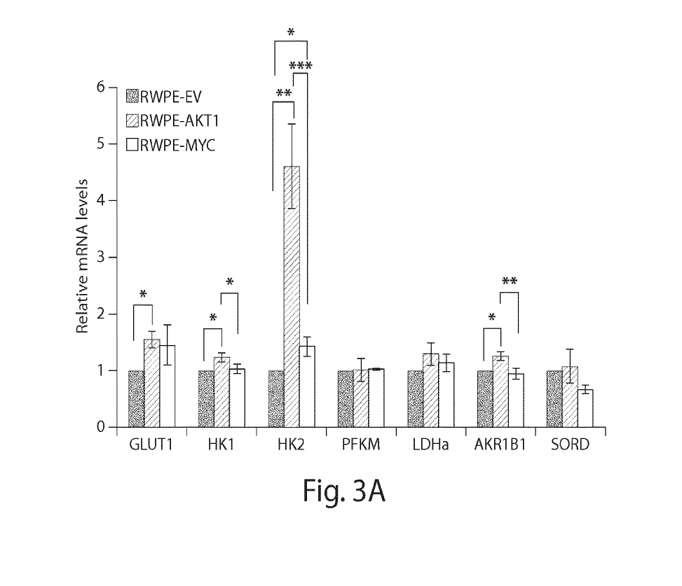

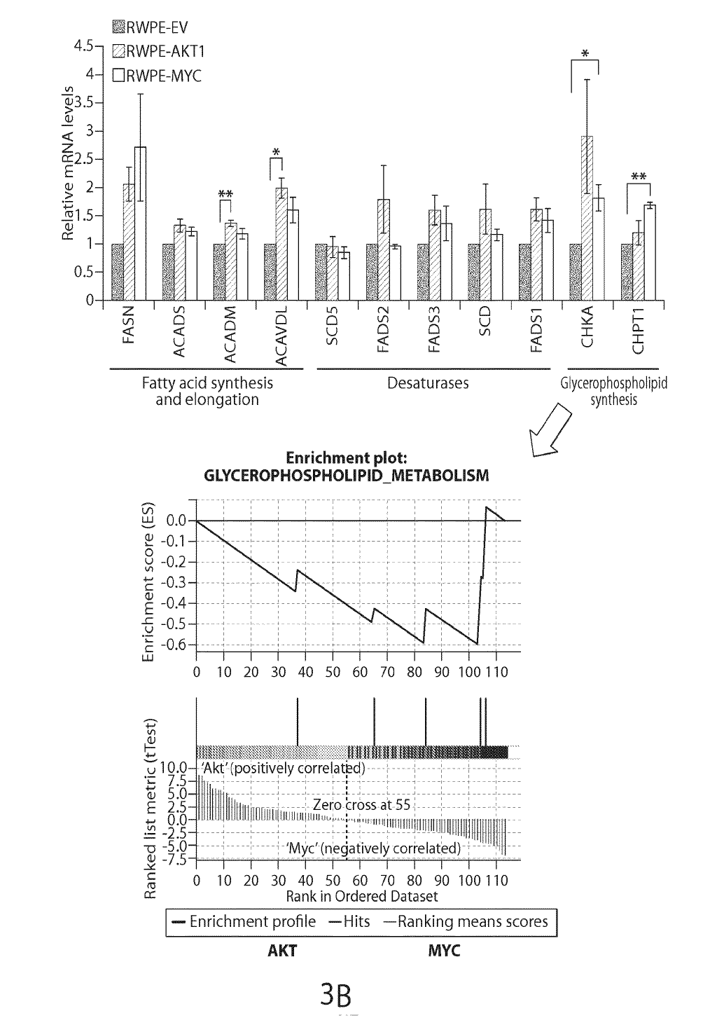

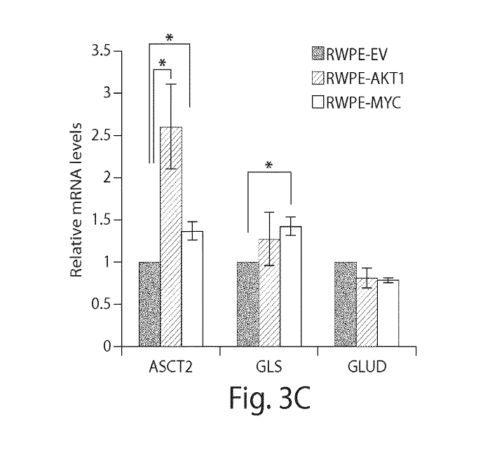

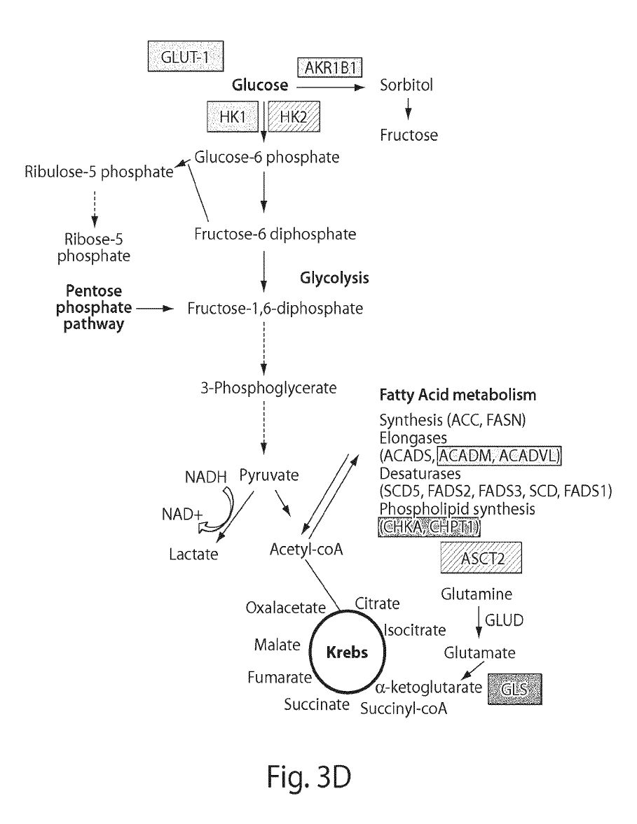

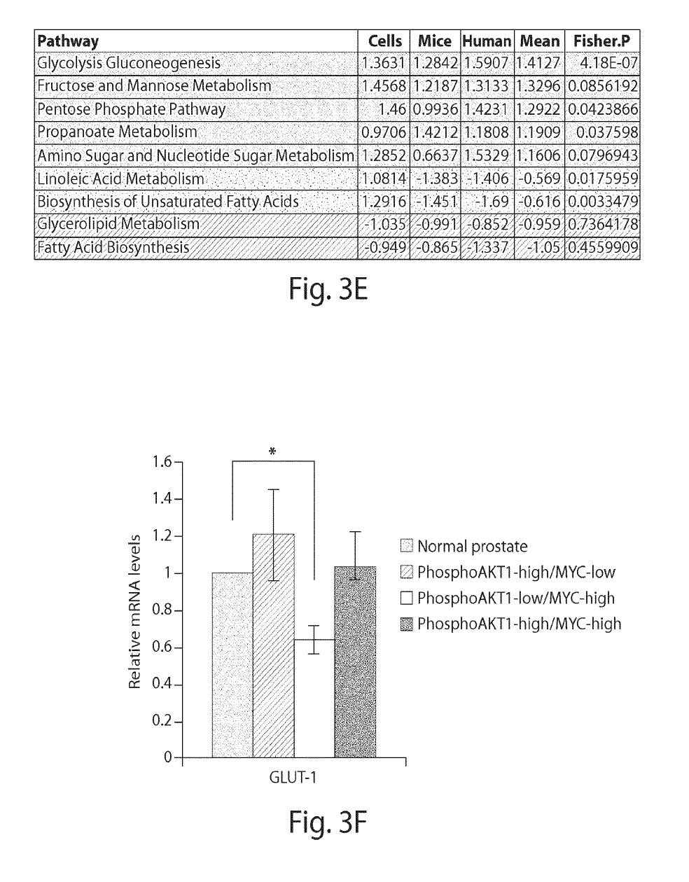

Next, the AKT1 and MYC metabolic signatures were compared directly. The list of metabolites with fold changes and p-values (phosphoAKT1-high vs. MYC-high) per data set (RWPE cells, probasin-driven transgenic mice and prostate tumors) is given in the Table 2. Pathway enrichment analysis by GSEA was used to determine which metabolic pathways were commonly enriched in the genetically engineered models and in the prostate tumor subgroups defined above, specifically comparing high AKT1 with high MYC background (FIG. 2D). Complete lists of the metabolite sets tested, the number of metabolites per set, and the enrichment scores are included in the Table 3. In detail, gene set-size-normalized enrichment scores (NES) from GSEA were used to determine the extent and direction of enrichment for each pathway in the 3 data sets. Five pathways with highly positive NES and 2 pathways with highly negative NES across biological systems were defined as outliers (FIG. 2D and FIG. 3E). This analysis showed that AKT1 exquisitely drives aerobic glycolysis and other glucose-related pathways such as the pentose phosphate shunt and fructose metabolism, whereas MYC is a promoter of lipid metabolism (FIG. 3E). On the one hand, enrichment of the glycerophospholipid, glycerolipid and pantothenate/coA biosynthesis metabolite sets, as well as higher levels of lipogenesis-feeding metabolites such as citrate, were distinctively associated with MYC overexpression in RWPE cells, suggesting that MYC induces synthesis and/or turnover of membrane lipids. This would be justified by the higher proliferation requirement of these cells. On the other hand, it was intriguing to find higher levels of omega-3 (docosapentaenoate and docosahexaenoate) and omega-6 (arachidonate, docosadienoate and dihomo-linolenate) fatty acids in the ventral prostate of Lo-MYC mice and in MYC-high/phosphoAKT1-low prostate tumors relative to MPAKT mice and phosphoAKT1-high/MYC-low tumors, respectively (FIG. 3E). These are essential fatty acids, therefore obtained from extracellular sources. Although the precise role of these unsaturated fatty acids in prostate cancer is not completely understood, the data reveals that prostate cells may increase their lipid needs early during transformation, as seen in Low-MYC mice. One possibility would be that these lipids are used as energy sources via oxidation.

Finally, it was determined whether the metabolome changes associated with the oncogenic transformation of prostate epithelial cells are accompanied by transcriptional changes in key metabolic enzymes. Consistent with the metabolite profiling of RWPE-1 cells, glycolytic enzymes such as the glucose transporter GLUT-1, the hexokinases 1 and 2, and the aldose reductase AKR1B1 were significantly increased upon AKT1 overexpression/activation (FIG. 3A, 3D), whereas only a moderate increase in hexokinase 2 occurred in RWPE-MYC cells. When looking at lipogenic enzymes, instead, two key enzymes of the glycerophospholipid metabolism, choline kinase and cholinephosphotransferase-1, were both induced by MYC overexpression (FIG. 3B,3D), validating the enrichment of the glycerophospholipid metabolic set in RWPE-MYC cells (FIG. 3B). The glutamine pathway was also affected by the activation/overexpression of AKT1 and MYC. While both oncogenes increased the mRNA levels of the neutral amino acid transporter ASCT2, only MYC significantly induced glutaminase, the glutaminolytic enzyme responsible for the conversion of glutamine into glutamate (FIG. 3C, 3D). In addition, sarcosine, an intermediate of the glycine and choline metabolism previously identified as a progression marker in prostate cancer, increased exclusively in the prostate of Lo-MYC mice. Associated with the sarcosine increase were a concomitant elevation of the intermediate betaine and a decrease in glycine levels. These results suggest a dysregulation of the sarcosine pathway upon MYC overexpression.

To identify unique mRNA expression changes in phosphoAKT1-low/MYC-high (n=5) and phosphoAKT1-low/MYC-high (n=13) prostate tumors, a qPCR-based expression profiling analysis was performed of 29 metabolic genes in the 2 tumor groups relative to normal prostate tissues (n=8). Consistent with the metabolomics results, high MYC expression in a phosphoAKT1-low context in human tumors was associated with decreased mRNA expression of the glucose transporter-1 (GLUT-1) (FIG. 3D, 3F). No decrease in GLUT-1 expression was found in phosphoAKT1-high/MYC-high tumors (n=3) (FIG. 4e). Altogether, these results suggest that MYC activation affects glucose uptake and glucose utilization rate in prostate tumors.

In summary, the data demonstrates that individual prostate tumors have distinct metabolic phenotypes resulting from their genetic complexity, and reveal a novel metabolic role for MYC in prostate cancer. The evidence that MYC overexpression inversely associates with GLUT-1 mRNA expression and with the AKT1-dependent “Warburg effect” metabolic phenotype in transformed prostate cells opens novel avenues for the metabolic imaging of prostate cancer patients whose tumors harbor 8q amplification or PTEN loss and/or show MYC or AKT1 activation. Through large-scale metabolite analyses and isotopic labeling approaches, as well as generation of metabolic set enrichment pathways, it was found that AKT1 drives primarily aerobic glycolysis while MYC does not elicit a Warburg-like effect and significantly enhances glycerophospholipid synthesis instead. This regulation is Gleason grade- and pathological stage-independent. These results demonstrates that human prostate tumors exhibit metabolic fingerprints of their molecular phenotypes, which may have impact on metabolic diagnostics and targeted therapeutics.

TABLE 1
List of metabolites tested.

| Id | Compound | KEGG_Id | Family | Pathway |
|---|---|---|---|---|
| M37180 | 2 amino p cresol sulfate | NA | Amino acid | Phenylalanine and tyrosine metabolism |
| M1126 | alanine | C00041 | Amino_acid | Alanine_and_aspartate_metabolism |
| M11398 | asparagine | C00152 | Amino_acid | Alanine_and_aspartate_metabolism |
| M1585 | N-acetylalanine | C02847 | Amino_acid | Alanine_and_aspartate_metabolism |
| M15996 | aspartate | C00049 | Amino_acid | Alanine_and_aspartate_metabolism |
| M22185 | N-acetylaspartate | C01042 | Amino_acid | Alanine_and_aspartate_metabolism |
| M3155 | 3-ureidopropionate | C02642 | Amino_acid | Alanine_and_aspartate_metabolism |
| M443 | aspartate | C00049 | Amino_acid | Alanine_and_aspartate_metabolism |
| M55 | beta-alanine | C00099 | Amino_acid | Alanine_and_aspartate_metabolism |
| M1577 | 2-aminobutyrate | C02261 | Amino_acid | Butanoate_metabolism |
| M27718 | creatine | C00300 | Amino_acid | Creatine_metabolism |
| M513 | creatinine | C00791 | Amino_acid | Creatine_metabolism |
| M1302 | methionine | C00073 | Amino_acid | Cysteine,_methionine,_SAM,_taurine_metabolism |
| M15705 | cystathionine | C02291 | Amino_acid | Cysteine,_methionine,_SAM,_taurine_metabolism |
| M1589 | N-acetylmethionine | C02712 | Amino_acid | Cysteine,_methionine,_SAM,_taurine_metabolism |
| M15948 | S-adenosylhomocysteine | C00021 | Amino_acid | Cysteine,_methionine,_SAM,_taurine_metabolism |
| M21044 | 2-hydroxybutyrate | C05984 | Amino_acid | Cysteine,_methionine,_SAM,_taurine_metabolism |
| M2125 | taurine | C00245 | Amino_acid | Cysteine,_methionine,_SAM,_taurine_metabolism |
| M31453 | cysteine | C00097 | Amino_acid | Cysteine,_methionine,_SAM,_taurine_metabolism |
| M31454 | cystine | C00491 | Amino_acid | Cysteine,_methionine,_SAM,_taurine_metabolism |
| M590 | hypotaurine | C00519 | Amino_acid | Cysteine,_methionine,_SAM,_taurine_metabolism |
| M1416 | gamma-aminobutyrate | C00334 | Amino_acid | Glutamate_metabolism |
| M1647 | glutamine | C00064 | Amino_acid | Glutamate_metabolism |
| M32672 | pyroglutamine | NA | Amino_acid | Glutamate_metabolism |
| M33487 | glutamate, gamma-methyl ester | NA | Amino_acid | Glutamate_metabolism |
| M33943 | N-acetylglutamine | C02716 | Amino_acid | Glutamate_metabolism |
| M35665 | N-acetyl-aspartyl-glutamate | C12270 | Amino_acid | Glutamate_metabolism |
| M53 | glutamine | C00064 | Amino_acid | Glutamate_metabolism |
| M57 | glutamate | C00025 | Amino_acid | Glutamate_metabolism |
| M1494 | 5-oxoproline | C01879 | Amino_acid | Glutathione_metabolism |
| M15731 | S-lactoylglutathione | C03451 | Amino_acid | Glutathione_metabolism |
| M2127 | glutathione, reduced | C00051 | Amino_acid | Glutathione_metabolism |
| M27727 | glutathione, oxidized | C00127 | Amino_acid | Glutathione_metabolism |
| M33016 | ophthalmate | NA | Amino_acid | Glutathione_metabolism |
| M34592 | ophthalmate | NA | Amino_acid | Glutathione_metabolism |
| M35159 | cysteine-glutathione disulfide | NA | Amino_acid | Glutathione_metabolism |
| M11777 | glycine | C00037 | Amino_acid | Glycine,_serine_and_threonine_metabolism |
| M1284 | threonine | C00188 | Amino_acid | Glycine,_serine_and_threonine_metabolism |
| M1516 | sarcosine | C00213 | Amino_acid | Glycine,_serine_and_threonine_metabolism |
| M1648 | serine | C00065 | Amino_acid | Glycine,_serine_and_threonine_metabolism |
| M3141 | betaine | C00719 | Amino_acid | Glycine,_serine_and_threonine_metabolism |
| M33939 | N-acetylthreonine | C01118 | Amino_acid | Glycine,_serine_and_threonine_metabolism |
| M37076 | N-acetylserine | NA | Amino_acid | Glycine,_serine_and_threonine_metabolism |
| M15681 | 4-guanidinobutanoate | C01035 | Amino_acid | Guanidino_and_acetamido_metabolism |
| M15677 | 3-methylhistidine | C01152 | Amino_acid | Histidine_metabolism |
| M1574 | histamine | C00388 | Amino_acid | Histidine_metabolism |
| M32350 | 1-methylimidazoleacetate | C05828 | Amino_acid | Histidine_metabolism |
| M59 | histidine | C00135 | Amino_acid | Histidine_metabolism |
| M607 | urocanate | C00785 | Amino_acid | Histidine_metabolism |
| M1301 | lysine | C00047 | Amino_acid | Lysine_metabolism |
| M1444 | pipecolate | C00408 | Amino_acid | Lysine_metabolism |
| M1495 | saccharopine | C00449 | Amino_acid | Lysine_metabolism |
| M35439 | glutaroyl carnitine | NA | Amino_acid | Lysine_metabolism |
| M36752 | N6-acetyllysine | C02727 | Amino_acid | Lysine_metabolism |
| M396 | glutarate | C00489 | Amino_acid | Lysine_metabolism |
| M6146 | 2-aminoadipate | C00956 | Amino_acid | Lysine_metabolism |
| M1299 | tyrosine | C00082 | Amino_acid | Phenylalanine_&_tyrosine_metabolism |
| M32197 | 3-(4-hydroxyphenyl)lactate | C03672 | Amino_acid | Phenylalanine_&_tyrosine_metabolism |
| M32553 | phenol sulfate | C02180 | Amino_acid | Phenylalanine_&_tyrosine_metabolism |
| M33945 | phenylacetylglycine | C05598 | Amino_acid | Phenylalanine_&_tyrosine_metabolism |
| M35126 | phenylacetylglutamine | C05597 | Amino_acid | Phenylalanine_&_tyrosine_metabolism |
| M36103 | p-cresol sulfate | C01468 | Amino_acid | Phenylalanine_&_tyrosine_metabolism |
| M64 | phenylalanine | C00079 | Amino_acid | Phenylalanine_&_tyrosine_metabolism |
| M1408 | putrescine | C00134 | Amino_acid | Polyamine_metabolism |
| M1419 | 5-methylthioadenosine | C00170 | Amino_acid | Polyamine_metabolism |
| M15496 | agmatine | C00179 | Amino_acid | Polyamine_metabolism |
| M37496 | N-acetylputrescine | C02714 | Amino_acid | Polyamine_metabolism |
| M485 | spermidine | C00315 | Amino_acid | Polyamine_metabolism |
| M603 | spermine | C00750 | Amino_acid | Polyamine_metabolism |
| M15140 | kynurenine | C00328 | Amino_acid | Tryptophan_metabolism |
| M18349 | indolelactate | C02043 | Amino_acid | Tryptophan_metabolism |
| M2342 | serotonin | C00780 | Amino_acid | Tryptophan_metabolism |
| M27672 | 3-indoxyl sulfate | NA | Amino_acid | Tryptophan_metabolism |
| M32675 | C-glycosyltryptophan | NA | Amino_acid | Tryptophan_metabolism |
| M33959 | N-acetyltryptophan | C03137 | Amino_acid | Tryptophan_metabolism |
| M37097 | tryptophan betaine | C09213 | Amino_acid | Tryptophan_metabolism |
| M437 | 5-hydroxyindoleacetate | C05635 | Amino_acid | Tryptophan_metabolism |
| M54 | tryptophan | C00078 | Amino_acid | Tryptophan_metabolism |
| M1366 | trans-4-hydroxyproline | C01157 | Amino_acid | Urea_cycle;_arginine-,_proline-,_metabolism |
| M1493 | ornithine | C00077 | Amino_acid | Urea_cycle;_arginine-,_proline-,_metabolism |
| M1638 | arginine | C00062 | Amino_acid | Urea_cycle;_arginine-,_proline-,_metabolism |
| M1670 | urea | C00086 | Amino_acid | Urea_cycle;_arginine-,_proline-,_metabolism |
| M1898 | proline | C00148 | Amino_acid | Urea_cycle;_arginine-,_proline-,_metabolism |
| M2132 | citrulline | C00327 | Amino_acid | Urea_cycle;_arginine-,_proline-,_metabolism |
| M34384 | stachydrine | C10172 | Amino_acid | Urea_cycle;_arginine-,_proline-,_metabolism |
| M36808 | dimethylarginine | C03626 | Amino_acid | Urea_cycle;_arginine-,_proline-,_metabolism |
| M1125 | isoleucine | C00407 | Amino_acid | Valine,_leucine_and_isoleucine_metabolism |
| M12129 | beta-hydroxyisovalerate | NA | Amino_acid | Valine,_leucine_and_isoleucine_metabolism |
| M1649 | valine | C00183 | Amino_acid | Valine,_leucine_and_isoleucine_metabolism |
| M32776 | 2-methylbutyroylcarnitine | NA | Amino_acid | Valine,_leucine_and_isoleucine_metabolism |
| M33441 | isobutyrylcarnitine | NA | Amino_acid | Valine,_leucine_and_isoleucine_metabolism |
| M33937 | alpha-hydroxyisovalerate | NA | Amino_acid | Valine,_leucine_and_isoleucine_metabolism |
| M34407 | isovalerylcarnitine | NA | Amino_acid | Valine,_leucine_and_isoleucine_metabolism |
| M35107 | isovalerylglycine | NA | Amino_acid | Valine,_leucine_and_isoleucine_metabolism |
| M35428 | tiglyl carnitine | NA | Amino_acid | Valine,_leucine_and_isoleucine_metabolism |
| M35431 | 2-methylbutyroylcarnitine | NA | Amino_acid | Valine,_leucine_and_isoleucine_metabolism |
| M35433 | hydroxyisovaleroyl carnitine | NA | Amino_acid | Valine,_leucine_and_isoleucine_metabolism |
| M60 | leucine | C00123 | Amino_acid | Valine,_leucine_and_isoleucine_metabolism |
| M15095 | N-acetylglucosamine | C03878 | Carbohydrate | Aminosugars_metabolism |
| M15096 | N-acetylglucosamine | C00140 | Carbohydrate | Aminosugars_metabolism |
| M15821 | fucose | C00382 | Carbohydrate | Aminosugars_metabolism |
| M1592 | N-acetylneuraminate | C00270 | Carbohydrate | Aminosugars_metabolism |
| M32377 | N-acetylneuraminate | C00270 | Carbohydrate | Aminosugars_metabolism |
| M33477 | erythronate | NA | Carbohydrate | Aminosugars_metabolism |
| M12055 | galactose | C01662 | Carbohydrate | Fructose,_mannose,_galactose,_starch,_and_sucrose_metabolism |
| M1470 | mannose-6-phosphate | C00275 | Carbohydrate | Fructose,_mannose,_galactose,_starch,_and_sucrose_metabolism |
| M15053 | sorbitol | C00794 | Carbohydrate | Fructose,_mannose,_galactose,_starch,_and_sucrose_metabolism |
| M15335 | mannitol | C00392 | Carbohydrate | Fructose,_mannose,_galactose,_starch,_and_sucrose_metabolism |
| M15804 | maltose | C00208 | Carbohydrate | Fructose,_mannose,_galactose,_starch,_and_sucrose_metabolism |
| M15877 | maltotriose | C01835 | Carbohydrate | Fructose,_mannose,_galactose,_starch,_and_sucrose_metabolism |
| M15910 | maltotetraose | C02052 | Carbohydrate | Fructose,_mannose,_galactose,_starch,_and_sucrose_metabolism |
| M31266 | fructose | C00095 | Carbohydrate | Fructose,_mannose,_galactose,_starch,_and_sucrose_metabolism |
| M577 | fructose | C00095 | Carbohydrate | Fructose,_mannose,_galactose,_starch,_and_sucrose_metabolism |
| M584 | mannose | C00159 | Carbohydrate | Fructose,_mannose,_galactose,_starch,_and_sucrose_metabolism |
| M12021 | fructose-6-phosphate | C05345 | Carbohydrate | Glycolysis,_gluconeogenesis,_pyruvate_metabolism |
| M1414 | 3-phosphoglycerate | C00597 | Carbohydrate | Glycolysis,_gluconeogenesis,_pyruvate_metabolism |
| M15443 | glucuronate | C00191 | Carbohydrate | Glycolysis,_gluconeogenesis,_pyruvate_metabolism |
| M1572 | glycerate | C00258 | Carbohydrate | Glycolysis,_gluconeogenesis,_pyruvate_metabolism |
| M15926 | fructose 1,6-bisphosphate | C05378 | Carbohydrate | Glycolysis,_gluconeogenesis,_pyruvate_metabolism |
| M20488 | glucose | C00293 | Carbohydrate | Glycolysis,_gluconeogenesis,_pyruvate_metabolism |
| M20675 | 1,5-anhydroglucitol | C07326 | Carbohydrate | Glycolysis,_gluconeogenesis,_pyruvate_metabolism |
| M31260 | glucose-6-phosphate | C00668 | Carbohydrate | Glycolysis,_gluconeogenesis,_pyruvate_metabolism |
| M36984 | Isobar: fructose 1,6-diphosphate, glucose 1,6-diphosphate | NA | Carbohydrate | Glycolysis,_gluconeogenesis,_pyruvate_metabolism |
| M527 | lactate | C00186 | Carbohydrate | Glycolysis,_gluconeogenesis,_pyruvate_metabolism |
| M599 | pyruvate | C00022 | Carbohydrate | Glycolysis,_gluconeogenesis,_pyruvate_metabolism |
| M12083 | ribose | C00121 | Carbohydrate | Nucleotide_sugars,_pentose_metabolism |
| M1475 | ribulose 5-phosphate | C00199 | Carbohydrate | Nucleotide_sugars,_pentose_metabolism |
| M15442 | 6-phosphogluconate | C00345 | Carbohydrate | Nucleotide_sugars,_pentose_metabolism |
| M15772 | ribitol | C00474 | Carbohydrate | Nucleotide_sugars,_pentose_metabolism |
| M15835 | xylose | NA | Carbohydrate | Nucleotide_sugars,_pentose_metabolism |
| M15964 | arabitol | C00474 | Carbohydrate | Nucleotide_sugars,_pentose_metabolism |
| M18344 | xylulose | C00310 | Carbohydrate | Nucleotide_sugars,_pentose_metabolism |
| M2763 | UDP-glucuronate | C00167 | Carbohydrate | Nucleotide_sugars,_pentose_metabolism |
| M32344 | UDP-glucose | C00029 | Carbohydrate | Nucleotide_sugars,_pentose_metabolism |
| M32976 | UDP-glucose | C00029 | Carbohydrate | Nucleotide_sugars,_pentose_metabolism |
| M35162 | UDP-N-acetylglucosamine | C00043 | Carbohydrate | Nucleotide_sugars,_pentose_metabolism |
| M35855 | ribulose | C00309 | Carbohydrate | Nucleotide_sugars,_pentose_metabolism |
| M4966 | xylitol | C00379 | Carbohydrate | Nucleotide_sugars,_pentose_metabolism |
| M561 | ribose 5-phosphate | C00117 | Carbohydrate | Nucleotide_sugars,_pentose_metabolism |
| M575 | arabinose | C00181 | Carbohydrate | Nucleotide_sugars,_pentose_metabolism |
| M587 | gluconate | C00257 | Carbohydrate | Nucleotide_sugars,_pentose_metabolism |
| M1640 | ascorbate | C00072 | Cofactors_and_vitamins | Ascorbate_and_aldarate_metabolism |
| M33454 | gulono-1,4-lactone | C01040 | Cofactors_and_vitamins | Ascorbate_and_aldarate_metabolism |
| M32593 | heme* | C00032 | Cofactors_and_vitamins | Hemoglobin_and_porphyrin |
| M1899 | quinolinate | C03722 | Cofactors_and_vitamins | Nicotinate_and_nicotinamide_metabolism |
| M22152 | nicotinamide ribonucleotide | C00455 | Cofactors_and_vitamins | Nicotinate_and_nicotinamide_metabolism |
| M27665 | 1-methylnicotinamide | C02918 | Cofactors_and_vitamins | Nicotinate_and_nicotinamide_metabolism |
| M31475 | nicotinamide adenine dinucleotide reduced | C00004 | Cofactors_and_vitamins | Nicotinate_and_nicotinamide_metabolism |
| M32380 | nicotinamide adenine dinucleotide phosphate | C00005 | Cofactors_and_vitamins | Nicotinate_and_nicotinamide_metabolism |
| M32401 | trigonelline | C01004 | Cofactors_and_vitamins | Nicotinate_and_nicotinamide_metabolism |
| M33013 | nicotinamide riboside | C03150 | Cofactors_and_vitamins | Nicotinate_and_nicotinamide_metabolism |
| M5278 | nicotinamide adenine dinucleotide | C00003 | Cofactors_and_vitamins | Nicotinate_and_nicotinamide_metabolism |
| M558 | adenosine 5′diphosphoribose | C00301 | Cofactors_and_vitamins | Nicotinate_and_nicotinamide_metabolism |
| M594 | nicotinamide | C00153 | Cofactors_and_vitamins | Nicotinate_and_nicotinamide_metabolism |
| M1508 | pantothenate | C00864 | Cofactors_and_vitamins | Pantothenate_and_CoA_metabolism |
| M18289 | 3′-dephosphocoenzyme A | C00882 | Cofactors_and_vitamins | Pantothenate_and_CoA_metabolism |
| M2936 | coenzyme A | C00010 | Cofactors_and_vitamins | Pantothenate_and_CoA_metabolism |
| M1827 | riboflavin | C00255 | Cofactors_and_vitamins | Riboflavin_metabolism |
| M2134 | flavin adenine dinucleotide | C00016 | Cofactors_and_vitamins | Riboflavin_metabolism |
| M5341 | thiamin | C00378 | Cofactors_and_vitamins | Thiamine_metabolism |
| M1561 | alpha-tocopherol | C02477 | Cofactors_and_vitamins | Tocopherol_metabolism |
| M33420 | gamma-tocopherol | C02483 | Cofactors_and_vitamins | Tocopherol_metabolism |
| M31555 | pyridoxate | C00847 | Cofactors_and_vitamins | Vitamin_B6_metabolism |
| M12025 | cis-aconitate | C00417 | Energy | Krebs_cycle |
| M12110 | isocitrate | C00311 | Energy | Krebs_cycle |
| M1303 | malate | C00149 | Energy | Krebs_cycle |
| M1437 | succinate | C00042 | Energy | Krebs_cycle |
| M1564 | citrate | C00158 | Energy | Krebs_cycle |
| M1643 | fumarate | C00122 | Energy | Krebs_cycle |
| M33453 | alpha-ketoglutarate | C00026 | Energy | Krebs_cycle |
| M37058 | succinylcarnitine | NA | Energy | Krebs_cycle |
| M11438 | phosphate | C00009 | Energy | Oxidative_phosphorylation |
| M15488 | acetylphosphate | C00227 | Energy | Oxidative_phosphorylation |
| M2078 | pyrophosphate | C00013 | Energy | Oxidative_phosphorylation |
| M1114 | deoxycholate | C04483 | Lipid | Bile_acid_metabolism |
| M15500 | carnitine | C00487 | Lipid | Carnitine_metabolism |
| M22189 | palmitoylcarnitine | C02990 | Lipid | Carnitine_metabolism |
| M32198 | acetylcarnitine | C02571 | Lipid | Carnitine_metabolism |
| M32328 | hexanoylcarnitine | C01585 | Lipid | Carnitine_metabolism |
| M32654 | 3-dehydrocarnitine | C02636 | Lipid | Carnitine_metabolism |
| M34409 | stearoylcarnitine | NA | Lipid | Carnitine_metabolism |
| M35160 | oleoylcarnitine | NA | Lipid | Carnitine_metabolism |
| M36747 | deoxycarnitine | C01181 | Lipid | Carnitine_metabolism |
| M7746 | prostaglandin E2 | C00584 | Lipid | Eicosanoid |
| M18467 | eicosapentaenoate | C06428 | Lipid | Essential_fatty_acid |
| M19323 | docosahexaenoate | C06429 | Lipid | Essential_fatty_acid |
| M32504 | docosapentaenoate | C16513 | Lipid | Essential_fatty_acid |
| M34035 | linolenate [alpha or gamma (18:3n3 or 6)] | C06427 | Lipid | Essential_fatty_acid |
| M35718 | dihomo-linolenate | C03242 | Lipid | Essential_fatty_acid |
| M37478 | docosapentaenoate | C06429 | Lipid | Essential_fatty_acid |
| M31850 | butyrylglycine | NA | Lipid | Fatty_acid,_beta-oxidation |
| M35436 | hexanoylglycine | NA | Lipid | Fatty_acid,_beta-oxidation |
| M18362 | azelate | C08261 | Lipid | Fatty_acid,_dicarboxylate |
| M31787 | 3-carboxy-4-methyl-5-propyl-2-furanpropanoate | NA | Lipid | Fatty_acid,_dicarboxylate |
| M32398 | sebacate | C08277 | Lipid | Fatty_acid,_dicarboxylate |
| M37253 | 2-hydroxyglutarate | C02630 | Lipid | Fatty_acid,_dicarboxylate |
| M36802 | n-Butyl Oleate | NA | Lipid | Fatty_acid,_ester |
| M17945 | 2-hydroxystearate | C03045 | Lipid | Fatty_acid,_monohydroxy |
| M34585 | 4-hydroxybutyrate | C00989 | Lipid | Fatty_acid,_monohydroxy |
| M35675 | 2_hydroxypalmitate | NA | Lipid | Fatty_acid,_monohydroxy |
| M37752 | 13-HODE 9-HODE | NA | Lipid | Fatty_acid,_monohydroxy |
| M34406 | valerylcarnitine | NA | Lipid | Fatty_acid_metabolism |
| M32412 | butyrylcarnitine | C02862 | Lipid | Fatty_acid_metabolism_(also_BCAA_metabolism) |
| M32452 | propionylcarnitine | C03017 | Lipid | Fatty_acid_metabolism_(also_BCAA_metabolism) |
| M12102 | phosphoethanolamine | C00346 | Lipid | Glycerolipid_metabolism |
| M1497 | ethanolamine | C00189 | Lipid | Glycerolipid_metabolism |
| M15122 | glycerol | C00116 | Lipid | Glycerolipid_metabolism |
| M15365 | glycerol 3-phosphate | C00093 | Lipid | Glycerolipid_metabolism |
| M15506 | choline | C00114 | Lipid | Glycerolipid_metabolism |
| M15990 | glycerophosphoryl choline | C00670 | Lipid | Glycerolipid_metabolism |
| M1600 | phosphoethanolamine | C00346 | Lipid | Glycerolipid_metabolism |
| M34396 | choline phosphate | C00588 | Lipid | Glycerolipid_metabolism |
| M34418 | cytidine 5′-diphosphocholine | C00307 | Lipid | Glycerolipid_metabolism |
| M37455 | glycerophosphoethanolamine | C01233 | Lipid | Glycerolipid_metabolism |
| M1481 | inositol 1-phosphate | C01177 | Lipid | Inositol_metabolism |
| M19934 | myo-inositol | C00137 | Lipid | Inositol_metabolism |
| M32379 | scyllo-inositol | C06153 | Lipid | Inositol_metabolism |
| M542 | 3-hydroxybutyrate | C01089 | Lipid | Ketone_bodies |
| M1105 | linoleate | C01595 | Lipid | Long_chain_fatty_acid |
| M1110 | arachidonate | C00219 | Lipid | Long_chain_fatty_acid |
| M1121 | margarate | NA | Lipid | Long_chain_fatty_acid |
| M1336 | palmitate | C00249 | Lipid | Long_chain_fatty_acid |
| M1356 | nonadecanoate | C16535 | Lipid | Long_chain_fatty_acid |
| M1358 | stearate | C01530 | Lipid | Long_chain_fatty_acid |
| M1359 | oleate | C00712 | Lipid | Long_chain_fatty_acid |
| M1361 | pentadecanoate | C16537 | Lipid | Long_chain_fatty_acid |
| M1365 | myristate | C06424 | Lipid | Long_chain_fatty_acid |
| M17805 | dihomo-linoleate | C16525 | Lipid | Long_chain_fatty_acid |
| M32415 | docosadienoate | C16533 | Lipid | Long_chain_fatty_acid |
| M32417 | docosatrienoate | C16534 | Lipid | Long_chain_fatty_acid |
| M32418 | myristoleate | C08322 | Lipid | Long_chain_fatty_acid |
| M32501 | dihomo-alpha-linolenate | NA | Lipid | Long_chain_fatty_acid |
| M32980 | adrenate | C16527 | Lipid | Long_chain_fatty_acid |
| M33447 | palmitoleate | C08362 | Lipid | Long_chain_fatty_acid |
| M33587 | eicosenoate | NA | Lipid | Long_chain_fatty_acid |
| M33970 | cis-vaccenate | C08367 | Lipid | Long_chain_fatty_acid |
| M33971 | 10-heptadecenoate | NA | Lipid | Long_chain_fatty_acid |
| M33972 | 10-nonadecenoate | NA | Lipid | Long_chain_fatty_acid |
| M35174 | mead acid | NA | Lipid | Long_chain_fatty_acid |
| M19260 | 1-oleoylglycerophosphoserine | NA | Lipid | Lysolipid |
| M19324 | 1-stearoylglycerophosphoinositol | NA | Lipid | Lysolipid |
| M32635 | 1-linoleoylglycerophosphoethanolamine | NA | Lipid | Lysolipid |
| M33871 | 1-eicosadienoylglycerophosphocholine | NA | Lipid | Lysolipid |
| M33955 | 1-palmitoylglycerophosphocholine | C04102 | Lipid | Lysolipid |
| M33960 | 1-oleoylglycerophosphocholine | C03916 | Lipid | Lysolipid |
| M33961 | 1-stearoylglycerophosphocholine | NA | Lipid | Lysolipid |
| M34214 | 1-arachidonoylglycerophosphoinositol | NA | Lipid | Lysolipid |
| M34258 | 2-docosahexaenoylglycerophosphoethanolamine | NA | Lipid | Lysolipid |
| M34416 | 1-stearoylglycerophosphoethanolamine | NA | Lipid | Lysolipid |
| M34419 | 1-linoleoylglycerophosphocholine | C04100 | Lipid | Lysolipid |
| M34656 | 2-arachidonoylglycerophosphoethanolamine | NA | Lipid | Lysolipid |
| M34875 | 2-docosapentaenoylglycerophosphoethanolamine | NA | Lipid | Lysolipid |
| M35186 | 1-arachidonoylglycerophosphoethanolamine | NA | Lipid | Lysolipid |
| M35253 | 2-palmitoylglycerophosphocholine | NA | Lipid | Lysolipid |
| M35254 | 2-oleoylglycerophosphocholine | NA | Lipid | Lysolipid |
| M35256 | 2-arachidonoylglycerophosphocholine | NA | Lipid | Lysolipid |
| M35257 | 2-linoleoylglycerophosphocholine | NA | Lipid | Lysolipid |
| M35305 | 1-palmitoylglycerophosphoinositol | NA | Lipid | Lysolipid |
| M35626 | 1-myristoylglycerophosphocholine | NA | Lipid | Lysolipid |
| M35628 | 1-oleoylglycerophosphoethanolamine | NA | Lipid | Lysolipid |
| M35631 | 1-palmitoylglycerophosphoethanolamine | NA | Lipid | Lysolipid |
| M35687 | 2_oleoylglycerophosphoethanolamine | NA | Lipid | Lysolipid |
| M35688 | 2_palmitoylglycerophosphoethanolamine | NA | Lipid | Lysolipid |
| M36602 | 1-oleoylglycerophosphoinositol | NA | Lipid | Lysolipid |
| M12035 | pelargonate | C01601 | Lipid | Medium_chain_fatty_acid |
| M12067 | undecanoate | NA | Lipid | Medium_chain_fatty_acid |
| M1642 | caprate | C01571 | Lipid | Medium_chain_fatty_acid |
| M1644 | heptanoate | NA | Lipid | Medium_chain_fatty_acid |
| M1645 | laurate | C02679 | Lipid | Medium_chain_fatty_acid |
| M33968 | 5-dodecenoate | NA | Lipid | Medium_chain_fatty_acid |
| M21127 | 1-palmitoylglycerol | NA | Lipid | Monoacylglycerol |
| M21188 | 1-stearoylglycerol | D01947 | Lipid | Monoacylglycerol |
| M27447 | 1-linoleoylglycerol | NA | Lipid | Monoacylglycerol |
| M33419 | 2-palmitoylglycerol | NA | Lipid | Monoacylglycerol |
| M34397 | 1-arachidonylglycerol | C13857 | Lipid | Monoacylglycerol |
| M18790 | acetylcholine | C01996 | Lipid | Neurotransmitter |
| M17747 | sphingosine | C00319 | Lipid | Sphingolipid |
| M19503 | stearoyl sphingomyelin | C00550 | Lipid | Sphingolipid |
| M37506 | palmitoyl sphingomyelin | NA | Lipid | Sphingolipid |
| M32425 | dehydroisoandrosterone sulfate | C04555 | Lipid | Sterol/Steroid |
| M33997 | campesterol | C01789 | Lipid | Sterol/Steroid |
| M35092 | 7-beta-hydroxycholesterol | C03594 | Lipid | Sterol/Steroid |
| M36776 | 7-alpha-hydroxy-3-oxo-4-cholestenoate | C17337 | Lipid | Sterol/Steroid |
| M37202 | 4-androsten-3beta,17beta-diol disulfate 1 | NA | Lipid | Sterol/Steroid |
| M63 | cholesterol | C00187 | Lipid | Sterol/Steroid |
| M37419 | 1-heptadecanoylglycerophosphoethanolamine | NA | No_Super_Pathway | No_Pathway |
| M37070 | methylphosphate | NA | Nucleotide | Purine_and_pyrimidine_metabolism |
| M1123 | inosine | C00294 | Nucleotide | Purine_metabolism,_(hypo)xanthine/inosine_containing |
| M15076 | 2′-deoxyinosine | C05512 | Nucleotide | Purine_metabolism,_(hypo)xanthine/inosine_containing |
| M15136 | xanthosine | C01762 | Nucleotide | Purine_metabolism,_(hypo)xanthine/inosine_containing |
| M3127 | hypoxanthine | C00262 | Nucleotide | Purine_metabolism,_(hypo)xanthine/inosine_containing |
| M3147 | xanthine | C00385 | Nucleotide | Purine_metabolism,_(hypo)xanthine/inosine_containing |
| M15650 | N1-methyladenosine | C02494 | Nucleotide | Purine_metabolism,_adenine_containing |
| M18360 | adenylosuccinate | C03794 | Nucleotide | Purine_metabolism,_adenine_containing |
| M3108 | adenosine 5′-diphosphate | C00008 | Nucleotide | Purine_metabolism,_adenine_containing |
| M32342 | adenosine 5′-monophosphate | C00020 | Nucleotide | Purine_metabolism,_adenine_containing |
| M33449 | adenosine 5′-triphosphate | C00002 | Nucleotide | Purine_metabolism,_adenine_containing |
| M35142 | adenosine 3′-monophosphate | C01367 | Nucleotide | Purine_metabolism,_adenine_containing |
| M36815 | adenosine 2′-monophosphate | C00946 | Nucleotide | Purine_metabolism,_adenine_containing |
| M554 | adenine | C00147 | Nucleotide | Purine_metabolism,_adenine_containing |
| M555 | adenosine | C00212 | Nucleotide | Purine_metabolism,_adenine_containing |
| M1573 | guanosine | C00387 | Nucleotide | Purine_metabolism,_guanine_containing |
| M2849 | guanosine 5′-monophosphate | C00144 | Nucleotide | Purine_metabolism,_guanine_containing |
| M31609 | N1-methylguanosine | NA | Nucleotide | Purine_metabolism,_guanine_containing |
| M32352 | guanine | C00242 | Nucleotide | Purine_metabolism,_guanine_containing |
| M418 | guanine | C00242 | Nucleotide | Purine_metabolism,_guanine_containing |
| M1107 | allantoin | C02350 | Nucleotide | Purine_metabolism,_urate_metabolism |
| M1604 | urate | C00366 | Nucleotide | Purine_metabolism,_urate_metabolism |
| M37465 | cytosine 2′ 3′ cyclic monophosphate | NA | Nucleotide | Pyrimidine metabolism (cytidine-containing) |
| M2372 | cytidine 5′-monophosphate | C00055 | Nucleotide | Pyrimidine_metabolism,_cytidine_containing |
| M2959 | cytidine-3′-monophosphate | C05822 | Nucleotide | Pyrimidine_metabolism,_cytidine_containing |
| M514 | cytidine | C00475 | Nucleotide | Pyrimidine_metabolism,_cytidine_containing |
| M1505 | orotate | C00295 | Nucleotide | Pyrimidine_metabolism,_orotate_containing |
| M1566 | 3-aminoisobutyrate | C05145 | Nucleotide | Pyrimidine_metabolism,_thymine_containing;_Valine,_leucine_and_isoleucine_metabolism/ |
| M1559 | 5,6-dihydrouracil | C00429 | Nucleotide | Pyrimidine_metabolism,_uracil_containing |
| M2856 | uridine 5′-monophosphate | C00105 | Nucleotide | Pyrimidine_metabolism,_uracil_containing |
| M33442 | pseudouridine | C02067 | Nucleotide | Pyrimidine_metabolism,_uracil_containing |
| M37137 | uridine-2′,3′-cyclicmonophosphate | C02355 | Nucleotide | Pyrimidine_metabolism,_uracil_containing |
| M5345 | uridine 5′-diphosphate | C00015 | Nucleotide | Pyrimidine_metabolism,_uracil_containing |
| M605 | uracil | C00106 | Nucleotide | Pyrimidine_metabolism,_uracil_containing |
| M606 | uridine | C00299 | Nucleotide | Pyrimidine_metabolism,_uracil_containing |
| M22171 | glycylproline | NA | Peptide | Dipeptide |
| M22175 | aspartylphenylalanine | NA | Peptide | Dipeptide |
| M31530 | threonylphenylalanine | NA | Peptide | Dipeptide |
| M32393 | glutamylvaline | NA | Peptide | Dipeptide |
| M32394 | pyroglutamylvaline | NA | Peptide | Dipeptide |
| M33958 | glycyltyrosine | NA | Peptide | Dipeptide |
| M34398 | glycylleucine | C02155 | Peptide | Dipeptide |
| M35637 | cysteinylglycine | C01419 | Peptide | Dipeptide |
| M36659 | glycylisoleucine | NA | Peptide | Dipeptide |
| M36756 | leucylleucine | C11332 | Peptide | Dipeptide |
| M36761 | isoleucylisoleucine | NA | Peptide | Dipeptide |
| M37093 | alanylleucine | NA | Peptide | Dipeptide |
| M37098 | alanyltyrosine | NA | Peptide | Dipeptide |
| M15747 | anserine | C01262 | Peptide | Dipeptide_derivative |
| M1633 | homocarnosine | C00884 | Peptide | Dipeptide_derivative |
| M1768 | carnosine | C00386 | Peptide | Dipeptide_derivative |
| M18369 | gamma-glutamylleucine | NA | Peptide | gamma-glutamyl |
| M2730 | gamma-glutamylglutamine | NA | Peptide | gamma-glutamyl |
| M36738 | gamma-glutamylglutamate | NA | Peptide | gamma-glutamyl |
| M37063 | gamma-glutamylalanine | NA | Peptide | gamma-glutamyl |
| M37539 | gamma-glutamylmethionine | NA | Peptide | gamma-glutamyl |
| M34456 | gamma-glutamylisoleucine | NA | Peptide | g-glutamyl |
| M15753 | hippurate | C01586 | Xenobiotics | Benzoate_metabolism |
| M18281 | 2-hydroxyhippurate | C07588 | Xenobiotics | Benzoate_metabolism |
| M35320 | catechol sulfate | C00090 | Xenobiotics | Benzoate_metabolism |
| M36098 | 4-vinylphenol sulfate | C05627 | Xenobiotics | Benzoate_metabolism |
| M36099 | 4-ethylphenylsulfate | NA | Xenobiotics | Benzoate_metabolism |
| M1554 | 2-ethylhexanoate | NA | Xenobiotics | Chemical |
| M20714 | methyl-alpha-glucopyranoside | C03619 | Xenobiotics | Chemical |
| M27728 | glycerol 2-phosphate | C02979 | Xenobiotics | Chemical |
| M27743 | triethyleneglycol | NA | Xenobiotics | Chemical |
| M12032 | 4-acetamidophenol | C06804 | Xenobiotics | Drug |
| M33080 | N-ethylglycinexylidide | C16561 | Xenobiotics | Drug |
| M33173 | 2-hydroxyacetaminophen sulfate | NA | Xenobiotics | Drug |
| M33178 | 2-methoxyacetaminophen sulfate | NA | Xenobiotics | Drug |
| M33423 | p-acetamidophenylglucuronide | NA | Xenobiotics | Drug |
| M34346 | desmethylnaproxen sulfate | NA | Xenobiotics | Drug |
| M34365 | 3-(cystein-S-yl)acetaminophen | NA | Xenobiotics | Drug |
| M35661 | lidocaine | D00358 | Xenobiotics | Drug |
| M37468 | penicillin G | C05551 | Xenobiotics | Drug |
| M37475 | 4-acetaminophen sulfate | C06804 | Xenobiotics | Drug |
| M38637 | cinnamoylglycine | NA | Xenobiotics | Food component (plant) |
| M18335 | quinate | C00296 | Xenobiotics | Food_component/Plant |
| M32448 | genistein | C06563 | Xenobiotics | Food_component/Plant |
| M32453 | daidzein | C10208 | Xenobiotics | Food_component/Plant |
| M33935 | piperine | C03882 | Xenobiotics | Food_component/Plant |
| M37459 | ergothioneine | C05570 | Xenobiotics | Food_component/Plant |
| M20699 | erythritol | C00503 | Xenobiotics | Sugar,_sugar_substitute,_starch |
| M18254 | paraxanthine | C13747 | Xenobiotics | Xanthine_metabolism |
| M18392 | theobromine | C07480 | Xenobiotics | Xanthine_metabolism |
| M34400 | 1,7-dimethylurate | C16356 | Xenobiotics | Xanthine_metabolism |
| M569 | caffeine | C07481 | Xenobiotics | Xanthine_metabolism |

TABLE 2
Metabolite concentration fold changes and p-values for RWPE-AKT1 cells, MPAKT mice and phosphoAKT1-high/MYC-
low tumors compared to RWPE-MYC cells, Lo-MYC mice and MYC-high/phosphoAKT1-low tumors, respectively.

Table 2: RWPE cells

| | | | | | Fold Change |
|---|---|---|---|---|---|
| | KEGG | | | | (RWPE-AKT1/ |
| Metabolite | ID | Statistic | Pvalue | BH | RWPE-MYC) |
| fructose_1,6-bisphosphate | C05378 | 119.8676864 | 0.009998 | 0.020353072 | 4.738624407 |
| glucose | C00267 | 20.65226182 | 0.009998 | 0.020353072 | 51.51377553 |
| kynurenine | C00328 | 15.70155617 | 0.009998 | 0.020353072 | 3.045622149 |
| hypoxanthine | C00262 | 13.70619099 | 0.009998 | 0.020353072 | 2.286526654 |
| 1-palmitoylglycerophosphocholine | C04102 | 10.4032463 | 0.009998 | 0.020353072 | 5.157499278 |
| ribulose_5-phosphate | C00117.2 | 9.265638432 | 0.009998 | 0.020353072 | 3.76062704 |
| arachidonate | C00219 | 9.18187886 | 0.009998 | 0.020353072 | 2.097490562 |
| docosahexaenoate | C06429 | 9.07763373 | 0.009998 | 0.020353072 | 2.48420095 |
| ribose_5-phosphate | C00117 | 8.418309746 | 0.009998 | 0.020353072 | 9.618227338 |
| N-acetylneuraminate | C00270 | 8.277850689 | 0.009998 | 0.020353072 | 2.462617276 |
| palmitoylcarnitine | C02990 | 7.163347714 | 0.009998 | 0.020353072 | 4.155427482 |
| docosapentaenoate | C16513 | 6.356127711 | 0.009998 | 0.020353072 | 2.024159333 |
| lactate | C00186 | 6.086634561 | 0.009998 | 0.020353072 | 1.979031832 |
| threonine | C00188 | 5.424535734 | 0.009998 | 0.020353072 | 1.20625138 |
| sphingosine | C00319 | 4.927267217 | 0.009998 | 0.020353072 | 3.942420982 |
| malate | C00149 | 4.84868646 | 0.009998 | 0.020353072 | 1.180212973 |
| putrescine | C00134 | 4.363517765 | 0.009998 | 0.020353072 | 1.716300482 |
| carnitine | C00487 | 4.149148079 | 0.016996601 | 0.032840889 | 1.181253854 |
| serine | C00065 | 4.145286144 | 0.009998 | 0.020353072 | 1.286416228 |
| glutamine | C00064 | 4.145166486 | 0.009998 | 0.020353072 | 1.45086936 |
| tryptophan | C00078 | 4.120933202 | 0.009998 | 0.020353072 | 1.207529259 |
| isoleucine | C00407 | 4.01686246 | 0.018196361 | 0.033457825 | 1.291948938 |
| histidine | C00135 | 3.745126323 | 0.009998 | 0.020353072 | 1.448776697 |
| leucine | C00123 | 3.59956152 | 0.009998 | 0.020353072 | 1.325546255 |
| UDP-glucuronate | C00167 | 3.543974822 | 0.016196761 | 0.032393521 | 1.33853376 |
| phenylalanine | C00079 | 3.404997548 | 0.009998 | 0.020353072 | 1.248853872 |
| guanine | C00242 | 3.315805992 | 0.009998 | 0.020353072 | 2.620464264 |
| tyrosine | C00082 | 3.291334315 | 0.009998 | 0.020353072 | 1.289289976 |
| proline | C00148 | 3.26925609 | 0.009998 | 0.020353072 | 1.594939743 |
| oleate | C00712 | 3.260383573 | 0.031793641 | 0.050973139 | 1.191404393 |
| stearate | C01530 | 3.037917062 | 0.028194361 | 0.046581988 | 1.140029894 |
| asparagine | C00152 | 2.969467579 | 0.018196361 | 0.033457825 | 1.270943015 |
| uracil | C00106 | 2.962293391 | 0.025394921 | 0.043863954 | 1.32443449 |
| nicotinamide_adenine_dinucleotide_reduced | C00004 | 2.84879095 | 0.032193561 | 0.050973139 | 1.380002307 |
| 1-oleoylglycerophosphocholine | C03916 | 2.674874262 | 0.009998 | 0.020353072 | 2.483788951 |
| ornithine | C00077 | 2.561400158 | 0.060387922 | 0.091789642 | 1.253497526 |
| gulono-1,4-lactone | C01040 | 2.218229087 | 0.047990402 | 0.07393116 | 1.552385728 |
| valine | C00183 | 2.04152656 | 0.076984603 | 0.112515958 | 1.191354427 |
| uridine | C00299 | 1.623228155 | 0.134573085 | 0.184835322 | 1.33283102 |
| inosine | C00294 | 1.605454242 | 0.155968806 | 0.206749348 | 1.340389058 |
| lysine | C00047 | 1.584268139 | 0.141171766 | 0.19094534 | 1.151833443 |
| choline | C00114 | 1.474667131 | 0.203759248 | 0.263960844 | 1.345176677 |
| adenosine_5′-triphosphate | C00002 | 1.429848319 | 0.215156969 | 0.275594319 | 1.257939688 |
| acetylcarnitine | C02571 | 1.198386024 | 0.24715057 | 0.306251793 | 1.205609184 |
| eicosapentaenoate | C06428 | 1.00058253 | 0.322135573 | 0.394875864 | 1.294857331 |
| 3-phosphoglycerate | C00597 | 0.902580834 | 0.398520296 | 0.468364059 | 1.306676106 |
| propionylcarnitine | C03017 | 0.839896929 | 0.396920616 | 0.468364059 | 1.091033094 |
| beta-alanine | C00099 | 0.596360195 | 0.564487103 | 0.625214763 | 1.100163038 |
| methionine | C00073 | 0.585286137 | 0.638072386 | 0.6994255 | 1.048323339 |
| betaine | C00719 | 0.487208252 | 0.659468106 | 0.715993944 | 1.085759788 |
| alanine | C00041 | 0.458235877 | 0.797640472 | 0.82664558 | 1.014615243 |
| glutathione,_oxidized | C00127 | 0.456820572 | 0.698860228 | 0.737685796 | 1.026015667 |
| adrenate | C16527 | 0.119820035 | 0.99320136 | 0.99320136 | 1.081585609 |
| UDP-N-acetylglucosamine | C00043 | 0.097138409 | 0.912417516 | 0.928710686 | 1.020584246 |
| glycine | C00037 | 0.082512239 | 0.922415517 | 0.930578486 | 1.004706923 |
| nicotinamide | C00153 | −0.112901051 | 0.896620676 | 0.920853667 | 1.02609055 |
| cholesterol | C00187 | −0.319104758 | 0.769646071 | 0.804950936 | 1.020336263 |
| glutamate | C00025 | −0.374926118 | 0.685462907 | 0.737195957 | 1.012534599 |
| urea | C00086 | −0.427064095 | 0.696860628 | 0.737685796 | 1.082956245 |
| gamma-aminobutyrate | C00334 | −0.590636165 | 0.564887023 | 0.625214763 | 1.078318168 |
| 5-oxoproline | C01879 | −0.651258364 | 0.518696261 | 0.597286603 | 1.101709722 |
| palmitate | C00249 | −0.687287197 | 0.5034993 | 0.585703268 | 1.065072979 |
| UDP-glucose | C00029 | −0.829664305 | 0.548490302 | 0.619088064 | 1.25586995 |
| S-adenosylhomocysteine | C00021 | −0.942596354 | 0.363727255 | 0.436472705 | 1.060712256 |
| ascorbate | C00072 | −0.951130088 | 0.530693861 | 0.604991002 | 1.196504701 |
| pentadecanoate | C16537 | −0.977207694 | 0.350729854 | 0.425353227 | 1.560279788 |
| guanosine_5′-_monophosphate | C00144 | −1.291713384 | 0.226154769 | 0.283314766 | 1.298414486 |
| caprate | C01571 | −1.322571824 | 0.193561288 | 0.253632032 | 1.12199237 |
| 5-methylthioadenosine | C00170 | −1.381370394 | 0.220755849 | 0.279624075 | 1.106583491 |
| adenosine_5′-diphosphate | C00008 | −1.505298233 | 0.00959808 | 0.020353072 | 1.596442366 |
| fructose-6-phosphate | C05345 | −1.647814474 | 0.142371526 | 0.19094534 | 1.614290762 |
| cytidine_5′-diphosphocholine | C00307 | −1.648425787 | 0.101179764 | 0.142401149 | 1.118772265 |
| guanosine | C00387 | −1.793286389 | 0.098780244 | 0.140761848 | 1.462171557 |
| inositol_1-phosphate | C01177 | −1.795127438 | 0.119176165 | 0.165683936 | 1.527744384 |
| adenine | C00147 | −1.799549967 | 0.073185363 | 0.108352356 | 1.246252244 |
| pelargonate | C01601 | −1.839428678 | 0.087382523 | 0.1260963 | 1.24618128 |
| hypotaurine | C00519 | −2.072758483 | 0.064187163 | 0.096280744 | 1.42070142 |
| cysteine | C00097 | −2.222496709 | 0.031993601 | 0.050973139 | 2.267677642 |
| adenylosuccinate | C03794 | −2.287317591 | 2.00E−04 | 9.12E−04 | 10.83302465 |
| linoleate | C01595 | −2.345538274 | 0.045990802 | 0.071821252 | 1.19157127 |
| arginine | C00062 | −2.355786576 | 2.00E−04 | 9.12E−04 | 1.498777516 |
| glycerol_3-phosphate | C00093 | −2.36261644 | 0.026194761 | 0.043914746 | 1.512845547 |
| scyllo-inositol | C06153 | −2.444498861 | 0.017796441 | 0.033457825 | 1.570691312 |
| palmitoleate | C08362 | −2.469099284 | 0.023195361 | 0.041972558 | 1.348678628 |
| pyrophosphate | C00013 | −2.499282383 | 2.00E−04 | 9.12E−04 | 22.19112918 |
| spermidine | C00315 | −2.547175419 | 0.024195161 | 0.04309763 | 2.930265588 |
| creatine | C00300 | −2.807552448 | 0.025194961 | 0.043863954 | 1.511098738 |
| glutathione,_reduced | C00051 | −2.833137036 | 0.00879824 | 0.020353072 | 1.274109649 |
| laurate | C02679 | −2.94364984 | 2.00E−04 | 9.12E−04 | 1.467115317 |
| acetylphosphate | C00227 | −3.048003937 | 2.00E−04 | 9.12E−04 | 1.224222457 |
| adenosine | C00212 | −3.176824097 | 0.025994801 | 0.043914746 | 1.301957807 |
| nicotinamide_adenine_dinucleotide_phosphate | C00005 | −3.261185332 | 0.016996601 | 0.032840889 | 1.631472907 |
| myristoleate | C08322 | −3.297885963 | 2.00E−04 | 9.12E−04 | 1.709976347 |
| glucose-6-phosphate | C00668 | −3.660174305 | 2.00E−04 | 9.12E−04 | 2.345491734 |
| citrate | C00158 | −3.834436092 | 0.00859828 | 0.020353072 | 1.236763118 |
| cytidine_5′-monophosphate | C00055 | −4.096483485 | 2.00E−04 | 9.12E−04 | 1.811603131 |
| myristate | C06424 | −4.20707113 | 0.00679864 | 0.020353072 | 1.489863819 |
| myo-inositol | C00137 | −4.259788648 | 2.00E−04 | 9.12E−04 | 1.370642583 |
| fumarate | C00122 | −4.268976999 | 2.00E−04 | 9.12E−04 | 1.510804551 |
| uridine_5′-monophosphate | C00105 | −4.310103285 | 2.00E−04 | 9.12E−04 | 1.922261646 |
| spermine | C00750 | −4.526787877 | 2.00E−04 | 9.12E−04 | 3.934229574 |
| glycerophosphorylcholine | C00670 | −4.609315684 | 2.00E−04 | 9.12E−04 | 7.148421913 |
| 1-methylnicotinamide | C02918 | −5.093201852 | 2.00E−04 | 9.12E−04 | 1.259641237 |
| butyrylcarnitine | C02862 | −5.435624344 | 2.00E−04 | 9.12E−04 | 1.544844116 |
| fructose | C00095 | −6.698792894 | 2.00E−04 | 9.12E−04 | 2.160039345 |
| choline_phosphate | C00588 | −8.453823521 | 2.00E−04 | 9.12E−04 | 1.810762669 |
| adenosine_5′-monophosphate | C00020 | −8.969613192 | 2.00E−04 | 9.12E−04 | 2.021279539 |
| S-lactoylglutathione | C03451 | −10.3263094 | 2.00E−04 | 9.12E−04 | 3.238772345 |
| aspartate | C00049 | −10.42113385 | 2.00E−04 | 9.12E−04 | 1.672765754 |
| pantothenate | C00864 | −10.55863989 | 2.00E−04 | 9.12E−04 | 2.38346229 |
| nicotinamide_adenine_dinucleotide | C00003 | −10.70673596 | 2.00E−04 | 9.12E−04 | 2.061232441 |
| phosphate | C00009 | −10.87211685 | 2.00E−04 | 9.12E−04 | 1.939572376 |
| glycerol | C00116 | −11.18675245 | 2.00E−04 | 9.12E−04 | 1.612824216 |
| flavin_adenine_dinucleotide | C00016 | −15.61444522 | 2.00E−04 | 9.12E−04 | 2.813638126 |

Table 2: Mice

| | | | | | Fold Change |
|---|---|---|---|---|---|
| | KEGG | | | | (MPAKT/ |
| Metabolite | ID | Statistic | Pvalue | BH | Lo-MYC) |
| cholesterol | C00187 | 5.731030747 | 0.00219956 | 0.014957009 | 1.314480145 |
| orotate | C00295 | 4.846016945 | 0.00219956 | 0.014957009 | 5.324861974 |
| isoleucine | C00407 | 4.802230236 | 0.00219956 | 0.014957009 | 1.78958409 |
| acetylcarnitine | C02571 | 4.38451587 | 0.00219956 | 0.014957009 | 1.702913689 |
| valine | C00183 | 4.070684752 | 0.00379924 | 0.022465072 | 1.381314289 |
| propionylcarnitine | C03017 | 4.024578503 | 0.00419916 | 0.022843431 | 1.772345283 |
| cytidine_5′-monophosphate | C00055 | 3.928335838 | 0.00219956 | 0.014957009 | 1.662146089 |
| thiamin | C00378 | 3.454652887 | 0.00779844 | 0.030216179 | 1.598836673 |
| malate | C00149 | 3.222867661 | 0.0079984 | 0.030216179 | 1.426765535 |
| lactate | C00186 | 3.172803844 | 0.0069986 | 0.029744051 | 1.803881231 |
| glycine | C00037 | 3.153068661 | 0.018796241 | 0.058097471 | 1.31995762 |
| serine | C00065 | 3.057757208 | 0.016196761 | 0.053094143 | 1.552959004 |
| riboflavin | C00255 | 3.019909796 | 0.014397121 | 0.049630074 | 1.64953552 |
| leucine | C00123 | 2.931057916 | 0.00919816 | 0.033809454 | 1.261816088 |
| scyllo-inositol | C06153 | 2.792377804 | 0.00219956 | 0.014957009 | 3.705486601 |
| mannose | C00159 | 2.752696427 | 0.00219956 | 0.014957009 | 1.959596598 |
| citrate | C00158 | 2.734987498 | 0.030993801 | 0.08781577 | 1.527179249 |
| tryptophan | C00078 | 2.583459194 | 0.00659868 | 0.028949049 | 1.571086987 |
| fructose-6-phosphate | C05345 | 2.580081431 | 0.026594681 | 0.077533429 | 2.491828548 |
| sorbitol | C00794 | 2.443734936 | 0.01159768 | 0.041507488 | 8.880967365 |
| butyrylcarnitine | C02862 | 2.386996272 | 0.026394721 | 0.077533429 | 2.60845214 |
| choline | C00114 | 2.268940153 | 0.068186363 | 0.165595452 | 1.257780595 |
| uridine-2′,3′-cyclic_monophosphate | C02355 | 2.172778942 | 0.0079984 | 0.030216179 | 3.159365678 |
| ascorbate | C00072 | 2.146212519 | 0.048790242 | 0.127605248 | 7.139154413 |
| ribulose_5-phosphate | C00199 | 2.132110125 | 0.034593081 | 0.094093181 | 2.065713503 |
| aspartate | C00049 | 2.086957772 | 0.014597081 | 0.049630074 | 1.69706794 |
| phenylalanine | C00079 | 2.02154555 | 0.054189162 | 0.136476408 | 1.319360097 |
| spermidine | C00315 | 1.885729393 | 0.097180564 | 0.207358528 | 1.869906627 |
| prostaglandin.E2 | C00584 | 1.861764058 | 0.105978804 | 0.215121155 | 3.173288966 |
| glucose-6-phosphate | C00668 | 1.838232381 | 0.091381724 | 0.203736302 | 1.818559261 |
| glycerol | C00116 | 1.744469954 | 0.095380924 | 0.207358528 | 1.378443437 |
| N-acetylglucosamine | C03878 | 1.744364762 | 0.111377724 | 0.216066376 | 5.212405739 |
| adenosine_2′-monophosphate | C00946 | 1.730163833 | 0.185562887 | 0.286779008 | 2.223593936 |
| fructose | C00095 | 1.708754 | 0.00219956 | 0.014957009 | 2.546501911 |
| lysine | C00047 | 1.689904121 | 0.115976805 | 0.216066376 | 1.800193662 |
| glycerol_2-phosphate | C02979 | 1.674246236 | 0.072785443 | 0.170669314 | 1.795693849 |
| tyrosine | C00082 | 1.650959636 | 0.113977205 | 0.216066376 | 1.166769141 |
| mannose-6-phosphate | C00275 | 1.607439929 | 0.132373525 | 0.23687894 | 1.371543598 |
| threonine | C00188 | 1.588042323 | 0.152369526 | 0.254368638 | 1.326419463 |
| ergothioneine | C05570 | 1.566794854 | 0.146370726 | 0.250529894 | 2.047100977 |
| hypotaurine | C00519 | 1.563831201 | 0.153369326 | 0.254368638 | 1.663143775 |
| phenylacetylglycine | C05598 | 1.526261401 | 0.211757648 | 0.299140172 | 2.122666545 |
| phenol_sulfate | C02180 | 1.458425372 | 0.184163167 | 0.286779008 | 2.08333105 |
| hypoxanthine | C00262 | 1.403555145 | 0.184763047 | 0.286779008 | 1.187168862 |
| cis-vaccenate | C08367 | 1.388921857 | 0.24015197 | 0.329905736 | 1.602106655 |
| adenosine_5′-monophosphate | C00301 | 1.376700664 | 0.206958608 | 0.299140172 | 1.737664047 |
| ribose_5-phosphate | C00117 | 1.373386856 | 0.201959608 | 0.299140172 | 1.702070354 |
| glycerol_3-phosphate | C00093 | 1.341635345 | 0.204359128 | 0.299140172 | 1.315510733 |
| creatine | C00300 | 1.290483341 | 0.230153969 | 0.319397345 | 1.179965058 |
| methionine | C00073 | 1.227369038 | 0.25634873 | 0.348634273 | 1.225165514 |
| cystine | C00491 | 1.11255097 | 0.268346331 | 0.361337633 | 1.656942531 |
| erythritol | C00503 | 1.109359211 | 0.367326535 | 0.471286875 | 2.612403046 |
| ribose | C00121 | 0.966345111 | 0.357128574 | 0.465415488 | 1.283462636 |
| isocitrate | C00311 | 0.942410074 | 0.359328134 | 0.465415488 | 1.220029866 |
| carnitine | C00487 | 0.920360002 | 0.403719256 | 0.508387211 | 1.067957102 |
| glucuronate | C00191 | 0.896194973 | 0.579084183 | 0.673123495 | 1.21671571 |
| cis-aconitate | C00417 | 0.678902233 | 0.49030194 | 0.600730304 | 1.092276423 |
| spermine | C00750 | 0.650288357 | 0.536692661 | 0.651698232 | 1.157754191 |
| adenosine_5′diphosphoribose | C00020 | 0.612073031 | 0.554089182 | 0.661018673 | 1.074048371 |
| proline | C00148 | 0.536285082 | 0.571685663 | 0.670252156 | 1.08788306 |
| 7-beta-hydroxycholesterol | C03594 | 0.511913231 | 0.657068586 | 0.750935527 | 1.13242841 |
| oleate | C00712 | 0.495364144 | 0.903619276 | 0.945789468 | 1.24564639 |
| guanine | C00242 | 0.306116255 | 0.911017796 | 0.945789468 | 1.101595185 |
| N1-methyladenosine | C02494 | 0.293397754 | 0.788642272 | 0.875090023 | 1.058292571 |
| S-adenosylhomocysteine | C00021 | 0.287354295 | 0.785042991 | 0.875090023 | 1.067193013 |
| 2-hydroxystearate | C03045 | 0.20387351 | 0.826234753 | 0.903294541 | 1.053769973 |
| arabitol | C00474 | 0.166523847 | 0.908218356 | 0.945789468 | 1.046380428 |
| ethanolamine | C00189 | 0.160019445 | 0.879024195 | 0.941317248 | 1.033889323 |
| inositol_1-phosphate | C01177 | 0.126909671 | 0.902619476 | 0.945789468 | 1.032424174 |
| beta-alanine | C00099 | 0.029358416 | 0.954609078 | 0.976141614 | 1.00604818 |
| urea | C00086 | −0.011263049 | 0.968006399 | 0.982454255 | 1.003877952 |
| glutamine | C00064 | −0.040947438 | 0.985602879 | 0.992903641 | 1.007969293 |
| fucose | C00382 | −0.079293687 | 0.99580084 | 0.99580084 | 1.021033485 |
| stearate | C01530 | −0.120565217 | 0.940611878 | 0.969115268 | 1.027200106 |
| N-acetylneuraminate | C00270 | −0.241637282 | 0.839432114 | 0.90605371 | 1.080760497 |
| glycerophosphorylcholine | C00670 | −0.26074411 | 0.791441712 | 0.875090023 | 1.031687002 |
| alanine | C00041 | −0.339449007 | 0.74605079 | 0.845524228 | 1.072507219 |
| daidzein | C10208 | −0.396828559 | 0.830233953 | 0.903294541 | 1.132101137 |
| phosphoethanolamine | C00346 | −0.529127619 | 0.616476705 | 0.710515524 | 1.137601266 |
| guanosine | C00387 | −0.612164197 | 0.569286143 | 0.670252156 | 1.137600738 |
| creatinine | C00791 | −0.612569424 | 0.543291342 | 0.653872765 | 1.117408011 |
| cytidine | C00475 | −0.752474325 | 0.483103379 | 0.597291451 | 1.038949728 |
| hippurate | C01586 | −0.963850443 | 0.411517696 | 0.513453273 | 1.812097642 |
| dimethylarginine | C03626 | −1.010556019 | 0.330333933 | 0.436169077 | 1.226991293 |
| palmitoleate | C08362 | −1.027651832 | 0.373725255 | 0.475015277 | 1.48008998 |
| allantoin | C02350 | −1.091601809 | 0.322535493 | 0.430047324 | 1.22909512 |
| 1-oleoylglycerophosphocholine | C03916 | −1.235651207 | 0.144171166 | 0.250529894 | 2.299674594 |
| 1-palmitoylglycerophosphocholine | C04102 | −1.313110736 | 0.182963407 | 0.286779008 | 2.398375439 |
| N-acetylglutamine | C02716 | −1.32387446 | 0.209958008 | 0.299140172 | 1.344494727 |
| inosine | C00294 | −1.328685132 | 0.213357329 | 0.299140172 | 1.049579003 |
| nonadecanoate | C16535 | −1.356220614 | 0.204559088 | 0.299140172 | 1.242624843 |
| uridine | C00299 | −1.386031066 | 0.204759048 | 0.299140172 | 1.208592686 |
| glycerate | C00258 | −1.394835645 | 0.165566887 | 0.27129032 | 1.56500217 |
| urocanate | C00785 | −1.414697383 | 0.196760648 | 0.299140172 | 1.066816486 |
| stachydrine | C10172 | −1.423477496 | 0.181763647 | 0.286779008 | 1.076436018 |
| arabinose | C00181 | −1.631168606 | 0.147370526 | 0.250529894 | 1.08338153 |
| linolenate_[alpha_or_gamma;_(18:3n3_or_6)] | C06427 | −1.636346218 | 0.138372326 | 0.244397874 | 2.095455054 |
| genistein | C06563 | −1.642382231 | 0.125774845 | 0.228071719 | 1.133305744 |
| trigonelline | C01004 | −1.647807362 | 0.112977405 | 0.216066376 | 1.478233048 |
| erythronate | C01620 | −1.727643931 | 0.115576885 | 0.216066376 | 1.092209671 |
| xylitol | C00379 | −1.743195697 | 0.112777445 | 0.216066376 | 1.091316003 |
| palmitate | C00249 | −1.746051908 | 0.124575085 | 0.228071719 | 1.40183288 |
| campesterol | C01789 | −1.806224883 | 0.103979204 | 0.214260178 | 1.854858177 |
| 4-guanidinobutanoate | C01035 | −1.835399006 | 0.069986003 | 0.166984147 | 1.794083739 |
| 1-methylimidazoleacetate | C05828 | −1.861896377 | 0.102179564 | 0.213791088 | 1.094316851 |
| choline_phosphate | C00588 | −1.868693128 | 0.097580484 | 0.207358528 | 1.360639257 |
| cystathionine | C02291 | −1.940376865 | 0.075984803 | 0.174045191 | 2.476129175 |
| 3-ureidopropionate | C02642 | −1.994480853 | 0.076784643 | 0.174045191 | 1.102582726 |
| adenosine_3′-monophosphate | C01367 | −2.008881945 | 0.067186563 | 0.165595452 | 1.516930206 |
| cysteine | C00097 | −2.187203269 | 0.045590882 | 0.121575685 | 1.946237595 |
| uridine_5′-monophosphate | C00105 | −2.196658136 | 0.00759848 | 0.030216179 | 3.259640455 |
| 5-oxoproline | C01879 | −2.226686297 | 0.017396521 | 0.055021554 | 2.154245824 |
| alpha-tocopherol | C02477 | −2.23824325 | 0.050589882 | 0.129815546 | 1.111658614 |
| adenine | C00147 | −2.457363752 | 0.032193561 | 0.089353558 | 1.87737174 |
| pantothenate | C00864 | −2.554952589 | 0.016396721 | 0.053094143 | 2.761840905 |
| docosahexaenoate | C06429 | −2.682822525 | 0.00019996 | 0.002472233 | 2.150603511 |
| docosapentaenoate | C16513 | −2.718069448 | 0.0029994 | 0.018541746 | 1.835848861 |
| pyridoxate | C00847 | −2.738352083 | 0.026794641 | 0.077533429 | 1.140050442 |
| cytidine_5′-diphosphocholine | C00307 | −3.093460689 | 0.00659868 | 0.028949049 | 1.687784683 |
| arginine | C00062 | −3.178293058 | 0.00639872 | 0.028949049 | 1.197039482 |
| linoleate | C01595 | −3.341037454 | 0.00479904 | 0.024172943 | 2.648550608 |
| 5-methylthioadenosine | C00170 | −3.672915952 | 0.00559888 | 0.027194561 | 1.77421305 |
| 3-dehydrocarnitine | C02636 | −3.812488098 | 0.00439912 | 0.023010782 | 3.030002448 |
| xanthine | C00385 | −3.974250304 | 0.00019996 | 0.002472233 | 1.416735201 |
| glutamate | C00025 | −4.027289964 | 0.00419916 | 0.022843431 | 1.68866346 |
| phosphate | C00009 | −4.356812631 | 0.00259948 | 0.016834728 | 1.351048477 |
| arachidonate | C00219 | −4.527712505 | 0.00019996 | 0.002472233 | 2.05447056 |
| betaine | C00719 | −4.787930679 | 0.00019996 | 0.002472233 | 2.132690945 |
| nicotinamide | C00153 | −4.833362163 | 0.00019996 | 0.002472233 | 1.242336465 |
| taurine | C00245 | −4.890479424 | 0.00219956 | 0.014957009 | 1.3311185 |
| adenosine | C00212 | −5.526740727 | 0.00019996 | 0.002472233 | 2.130704285 |
| pseudouridine | C02067 | −5.635590595 | 0.00019996 | 0.002472233 | 2.21339505 |
| UDP-glucose | C00029 | −5.738020226 | 0.00019996 | 0.002472233 | 2.727880622 |
| cytidine-3′-monophosphate | C05822 | −5.842264043 | 0.00019996 | 0.002472233 | 3.0266933 |
| dihomo-linolenate | C03242 | −12.06944017 | 0.00019996 | 0.002472233 | 4.764943624 |
| sarcosine | C00213 | −25.32566958 | 0.00019996 | 0.002472233 | 13.98934706 |

Table 2: Human tumors

| | | | | | Fold Change |
|---|---|---|---|---|---|
| | KEGG | | | | (PhosphoAKT1- |
| Metabolite | ID | Statistic | Pvalue | BH | high/MYC-high) |
| fructose-6-phosphate | C05345 | 3.81110406 | 0.00019996 | 0.045590882 | 3.631619045 |
| uridine | C00299 | 3.5590535 | 0.00119976 | 0.078155797 | 1.296349317 |
| leucylleucine | C11332 | 3.224640404 | 0.017396521 | 0.305108209 | 2.165606551 |
| creatine | C00300 | 3.164706233 | 0.014597081 | 0.277344531 | 1.33537068 |
| cytidine | C00475 | 3.00590461 | 0.027194561 | 0.401769646 | 2.333657123 |
| lactate | C00186 | 2.953716944 | 0.013197361 | 0.277344531 | 1.388641177 |
| cytidine_5′-monophosphate | C00055 | 2.879610664 | 0.013797241 | 0.277344531 | 1.568545877 |
| UDP-N-acetylglucosamine | C00043 | 2.860988679 | 0.020195961 | 0.328905647 | 1.984143569 |
| inosine | C00294 | 2.760442558 | 0.014397121 | 0.277344531 | 1.491261092 |
| histamine | C00388 | 2.536010991 | 0.048590282 | 0.443143371 | 2.471158482 |
| phenol_sulfate | C02180 | 2.4373911 | 0.054189162 | 0.457597369 | 2.039715077 |
| glutathione,_reduced | C00051 | 2.396276322 | 0.047990402 | 0.443143371 | 2.100982459 |
| 1,5-anhydroglucitol | C07326 | 2.341062169 | 0.047590482 | 0.443143371 | 1.635022329 |
| pyruvate | C00022 | 2.305345621 | 0.069386123 | 0.465295176 | 1.743049791 |
| maltotriose | C01835 | 2.290135808 | 0.080583883 | 0.483503299 | 3.655638074 |
| urea | C00086 | 2.284307214 | 0.066386723 | 0.465295176 | 2.103980913 |
| glucose-6-phosphate | C00668 | 2.279352365 | 0.064187163 | 0.465295176 | 2.329128567 |
| S-adenosylhomocysteine | C00021 | 2.273586198 | 0.032793441 | 0.439817919 | 1.352588589 |
| taurine | C00245 | 2.190941908 | 0.075784843 | 0.47685598 | 1.77187529 |
| glutathione,_oxidized | C00127 | 2.187730524 | 0.067986403 | 0.465295176 | 2.01563179 |
| maltotetraose | C02052 | 2.163987577 | 0.114177165 | 0.542341532 | 2.146561165 |
| adenosine_5′diphosphoribose | C00301 | 2.151206354 | 0.091381724 | 0.514525666 | 1.995777382 |
| 5-methylthioadenosine | C00170 | 2.102798431 | 0.056988602 | 0.464050047 | 1.341762849 |
| ascorbate | C00072 | 2.089903443 | 0.093981204 | 0.514525666 | 1.847117019 |
| mannose-6-phosphate | C00275 | 2.038634098 | 0.134373125 | 0.567353196 | 1.841302621 |
| maltose | C00208 | 1.978487143 | 0.086782643 | 0.507344685 | 2.10652292 |
| guanosine | C00387 | 1.946126345 | 0.066186763 | 0.465295176 | 1.184773035 |
| N-acetylneuraminate | C00270 | 1.874857437 | 0.212557489 | 0.660763847 | 1.684556067 |
| glutamine | C00064 | 1.864128402 | 0.111377724 | 0.542341532 | 1.283122061 |
| mannitol | C00392 | 1.85417422 | 0.159168166 | 0.613957209 | 1.771333318 |
| dehydroisoandrosterone_sulfate | C04555 | 1.853918677 | 0.123775245 | 0.547090582 | 1.411160733 |
| catechol_sulfate | C00090 | 1.800513285 | 0.124775045 | 0.547090582 | 1.588529525 |
| trans-4-hydroxyproline | C01157 | 1.795999807 | 0.161567686 | 0.613957209 | 1.442095143 |
| phenylacetylglutamine | C05597 | 1.775341794 | 0.279144171 | 0.684353452 | 2.577157033 |
| N-acetyl-aspartyl-glutamate | C12270 | 1.768713793 | 0.173365327 | 0.630226896 | 1.637257633 |
| creatinine | C00791 | 1.74789424 | 0.120975805 | 0.547090582 | 1.274099083 |
| nicotinamide | C00153 | 1.700772921 | 0.152569486 | 0.610277944 | 1.25418923 |
| N-acetylaspartate | C01042 | 1.696249859 | 0.199960008 | 0.651298312 | 1.569771486 |
| ergothioneine | C05570 | 1.646630524 | 0.185562887 | 0.630226896 | 1.307208836 |
| beta-alanine | C00099 | 1.626964981 | 0.176364727 | 0.630226896 | 1.477852965 |
| mannose | C00159 | 1.626534076 | 0.203759248 | 0.654325473 | 1.416041172 |
| tryptophan_betaine | C09213 | 1.603211561 | 0.181163767 | 0.630226896 | 1.497837098 |
| choline_phosphate | C00588 | 1.599288114 | 0.217356529 | 0.660763847 | 2.134075496 |
| piperine | C03882 | 1.587917194 | 0.208958208 | 0.660763847 | 1.496167125 |
| theobromine | C07480 | 1.542597536 | 0.25634873 | 0.671810465 | 1.699841541 |
| hippurate | C01586 | 1.532123814 | 0.23815237 | 0.66628602 | 1.87941845 |
| inositol_1-phosphate | C01177 | 1.500814292 | 0.198760248 | 0.651298312 | 1.283656967 |
| 3-methylhistidine | C01152 | 1.495733462 | 0.182563487 | 0.630226896 | 1.153833753 |
| coenzyme_A | C00010 | 1.483056194 | 0.273945211 | 0.684353452 | 1.362349373 |
| cysteinylglycine | C01419 | 1.477806304 | 0.187962408 | 0.630226896 | 1.313383292 |
| glycerol_3-phosphate | C00093 | 1.454030776 | 0.229154169 | 0.665396035 | 1.303381796 |
| adenosine_5′-diphosphate | C00008 | 1.431174873 | 0.236352729 | 0.66628602 | 1.484766413 |
| deoxycholate | C04483 | 1.398727925 | 0.5054989 | 0.76214757 | 1.357954808 |
| phenylacetylglycine | C05598 | 1.395250142 | 0.466706659 | 0.749359987 | 1.391377916 |
| N-acetylputrescine | C02714 | 1.39274986 | 0.293341332 | 0.689503336 | 1.789939705 |
| hexanoylcarnitine | C01585 | 1.363520304 | 0.340331934 | 0.718056389 | 1.503644559 |
| 4-acetamidophenol | C06804.2 | 1.355041212 | 0.444111178 | 0.729782101 | 1.479625675 |
| nicotinamide_adenine_dinucleotide | C00003 | 1.3448852 | 0.273945211 | 0.684353452 | 1.792508776 |
| myo-inositol | C00137 | 1.333683666 | 0.24255149 | 0.66628602 | 1.28150763 |
| cholesterol | C00187 | 1.330887533 | 0.276944611 | 0.684353452 | 1.138722283 |
| 3-aminoisobutyrate | C05145 | 1.307978379 | 0.381923615 | 0.718056389 | 1.602561188 |
| adenosine | C00212 | 1.253618674 | 0.269346131 | 0.684353452 | 1.713276852 |
| phosphate | C00009 | 1.229934813 | 0.24075185 | 0.66628602 | 1.09104206 |
| penicillin_G | C05551 | 1.205383457 | 0.703059388 | 0.914378922 | 1.406842867 |
| aspartate | C00049 | 1.201319034 | 0.286942611 | 0.686237752 | 1.308740642 |
| scyllo-inositol | C06153 | 1.190527917 | 0.337732454 | 0.718056389 | 1.50662793 |
| urate | C00366 | 1.177640063 | 0.332133573 | 0.718056389 | 1.301153419 |
| 7-alpha-hydroxy-3-oxo-4-cholestenoate | C17337 | 1.176647545 | 0.343731254 | 0.718056389 | 1.281875544 |
| pipecolate | C00408 | 1.173663806 | 0.416116777 | 0.729782101 | 1.504667056 |
| nicotinamide_adenine_dinucleotide_reduced | C00004 | 1.172302867 | 0.474305139 | 0.750520241 | 1.563657688 |
| anserine | C01262 | 1.158406973 | 0.390521896 | 0.718056389 | 1.210618102 |
| paraxanthine | C13747 | 1.154235688 | 0.48930214 | 0.750872644 | 1.531871859 |
| phosphoethanolamine | C00346 | 1.142617764 | 0.348930214 | 0.718056389 | 1.494782606 |
| citrate | C00158 | 1.098733522 | 0.331533693 | 0.718056389 | 1.24868118 |
| alpha-tocopherol | C02477 | 1.085210151 | 0.387522496 | 0.718056389 | 1.290527511 |
| p-cresol_sulfate | C01468 | 1.067245671 | 0.449510098 | 0.732059302 | 1.460053194 |
| arabitol | C00532 | 1.048687356 | 0.367926415 | 0.718056389 | 1.203265501 |
| uridine_5′-diphosphate | C00015 | 1.011588945 | 0.375124975 | 0.718056389 | 1.103932441 |
| 3′-dephosphocoenzyme_A | C00882 | 1.011588945 | 0.375124975 | 0.718056389 | 1.103932441 |
| quinolinate | C03722 | 1.011588945 | 0.375124975 | 0.718056389 | 1.103932441 |
| 2′-deoxyinosine | C05512 | 1.011588945 | 0.375124975 | 0.718056389 | 1.103932441 |
| sebacate | C08277 | 1.011588945 | 0.375124975 | 0.718056389 | 1.103932441 |
| azelate | C08261 | 1.011588945 | 0.375124975 | 0.718056389 | 1.103932441 |
| 6-phosphogluconate | C00345 | 1.011588945 | 0.375124975 | 0.718056389 | 1.103932441 |
| fructose | C00095 | 0.998732523 | 0.477304539 | 0.750520241 | 1.348612629 |
| homocarnosine | C00884 | 0.973996509 | 0.433713257 | 0.729782101 | 1.118525395 |
| erythritol | C00503 | 0.968081213 | 0.366326735 | 0.718056389 | 1.223530988 |
| 2-hydroxyglutarate | C02630 | 0.903670379 | 0.49070186 | 0.750872644 | 1.239519184 |
| flavin_adenine_dinucleotide | C00016 | 0.897286084 | 0.407718456 | 0.729782101 | 1.091839845 |
| 3-phosphoglycerate | C00597 | 0.892637896 | 0.427114577 | 0.729782101 | 1.335202731 |
| glycerophosphorylcholine | C00670 | 0.892261738 | 0.415916817 | 0.729782101 | 1.256250614 |
| ribose | C00121 | 0.879737026 | 0.644071186 | 0.86892444 | 1.307059676 |
| acetylcholine | C01996 | 0.879122109 | 0.444911018 | 0.729782101 | 1.370712507 |
| xylulose | C00310 | 0.837886371 | 0.48930214 | 0.750872644 | 1.514763173 |
| 1,7-dimethylurate | C16356 | 0.836286685 | 0.464707059 | 0.749359987 | 1.091151065 |
| spermine | C00750 | 0.815371256 | 0.476704659 | 0.750520241 | 1.353323086 |
| carnosine | C00386 | 0.789437851 | 0.789842032 | 0.920337145 | 1.25247823 |
| pseudouridine | C02067 | 0.771980805 | 0.481103779 | 0.750872644 | 1.144829751 |
| xylitol | C00379 | 0.746479833 | 0.49470106 | 0.751945611 | 1.193974941 |
| agmatine | C00179 | 0.724132598 | 0.74005199 | 0.916091782 | 1.424265546 |
| 5-hydroxyindoleacetate | C05635 | 0.704121017 | 0.75164967 | 0.916091782 | 1.316137169 |
| isocitrate | C00311 | 0.697255242 | 0.515096981 | 0.762611114 | 1.206810699 |
| 2-hydroxystearate | C03045 | 0.695011635 | 0.696860628 | 0.914378922 | 1.320713987 |
| pyridoxate | C00847 | 0.694372025 | 0.547690462 | 0.790626685 | 1.083699919 |
| 4-acetaminophen_sulfate | C06804 | 0.669644149 | 0.940211958 | 0.988971436 | 1.324502651 |
| glycerol_2-phosphate | C02979 | 0.654025364 | 0.705258948 | 0.914378922 | 1.192696288 |
| galactose | C01662 | 0.63022944 | 0.903019396 | 0.985112068 | 1.335333127 |
| 2-aminobutyrate | C02261 | 0.613266453 | 0.603079384 | 0.838427436 | 1.102669936 |
| 2-hydroxybutyrate | C05984 | 0.603030254 | 0.713857229 | 0.914378922 | 1.179598702 |
| glycylleucine | C02155 | 0.53338166 | 0.771445711 | 0.916091782 | 1.147544092 |
| cis-aconitate | C00417 | 0.515881155 | 0.637072585 | 0.864598509 | 1.086153528 |
| caffeine | C07481 | 0.463470208 | 0.973205359 | 0.995026107 | 1.266641105 |
| heme | C00032 | 0.459503759 | 0.723255349 | 0.916091782 | 1.155470112 |
| 4-vinylphenol_sulfate | C05627 | 0.450840239 | 0.700859828 | 0.914378922 | 1.045559892 |
| serotonin | C00780 | 0.411133551 | 0.922615477 | 0.988971436 | 1.257767671 |
| indolelactate | C02043 | 0.407493479 | 0.711257748 | 0.914378922 | 1.042746396 |
| uridine_5′-monophosphate | C00105 | 0.386922582 | 0.74545091 | 0.916091782 | 1.059177357 |
| ribulose | C00309 | 0.386267724 | 0.735252949 | 0.916091782 | 1.131219725 |
| adenosine_5′-triphosphate | C00002 | 0.381622434 | 0.791041792 | 0.920337145 | 1.168782463 |
| histidine | C00135 | 0.378215548 | 0.74785043 | 0.916091782 | 1.053692586 |
| N-acetylthreonine | C01118 | 0.372813536 | 0.765646871 | 0.916091782 | 1.055872423 |
| glucose | C00293 | 0.359947046 | 0.783643271 | 0.920337145 | 1.194566578 |
| 3-(4-hydroxyphenyl)lactate | C03672 | 0.35990493 | 0.856628674 | 0.962124816 | 1.047229626 |
| betaine | C00719 | 0.340011458 | 0.767646471 | 0.916091782 | 1.04759208 |
| adenine | C00147 | 0.327695462 | 0.75164967 | 0.916091782 | 1.082169065 |
| 2-aminoadipate | C00956 | 0.267758001 | 0.825434913 | 0.940995801 | 1.046817541 |
| arginine | C00062 | 0.251031631 | 0.839432114 | 0.947477831 | 1.036651855 |
| gamma-tocopherol | C02483 | 0.23180112 | 0.873425315 | 0.976181234 | 1.090608496 |
| spermidine | C00315 | 0.229310575 | 0.9910018 | 0.99540092 | 1.0838126 |
| nicotinamide_ribonucleotide | C00455 | 0.182058732 | 0.892221556 | 0.985112068 | 1.133131703 |
| lidocaine | D00358 | 0.172240862 | 0.911217756 | 0.988971436 | 1.028583696 |
| succinate | C00042 | 0.158521544 | 0.903019396 | 0.985112068 | 1.051040907 |
| sorbitol | C00794 | 0.10936012 | 0.943611278 | 0.988971436 | 1.042315556 |
| cytidine_5′-diphosphocholine | C00307 | 0.10159355 | 0.942811438 | 0.988971436 | 1.016316771 |
| methyl-alpha-glucopyranoside | C03619 | 0.09719625 | 0.965406919 | 0.991498997 | 1.048610069 |
| stearoyl_sphingomyelin | C00550 | 0.093779078 | 0.958608278 | 0.988971436 | 1.029155736 |
| putrescine | C00134 | 0.092245224 | 0.98840232 | 0.99540092 | 1.059834522 |
| 2-hydroxyhippurate | C07588 | 0.06330477 | 0.99040192 | 0.99540092 | 1.00706226 |
| docosatrienoate | C16534 | 0.032201351 | 0.995001 | 0.99540092 | 1.017231183 |
| kynurenine | C00328 | 0.022919927 | 0.957808438 | 0.988971436 | 1.004661722 |
| N-acetylglucosamine | C00140 | 0.01591034 | 0.957608478 | 0.988971436 | 1.004489199 |
| stearate | C01530 | −0.01550318 | 0.99540092 | 0.99540092 | 1.002009807 |
| N-acetylmethionine | C02712 | −0.020115272 | 0.941611678 | 0.988971436 | 1.003991726 |
| guanine | C00242 | −0.035556161 | 0.921415717 | 0.988971436 | 1.01052316 |
| sphingosine | C00319 | −0.037883518 | 0.949810038 | 0.988971436 | 1.023790417 |
| quinate | C00296 | −0.098178665 | 0.894021196 | 0.985112068 | 1.050596138 |
| deoxycarnitine | C01181 | −0.114532588 | 0.902219556 | 0.985112068 | 1.015591531 |
| proline | C00148 | −0.197447279 | 0.830633873 | 0.942211558 | 1.021668257 |
| alanine | C00041 | −0.222379819 | 0.799240152 | 0.920337145 | 1.025560863 |
| cysteine | C00097 | −0.23066041 | 0.795240952 | 0.920337145 | 1.071373533 |
| gluconate | C00257 | −0.240452951 | 0.948810238 | 0.988971436 | 1.204717508 |
| choline | C00114 | −0.243434682 | 0.796640672 | 0.920337145 | 1.021974646 |
| acetylcarnitine | C02571 | −0.244371553 | 0.807238552 | 0.924876331 | 1.049967419 |
| 1-linoleoylglycerophosphocholine | C04100 | −0.254889749 | 0.75664867 | 0.916091782 | 1.135681491 |
| propionylcarnitine | C03017 | −0.275132712 | 0.74245151 | 0.916091782 | 1.061850266 |
| saccharopine | C00449 | −0.28725988 | 0.76044791 | 0.916091782 | 1.046367894 |
| palmitate | C00249 | −0.354014159 | 0.712457508 | 0.914378922 | 1.039708784 |
| adenosine_5′-monophosphate | C00020 | −0.365312674 | 0.680663867 | 0.91288409 | 1.102051391 |
| alpha-ketoglutarate | C00026 | −0.365833807 | 0.768846231 | 0.916091782 | 1.276380806 |
| N-acetylalanine | C02847 | −0.375076775 | 0.684663067 | 0.91288409 | 1.058891644 |
| glycerophosphoethanolamine | C01233 | −0.459162522 | 0.616676665 | 0.847001684 | 1.370587487 |
| valine | C00183 | −0.485648159 | 0.607478504 | 0.839424842 | 1.061104128 |
| malate | C00149 | −0.506537595 | 0.599880024 | 0.838427436 | 1.087512039 |
| hypoxanthine | C00262 | −0.511442281 | 0.623675265 | 0.851484793 | 1.072294605 |
| N-ethylglycinexylidide | C16561 | −0.517259887 | 0.563487303 | 0.803362309 | 1.679342042 |
| gamma-aminobutyrate | C00334 | −0.517932626 | 0.567286543 | 0.803362309 | 1.217157809 |
| xanthine | C00385 | −0.518175386 | 0.585082983 | 0.823450125 | 1.307873952 |
| 4-hydroxybutyrate | C00989 | −0.558487839 | 0.542491502 | 0.790626685 | 1.333623809 |
| carnitine | C00487 | −0.573912686 | 0.565886823 | 0.803362309 | 1.053788746 |
| myristate | C06424 | −0.580215656 | 0.547890422 | 0.790626685 | 1.057091142 |
| 1-palmitoylglycerophosphocholine | C04102 | −0.582903686 | 0.535692861 | 0.787986919 | 1.227319819 |
| fumarate | C00122 | −0.590787109 | 0.51169766 | 0.762529847 | 1.201943393 |
| pantothenate | C00864 | −0.61947029 | 0.50809838 | 0.76214757 | 1.122139149 |
| hypotaurine | C00519 | −0.686134721 | 0.438912218 | 0.729782101 | 1.404934212 |
| citrulline | C00327 | −0.71721526 | 0.439712058 | 0.729782101 | 1.154093441 |
| N6-acetyllysine | C02727 | −0.726622361 | 0.422915417 | 0.729782101 | 1.158806251 |
| nicotinamide_riboside | C03150 | −0.735671863 | 0.432313537 | 0.729782101 | 1.333837262 |
| ethanolamine | C00189 | −0.74410792 | 0.426714657 | 0.729782101 | 1.147102652 |
| serine | C00065 | −0.756829716 | 0.416316737 | 0.729782101 | 1.139770884 |
| threonine | C00188 | −0.769546045 | 0.412317536 | 0.729782101 | 1.13742408 |
| fucose | C00382 | −0.781190415 | 0.378924215 | 0.718056389 | 1.304574983 |
| glycine | C00037 | −0.800035095 | 0.431513697 | 0.729782101 | 1.099792275 |
| sarcosine | C00213 | −0.809416649 | 0.365126975 | 0.718056389 | 1.37892641 |
| N-acetyltryptophan | C03137 | −0.813114177 | 0.285742851 | 0.686237752 | 2.372048471 |
| asparagine | C00152 | −0.852730293 | 0.385122975 | 0.718056389 | 1.149741446 |
| 1-arachidonylglycerol | C13857 | −0.863941256 | 0.344131174 | 0.718056389 | 1.154020568 |
| ornithine | C00077 | −0.887208148 | 0.334733053 | 0.718056389 | 1.304123674 |
| butyrylcarnitine | C02862 | −0.907591738 | 0.351329734 | 0.718056389 | 1.230570118 |
| 5,6-dihydrouracil | C00429 | −0.932680872 | 0.337532494 | 0.718056389 | 1.358845909 |
| 1-oleoylglycerophosphocholine | C03916 | −0.974716816 | 0.25614877 | 0.671810465 | 1.746513462 |
| glycerate | C00258 | −1.011392979 | 0.288942212 | 0.686237752 | 1.250272854 |
| 1-stearoylglycerol | D01947 | −1.014411909 | 0.311537692 | 0.717480746 | 1.248927124 |
| isoleucine | C00407 | −1.044635456 | 0.266746651 | 0.684353452 | 1.127636431 |
| N1-methyladenosine | C02494 | −1.047291868 | 0.308738252 | 0.717480746 | 1.105498762 |
| 3-hydroxybutyrate | C01089 | −1.101027329 | 0.217356529 | 0.660763847 | 1.838434169 |
| 5-oxoproline | C01879 | −1.104600786 | 0.24895021 | 0.671810465 | 1.21147617 |
| tryptophan | C00078 | −1.12193614 | 0.230553889 | 0.665396035 | 1.19928843 |
| ribitol | C00474 | −1.125816518 | 0.223555289 | 0.665396035 | 1.374021712 |
| methionine | C00073 | −1.129416374 | 0.178564287 | 0.630226896 | 1.243998417 |
| nonadecanoate | C16535 | −1.151378406 | 0.225354929 | 0.665396035 | 1.456461102 |
| glutarate | C00489 | −1.196421325 | 0.142371526 | 0.586168481 | 2.141975907 |
| glutamate | C00025 | −1.254041416 | 0.25374925 | 0.671810465 | 1.105792357 |
| lysine | C00047 | −1.254680803 | 0.161567686 | 0.613957209 | 1.478345503 |
| docosapentaenoate | C16513 | −1.265656087 | 0.179364127 | 0.630226896 | 1.449894385 |
| dimethylarginine | C03626 | −1.267623363 | 0.143971206 | 0.586168481 | 1.467794919 |
| eicosapentaenoate | C06428 | −1.38501701 | 0.114177165 | 0.542341532 | 1.609937133 |
| riboflavin | C00255 | −1.419242579 | 0.109378124 | 0.542341532 | 1.384043971 |
| linoleate | C01595 | −1.435954261 | 0.119576085 | 0.547090582 | 1.354780572 |
| 3-dehydrocarnitine | C02636 | −1.534202089 | 0.100379924 | 0.532247039 | 1.431415076 |
| adrenate | C16527 | −1.549864721 | 0.113577285 | 0.542341532 | 1.361190854 |
| tyrosine | C00082 | −1.555221319 | 0.094781044 | 0.514525666 | 1.214296185 |
| glycerol | C00116 | −1.589969819 | 0.132973405 | 0.567353196 | 1.188535382 |
| palmitoleate | C08362 | −1.597100074 | 0.072185563 | 0.470237381 | 1.380817004 |
| cystine | C00491 | −1.618523501 | 0.043591282 | 0.443143371 | 2.303976537 |
| guanosine_5′-_monophosphate | C00144 | −1.653433932 | 0.077384523 | 0.47685598 | 1.460997765 |
| phenylalanine | C00079 | −1.676772892 | 0.064187163 | 0.465295176 | 1.267094175 |
| dihomo-linoleate | C16525 | −1.748264892 | 0.039392122 | 0.443143371 | 1.869440782 |
| linolenate_[alpha_or_gamma_(18:3n3_or_6)] | C06427 | −1.786221798 | 0.053989202 | 0.457597369 | 1.502230891 |
| leucine | C00123 | −1.795683069 | 0.035792841 | 0.443143371 | 1.368682405 |
| uracil | C00106 | −1.819705221 | 0.037392521 | 0.443143371 | 1.882501068 |
| docosapentaenoate | C06429.2 | −1.861003744 | 0.00239952 | 0.078155797 | 2.727908389 |
| docosadienoate | C16533 | −1.879714016 | 0.041591682 | 0.443143371 | 1.872861712 |
| docosahexaenoate | C06429 | −2.051674201 | 0.014197161 | 0.277344531 | 1.949122819 |
| arachidonate | C00219 | −2.199592552 | 0.028194361 | 0.401769646 | 1.49143101 |
| xanthosine | C01762 | −2.766594703 | 0.0019996 | 0.078155797 | 2.98512127 |
| dihomo-linolenate | C03242 | −3.016825355 | 0.00219956 | 0.078155797 | 2.115186614 |
| cis-vaccenate | C08367 | −3.242499914 | 0.00079984 | 0.078155797 | 2.464331393 |
| oleate | C00712 | −3.455677401 | 0.0019996 | 0.078155797 | 1.718089283 |

TABLE 3
List of metabolite sets tested by GSEA in RWPE-AKT1 cells, MPAKT mice and phosphoAKT1-high/MYC-low
tumors compared to RWPE-MYC cells, Lo-MYC mice and MYC-high/phosphoAKT1-low tumors, respectively.

| | | | | | | |
|---|---|---|---|---|---|---|
| | No of | Normalized | | | | RANK |
| | metab- | Enrichment | NOM | FDR | FWER | AT |
| Metabolite set | olites | Score | p-val | q-val | p-val | MAX |

Table 3: GSEA RWPE-AKT1

| | | | | | | |
|---|---|---|---|---|---|---|
| PENTOSE_PHOSPHATE_PATHWAY | 4 | 1.460002 | 0.028629856 | 0.9964033 | 0.565 | 2 |
| FRUCTOSE_AND_MANNOSE_METABOLISM | 4 | 1.4568312 | 0.12215321 | 0.50753045 | 0.573 | 1 |
| GLYCOLYSIS_GLUCONEOGENESIS | 5 | 1.3630538 | 0.15853658 | 0.7315131 | 0.792 | 13 |
| BIOSYNTHESIS_OF_UNSATURATED_FATTY_ACIDS | 9 | 1.2915634 | 0.24528302 | 0.8947219 | 0.937 | 11 |
| AMINO_SUGAR_AND_NUCLEOTIDE_SUGAR_METABOLISM | 7 | 1.2851669 | 0.21052632 | 0.7534751 | 0.944 | 7 |
| FATTY_ACID_METABOLISM | 2 | 1.2704923 | 0.14541833 | 0.682325 | 0.95 | 8 |
| PORPHYRIN_AND_CHLOROPHYLL_METABOLISM | 3 | 1.2340059 | 0.10080645 | 0.71324664 | 0.973 | 33 |
| D-GLUTAMINE_AND_D-GLUTAMATE_METABOLISM | 2 | 1.2266324 | 0.15240084 | 0.64646983 | 0.975 | 18 |
| LYSINE_DEGRADATION | 3 | 1.1647791 | 0.23246492 | 0.7812183 | 0.993 | 42 |
| VALINE_LEUCINE_AND_ISOLEUCINE_BIOSYNTHESIS | 4 | 1.1625785 | 0.24901961 | 0.7165096 | 0.997 | 36 |
| TRYPTOPHAN_METABOLISM | 2 | 1.1446722 | 0.156 | 0.7044717 | 0.999 | 4 |
| PHENYLALANINE_TYROSINE_AND_TRYPTOPHAN_BIOSYNTHESIS | 3 | 1.1393136 | 0.2672065 | 0.6605307 | 0.999 | 32 |
| SPHINGOLIPID_METABOLISM | 2 | 1.0989345 | 0.46747968 | 0.72400373 | 0.999 | 27 |
| LINOLEIC_ACID_METABOLISM | 2 | 1.0814053 | 0.4389313 | 0.72070676 | 1 | 10 |
| VALINE_LEUCINE_AND_ISOLEUCINE_DEGRADATION | 3 | 1.0684689 | 0.40944883 | 0.7040403 | 1 | 36 |
| PURINE_METABOLISM | 15 | 1.0494529 | 0.418 | 0.6976 | 1 | 18 |
| GLYOXYLATE_AND_DICARBOXYLATE_METABOLISM | 6 | 0.98523366 | 0.4792531 | 0.79075235 | 1 | 39 |
| PROPANOATE_METABOLISM | 3 | 0.97056115 | 0.54980844 | 0.7753743 | 1 | 47 |
| STARCH_AND_SUCROSE_METABOLISM | 6 | 0.96169555 | 0.43835616 | 0.748519 | 1 | 0 |
| PRIMARY_BILE_ACID_BIOSYNTHESIS | 2 | 0.8673413 | 0.7649484 | 0.87141985 | 1 | 57 |
| PENTOSE_AND_GLUCURONATE_INTERCONVERSIONS | 2 | 0.8546371 | 0.7352342 | 0.8472796 | 1 | 21 |
| GALACTOSE_METABOLISM | 6 | 0.85244286 | 0.6832298 | 0.8113915 | 1 | 0 |
| BUTIROSIN_AND_NEOMYCIN_BIOSYNTHESIS | 2 | 0.785421 | 0.7838384 | 0.86009914 | 1 | 55 |
| CYANOAMINO_ACID_METABOLISM | 5 | 0.74070346 | 0.85626286 | 0.87580043 | 1 | 28 |
| ASCORBATE_AND_ALDARATE_METABOLISM | 5 | 0.66054755 | 0.83433133 | 0.9222075 | 1 | 22 |
| D-ARGININE_AND_D-ORNITHINE_METABOLISM | 2 | 0.49286503 | 0.9831933 | 0.98765165 | 1 | 81 |

Table 3: GSEA-RWPE-MYC

| | | | | | | |
|---|---|---|---|---|---|---|
| PANTOTHENATE_AND_COA_BIOSYNTHESIS | 6 | −1.3073608 | 0.098196395 | 1 | 0.933 | 14 |
| BETA-ALANINE_METABOLISM | 8 | −1.237877 | 0.20564516 | 1 | 0.969 | 24 |
| NICOTINATE_AND_NICOTINAMIDE_METABOLISM | 5 | −1.1971127 | 0.16875 | 1 | 0.988 | 19 |
| LYSINE_BIOSYNTHESIS | 2 | −1.1673465 | 0.20272905 | 1 | 0.988 | 14 |
| GLYCEROPHOSPHOLIPID_METABOLISM | 5 | −1.1504487 | 0.312 | 1 | 0.997 | 11 |
| BUTANOATE_METABOLISM | 3 | −1.1440222 | 0.2929293 | 1 | 0.997 | 19 |
| TAURINE_AND_HYPOTAURINE_METABOLISM | 5 | −1.1243932 | 0.26899385 | 1 | 0.997 | 43 |
| INOSITOL_PHOSPHATE_METABOLISM | 3 | −1.0681249 | 0.42519686 | 1 | 1 | 37 |
| PYRUVATE_METABOLISM | 4 | −1.0595751 | 0.37475345 | 1 | 1 | 2 |
| GLYCEROLIPID_METABOLISM | 3 | −1.0349437 | 0.49501 | 1 | 1 | 31 |
| OXIDATIVE_PHOSPHORYLATION | 7 | −1.0332325 | 0.4569672 | 0.9870848 | 1 | 25 |
| ALANINE_ASPARTATE_AND_GLUTAMATE_METABOLISM | 8 | −1.0102847 | 0.47368422 | 0.9652419 | 1 | 28 |
| ARGININE_AND_PROLINE_METABOLISM | 13 | −1.006397 | 0.4853229 | 0.9018485 | 1 | 24 |
| CYSTEINE_AND_METHIONINE_METABOLISM | 8 | −0.9592696 | 0.50395256 | 0.9378031 | 1 | 35 |
| HISTIDINE_METABOLISM | 3 | −0.95365137 | 0.5246548 | 0.88732415 | 1 | 14 |
| FATTY_ACID_BIOSYNTHESIS | 7 | −0.9490036 | 0.55220884 | 0.8413623 | 1 | 29 |
| GLUTATHIONE_METABOLISM | 12 | −0.93477863 | 0.4864865 | 0.81286883 | 1 | 35 |
| CITRATE_CYCLE_TCA_CYCLE | 3 | −0.90559417 | 0.5530146 | 0.83068776 | 1 | 40 |
| PYRIMIDINE_METABOLISM | 8 | −0.8964586 | 0.562249 | 0.8050745 | 1 | 13 |
| GLYCINE_SERINE_AND_THREONINE_METABOLISM | 9 | −0.7700872 | 0.6830266 | 0.9393847 | 1 | 30 |
| TYROSINE_METABOLISM | 2 | −0.769539 | 0.73913044 | 0.895587 | 1 | 19 |
| PHENYLALANINE_METABOLISM | 3 | −0.5310729 | 0.9564356 | 1 | 1 | 19 |
| THIAMINE_METABOLISM | 3 | −0.48144296 | 0.97475725 | 1 | 1 | 30 |
| SULFUR_METABOLISM | 2 | −0.44120446 | 0.9849906 | 0.9952599 | 1 | 30 |

Table 3: GSEA MPAKT

| | | | | | | |
|---|---|---|---|---|---|---|
| PROPANOATE_METABOLISM | 3 | 1.4212209 | 0.007677543 | 1 | 0.654 | 11 |
| RIBOFLAVIN_METABOLISM | 3 | 1.372716 | 0.09445585 | 1 | 0.75 | 22 |
| PYRUVATE_METABOLISM | 2 | 1.3104335 | 0.07984791 | 1 | 0.877 | 12 |
| VALINE_LEUCINE_AND_ISOLEUCINE_DEGRADATION | 3 | 1.2896582 | 0.09981167 | 0.9193679 | 0.904 | 24 |
| GLYCOLYSIS_GLUCONEOGENESIS | 3 | 1.2842201 | 0.11821705 | 0.76036984 | 0.909 | 28 |
| FRUCTOSE_AND_MANNOSE_METABOLISM | 5 | 1.2186812 | 0.23224568 | 0.9087855 | 0.963 | 39 |
| VALINE_LEUCINE_AND_ISOLEUCINE_BIOSYNTHESIS | 4 | 1.203439 | 0.20229007 | 0.83821553 | 0.967 | 36 |
| SPHINGOLIPID_METABOLISM | 2 | 1.1720407 | 0.2967864 | 0.8420814 | 0.983 | 7 |
| CYANOAMINO_ACID_METABOLISM | 4 | 1.1263003 | 0.3490566 | 0.91562194 | 0.988 | 23 |
| CITRATE_CYCLE_TCA_CYCLE | 4 | 1.0926877 | 0.40726578 | 0.9433773 | 0.991 | 13 |
| LYSINE_BIOSYNTHESIS | 2 | 1.0827181 | 0.4215501 | 0.89252335 | 0.992 | 34 |
| LYSINE_DEGRADATION | 3 | 1.0561596 | 0.43202978 | 0.893319 | 0.994 | 34 |
| INOSITOL_PHOSPHATE_METABOLISM | 3 | 1.0481584 | 0.45901638 | 0.84673667 | 0.994 | 9 |
| PHENYLALANINE_TYROSINE_AND_TRYPTOPHAN_BIOSYNTHESIS | 3 | 1.030014 | 0.46780303 | 0.83334106 | 0.998 | 46 |
| PENTOSE_PHOSPHATE_PATHWAY | 6 | 0.99357647 | 0.541502 | 0.8660383 | 0.999 | 52 |
| GLYOXYLATE_AND_DICARBOXYLATE_METABOLISM | 9 | 0.99266577 | 0.4915254 | 0.81275177 | 0.999 | 18 |
| PRIMARY_BILE_ACID_BIOSYNTHESIS | 4 | 0.97943664 | 0.47991967 | 0.7916929 | 0.999 | 19 |
| PHENYLALANINE_METABOLISM | 4 | 0.9507707 | 0.55893534 | 0.80092 | 0.999 | 46 |
| GALACTOSE_METABOLISM | 6 | 0.9412344 | 0.6054159 | 0.77208287 | 0.999 | 33 |
| THIAMINE_METABOLISM | 4 | 0.934541 | 0.5882353 | 0.744193 | 0.999 | 18 |
| SULFUR_METABOLISM | 2 | 0.820349 | 0.72121215 | 0.8819321 | 1 | 7 |
| VITAMIN_B6_METABOLISM | 2 | 0.79861397 | 0.78313255 | 0.86963636 | 1 | 22 |
| PANTOTHENATE_AND_COA_BIOSYNTHESIS | 6 | 0.6654045 | 0.85265225 | 0.9783193 | 1 | 23 |
| AMINO_SUGAR_AND_NUCLEOTIDE_SUGAR_METABOLISM | 8 | 0.6636729 | 0.83472806 | 0.93915343 | 1 | 39 |
| STEROID_BIOSYNTHESIS | 2 | 0.6605123 | 0.85685885 | 0.9049515 | 1 | 19 |
| BETA-ALANINE_METABOLISM | 6 | 0.6585342 | 0.86159843 | 0.871574 | 1 | 26 |

Table 3: GSEA Lo-MYC

| | | | | | | |
|---|---|---|---|---|---|---|
| BIOSYNTHESIS_OF_UNSATURATED_FATTY_ACIDS | 9 | −1.4511175 | 0.05380334 | 0.99184 | 0.59 | 33 |
| LINOLEIC_ACID_METABOLISM | 3 | −1.3828204 | 0.021857923 | 0.8998 | 0.772 | 13 |
| ARGININE_AND_PROLINE_METABOLISM | 12 | −1.368322 | 0.13865547 | 0.66961 | 0.803 | 10 |
| D-GLUTAMINE_AND_D-GLUTAMATE_METABOLISM | 2 | −1.3359506 | 0.096114516 | 0.60689 | 0.848 | 10 |
| TAURINE_AND_HYPOTAURINE_METABOLISM | 5 | −1.302605 | 0.13806707 | 0.605 | 0.908 | 24 |
| PYRIMIDINE_METABOLISM | 13 | −1.2765912 | 0.16359918 | 0.58182 | 0.939 | 7 |
| PURINE_METABOLISM | 15 | −1.1867205 | 0.20042194 | 0.78346 | 0.976 | 25 |
| ASCORBATE_AND_ALDARATE_METABOLISM | 4 | −1.151681 | 0.27309236 | 0.80321 | 0.983 | 7 |
| PENTOSE_AND_GLUCURONATE_INTERCONVERSIONS | 6 | −1.126041 | 0.296 | 0.78761 | 0.992 | 7 |
| GLYCINE_SERINE_AND_THREONINE_METABOLISM | 12 | −1.0248519 | 0.428 | 1 | 0.996 | 8 |
| ARACHIDONIC_ACID_METABOLISM | 2 | −0.998357 | 0.5139442 | 0.97612 | 1 | 9 |
| GLYCEROLIPID_METABOLISM | 4 | −0.9906563 | 0.5187377 | 0.91307 | 1 | 7 |
| ALANINE_ASPARTATE_AND_GLUTAMATE_METABOLISM | 4 | −0.98792636 | 0.5254583 | 0.84914 | 1 | 10 |
| HISTIDINE_METABOLISM | 5 | −0.94616646 | 0.5529865 | 0.86822 | 1 | 10 |
| GLYCEROPHOSPHOLIPID_METABOLISM | 7 | −0.9143583 | 0.606403 | 0.86177 | 1 | 27 |
| FATTY_ACID_BIOSYNTHESIS | 4 | −0.8648357 | 0.65252525 | 0.89381 | 1 | 44 |
| STARCH_AND_SUCROSE_METABOLISM | 6 | −0.83841366 | 0.6825397 | 0.87951 | 1 | 7 |
| GLUTATHIONE_METABOLISM | 7 | −0.8241191 | 0.6639511 | 0.85319 | 1 | 24 |
| NICOTINATE_AND_NICOTINAMIDE_METABOLISM | 3 | −0.7469816 | 0.7590361 | 0.90931 | 1 | 32 |
| PORPHYRIN_AND_CHLOROPHYLL_METABOLISM | 3 | −0.7453042 | 0.79352224 | 0.86597 | 1 | 10 |
| CYSTEINE_AND_METHIONINE_METABOLISM | 9 | −0.69749016 | 0.8177966 | 0.87575 | 1 | 26 |
| UBIQUINONE_AND_OTHER_TERPENOID- | 2 | −0.60078293 | 0.9536842 | 0.9168 | 1 | 91 |
| QUINONE_BIOSYNTHESIS | | | | | | |

Table 3: GSEA PhosphoAKIT1-high tumors

| | | | | | | |
|---|---|---|---|---|---|---|
| GLYCOLYSIS_GLUCONEOGENESIS | 4 | 1.5907214 | 0 | 0.46191543 | 0.332 | 16 |
| AMINO_SUGAR_AND_NUCLEOTIDE_SUGAR_METABOLISM | 7 | 1.5328926 | 0.020072993 | 0.42452946 | 0.504 | 40 |
| PYRIMIDINE_METABOLISM | 12 | 1.4802719 | 0.052892562 | 0.45880678 | 0.674 | 39 |
| PYRUVATE_METABOLISM | 3 | 1.4683071 | 0.026 | 0.3751393 | 0.702 | 13 |
| PENTOSE_PHOSPHATE_PATHWAY | 7 | 1.4230571 | 0.095 | 0.44165498 | 0.82 | 16 |
| STARCH_AND_SUCROSE_METABOLISM | 4 | 1.3226093 | 0.10642202 | 0.7378345 | 0.961 | 25 |
| FRUCTOSE_AND_MANNOSE_METABOLISM | 6 | 1.3132623 | 0.13768116 | 0.671879 | 0.966 | 40 |
| CYSTEINE_AND_METHIONINE_METABOLISM | 11 | 1.2838272 | 0.19607843 | 0.70322126 | 0.98 | 22 |
| ASCORBATE_AND_ALDARATE_METABOLISM | 4 | 1.242083 | 0.21402878 | 0.7720861 | 0.992 | 58 |
| PROPANOATE_METABOLISM | 5 | 1.1808307 | 0.29681274 | 0.92195565 | 0.998 | 39 |
| NICOTINATE_AND_NICOTINAMIDE_METABOLISM | 8 | 1.1765169 | 0.274276 | 0.8520035 | 0.998 | 80 |
| ARGININE_AND_PROLINE_METABOLISM | 21 | 1.1571836 | 0.29952458 | 0.8452255 | 0.999 | 35 |
| INOSITOL_PHOSPHATE_METABOLISM | 3 | 1.1525284 | 0.31501058 | 0.79334915 | 0.999 | 64 |
| TAURINE_AND_HYPOTAURINE_METABOLISM | 7 | 1.1355695 | 0.34843206 | 0.78442246 | 0.999 | 18 |
| STEROID_HORMONE_BIOSYNTHESIS | 2 | 1.0969528 | 0.4081238 | 0.8339776 | 0.999 | 61 |
| BUTIROSIN_AND_NEOMYCIN_BIOSYNTHESIS | 2 | 1.0847456 | 0.38477367 | 0.8128595 | 0.999 | 7 |
| PURINE_METABOLISM | 18 | 1.0811335 | 0.38162544 | 0.77331376 | 0.999 | 65 |
| VITAMIN_B6_METABOLISM | 3 | 1.0634779 | 0.43485916 | 0.77049446 | 1 | 13 |
| HISTIDINE_METABOLISM | 9 | 1.0361688 | 0.3986135 | 0.78980684 | 1 | 69 |
| OXIDATIVE_PHOSPHORYLATION | 7 | 1.0176637 | 0.48181817 | 0.7894189 | 1 | 76 |
| PRIMARY_BILE_ACID_BIOSYNTHESIS | 4 | 0.9972558 | 0.5371094 | 0.79108274 | 1 | 66 |
| ALANINE_ASPARTATE_AND_GLUTAMATE_METABOLISM | 11 | 0.94110465 | 0.5704698 | 0.8591216 | 1 | 37 |
| GLUTATHIONE_METABOLISM | 12 | 0.92024606 | 0.5968992 | 0.8611452 | 1 | 23 |
| GLYCINE_SERINE_AND_THREONINE_METABOLISM | 12 | 0.91994816 | 0.5643739 | 0.82558614 | 1 | 13 |
| GLYCEROPHOSPHOLIPID_METABOLISM | 9 | 0.9101822 | 0.5694915 | 0.80967337 | 1 | 92 |
| TYROSINE_METABOLISM | 5 | 0.8141563 | 0.73867595 | 0.921916 | 1 | 13 |
| GALACTOSE_METABOLISM | 6 | 0.7993824 | 0.7218045 | 0.9076516 | 1 | 84 |
| D-GLUTAMINE_AND_D-GLUTAMATE_METABOLISM | 3 | 0.79120994 | 0.7649186 | 0.88606244 | 1 | 28 |
| PHENYLALANINE_METABOLISM | 7 | 0.7823577 | 0.771518 | 0.8666423 | 1 | 53 |
| PANTOTHENATE_AND_COA_BIOSYNTHESIS | 10 | 0.7694747 | 0.74523395 | 0.8529612 | 1 | 79 |
| THIAMINE_METABOLISM | 4 | 0.7329624 | 0.80626225 | 0.87004304 | 1 | 13 |
| CITRATE_CYCLE_TCA_CYCLE | 8 | 0.64468 | 0.85315984 | 0.9347561 | 1 | 13 |
| PENTOSE_AND_GLUCURONATE_INTERCONVERSIONS | 7 | 0.598778 | 0.90226877 | 0.9441087 | 1 | 98 |
| GLYOXYLATE_AND_DICARBOXYLATE_METABOLISM | 12 | 0.5758591 | 0.9124767 | 0.933276 | 1 | 28 |

Table 3: GSEA MTC-high tumors

| | | | | | | |
|---|---|---|---|---|---|---|
| BIOSYNTHESIS_OF_UNSATURATED_FATTY_ACIDS | 13 | −1.6898948 | 0.004338395 | 0.17238313 | 0.18 | 26 |
| LINOLEIC_ACID_METABOLISM | 3 | −1.405524 | 0.0480167 | 0.9980702 | 0.823 | 22 |
| PHENYLALANINE_TYROSINE_AND_TRYPTOPHAN_BIOSYNTHESIS | 4 | −1.3494385 | 0.09210526 | 0.94579667 | 0.914 | 32 |
| FATTY_ACID_BIOSYNTHESIS | 5 | −1.3365041 | 0.1594203 | 0.7610707 | 0.931 | 17 |
| PORPHYRIN_AND_CHLOROPHYLL_METABOLISM | 4 | −1.1784091 | 0.31692913 | 1 | 0.992 | 50 |
| LYSINE_DEGRADATION | 9 | −1.129812 | 0.33248731 | 1 | 0.996 | 61 |
| VALINE_LEUCINE_AND_ISOLEUCINE_DEGRADATION | 3 | −1.0934087 | 0.36734694 | 1 | 0.999 | 68 |
| RIBOFLAVIN_METABOLISM | 3 | −1.06163 | 0.44469026 | 1 | 1 | 31 |
| CYANOAMINO_ACID_METABOLISM | 5 | −0.99628294 | 0.5220264 | 1 | 1 | 51 |
| D-ARGININE_AND_D-ORNITHINE_METABOLISM | 2 | −0.9939161 | 0.49372384 | 1 | 1 | 43 |
| SULFUR_METABOLISM | 3 | −0.97494125 | 0.51096493 | 1 | 1 | 109 |
| GLYCEROLIPID_METABOLISM | 3 | −0.85183764 | 0.65784115 | 1 | 1 | 39 |
| TRYPTOPHAN_METABOLISM | 6 | −0.8230189 | 0.7038044 | 1 | 1 | 149 |
| UBIQUINONE_AND_OTHER_TERPENOID- | 4 | −0.79604733 | 0.7002342 | 1 | 1 | 19 |
| QUINONE_BIOSYNTHESIS | | | | | | |
| CAFFEINE_METABOLISM | 6 | −0.750715 | 0.71938777 | 1 | 1 | 5 |
| SPHINGOLIPID_METABOLISM | 4 | −0.6737419 | 0.89498806 | 1 | 1 | 158 |
| BUTANOATE_METABOLISM | 9 | −0.6569208 | 0.86493504 | 1 | 1 | 71 |
| VALINE_LEUCINE_AND_ISOLEUCINE_BIOSYNTHESIS | 5 | −0.6417897 | 0.8487395 | 1 | 1 | 50 |
| ETHER_LIPID_METABOLISM | 2 | −0.63222766 | 0.9 | 1 | 1 | 139 |
| LYSINE_BIOSYNTHESIS | 5 | −0.5990712 | 0.943662 | 0.9970749 | 1 | 166 |
| BETA-ALANINE_METABOLISM | 12 | −0.5383798 | 0.9814324 | 0.99758583 | 1 | 190 |
| FATTY_ACID_METABOLISM | 3 | −0.50268257 | 0.98547214 | 0.9723302 | 1 | 28 |

The foregoing written specification is considered to be sufficient to enable one skilled in the art to practice the invention. The present invention is not to be limited in scope by examples provided, since the examples are intended as a single illustration of one or more aspects of the invention and other functionally equivalent embodiments are within the scope of the invention.

Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method to identify Akt1 and Myc status in a prostate tumor comprising:
performing an assay to measure a profile of metabolites in a prostate tumor sample obtained from a subject, wherein the metabolites are differentially produced in prostate tumors with high Akt1 expression versus prostate tumors with high Myc expression; and
comparing, with at least one processor, the profile of metabolites with an appropriate reference profile of the metabolites to assign an Akt1 and Myc status to the sample based on results of the comparison.

2. A method to identify Akt1 and Myc status in a prostate tumor comprising:
analyzing, with at least one processor, a profile of a set of metabolites in a prostate tumor sample obtained from a subject to assign an Akt1 and Myc status to the sample, wherein:
the metabolites are differentially produced in prostate tumors with high Akt1 expression versus prostate tumors with high Myc expression, and
the expression profile of metabolites is compared to an appropriate reference profile of the metabolites.

3. The method of claim 1, wherein the appropriate reference profile of the metabolites comprises profiles of the metabolites in prostate tumor with high Akt1 expression, in prostate tumor with low Akt1 expression, in prostate tumor with high Myc expression, and in prostate tumor with low Myc expression.

4. The method of claim 1, wherein the metabolic profile comprises at least 5, at least 10, at least 25, at least 50, at least 75, at least 100, at least 125, at least 150, at least 175, at least 200, at least 225, at least 250, at least 275, at least 300, at least 350, at least 375, at least 400 metabolites, at least 450 metabolites, at least 500 metabolites, at least 1000 metabolites, or at least 1500 metabolites.

5. (canceled)

6. The method of claim 1, wherein the metabolites are selected from Table 1.

7. The method of claim 1, wherein the computer assigns a status of high Akt1/high Myc, high Akt1/low Myc, low Akt1/high Myc, or low Akt1/low Myc to the sample.

8-9. (canceled)

10. The method of claim 1, wherein the differentially produced metabolites are selected using a threshold of p value <0.05.

11. The method of claim 1, wherein the method further comprises:
determining a confidence value for the Akt1 and Myc status assigned to the sample; and
providing an indication of the confidence value and the Akt1 and Myc status assigned to the sample to a user.

12. A method to treat prostate tumor comprising:
obtaining a prostate tumor sample from a subject;
measuring a metabolic profile of the tumor sample, wherein the metabolites are differentially produced in prostate tumors with high Akt1 expression versus prostate tumors with high Myc expression;
comparing the metabolic profile to an appropriate reference profile of the metabolites; and
treating the subject with an Akt1 inhibitor when results of the comparison of the metabolic profile indicate high Akt1 expression in the tumor sample and/or treating the subject with a Myc inhibitor when results of the comparison of the metabolic profile indicate high Myc in the tumor sample.

13. The method of claim 12, wherein the Akt1 inhibitor is selected from the group consisting of (a) a low molecular weight compound or high molecular weight compound which inhibits the phosphorylation of Akt1, (b) a low molecular weight compound or high molecular weight compound which inhibits the expression of Akt1, (c) an antibody which inhibits the phosphorylation of Akt1, (d) an antibody which inhibits the expression of Akt1, (e) a siRNA or shRNA against a polynucleotide encoding Akt1, (f) an antisense polynucleotide comprising a nucleotide sequence complementary or substantially complementary to the nucleotide sequence of a polynucleotide encoding Akt1, or comprising a part of said nucleotide sequence, (g) a ribozyme directed to a polynucleotide encoding Akt1, (h) a mutant of Akt1 which dominant-negatively acts on Akt1 or a polynucleotide encoding said mutant, and (i) an aptamer against Akt1.

14. (canceled)

15. The method of claim 12, wherein the Myc inhibitor is selected from the group consisting of (a) a low molecular weight compound or high molecular weight compound which inhibits the expression of Myc, (b) an antibody which inhibits the expression of Myc, (e) a siRNA or shRNA against a polynucleotide encoding Myc, (f) an antisense polynucleotide comprising a nucleotide sequence complementary or substantially complementary to the nucleotide sequence of a polynucleotide encoding Myc, or comprising a part of said nucleotide sequence, (g) a ribozyme directed to a polynucleotide encoding Myc, (h) a mutant of Myc which dominant-negatively acts on Myc or a polynucleotide encoding said mutant, and (i) an aptamer against Myc.

16-17. (canceled)

18. The method of claim 12, wherein the metabolites are selected from Table 1.

19. The method of claim 12, wherein the metabolic profile of the tumor sample is compared using cluster analysis.

20. (canceled)

21. The method of claim 12, wherein the appropriate reference profile of the metabolites comprises profiles of the metabolites in prostate tumor with high Akt1 expression, in prostate tumor with low Akt1 expression, in prostate tumor with high Myc expression, and in prostate tumor with low Myc expression.

22. The method of claim 12, wherein the metabolic profile comprises at least 5, at least 10, at least 25, at least 50, at least 75, at least 100, at least 125, at least 150, at least 175, at least 200, at least 225, at least 250, at least 275, at least 300, at least 350, at least 375, at least 400 metabolites, at least 450 metabolites, at least 500 metabolites, at least 1000 metabolites, or at least 1500 metabolites.

23. (canceled)

24. A method to identify Akt1 and Myc status in a prostate tumor comprising:
performing an assay to measure a profile of metabolites in a prostate tumor sample obtained from a subject; and
comparing, with at least one processor, the profile of metabolites with a reference profile of the metabolites, the reference profile of the metabolites being profiles of the metabolites from prostate tumors with high Akt1 expression and from prostate tumors with high Myc expression, to assign an Akt1 and Myc status to the sample based on results of the comparison.

25. A method to identify Akt1 and Myc status in a prostate tumor comprising:
performing an assay to measure a profile of metabolites in a prostate tumor sample obtained from a subject; and
comparing the profile of metabolites with reference profiles of the metabolites with at least one processor programmed to recognize profiles of high Akt1 versus low Akt1 expressing tumors and high Myc versus low Myc expressing tumors; and
assigning, with at least one processor, an Akt1 and Myc status to the sample based on results of the comparison.

26. The method of claim 24, wherein the method further comprises:
determining a confidence value for the Akt1 and Myc status assigned to the sample; and
providing an indication of the confidence value and the Akt1 and Myc status assigned to the sample to a user.

27. (canceled)

28. A computer-readable storage medium encoded with a plurality of instructions that, when executed by at least one processor, performs a method comprising:
comparing the profile of metabolites with reference profiles of the metabolites with at least one processor programmed to recognize profiles of high Akt1 versus low Akt1 expressing tumors and high Myc versus low Myc expressing tumors; and
assigning, with at least one processor, an Akt1 and Myc status to the sample based on results of the comparison.

29. The computer-readable storage medium of claim 28, wherein the method further comprises:
determining a confidence value for the Akt1 and Myc status assigned to the sample; and
providing an indication of the confidence value and the Akt1 and Myc status assigned to the sample to a user.

30. (canceled)
